SEQUENCE LISTING (215 sequences)

SEQ ID NO: 1 - DNA, 333 bp, synthetic construct
  source 1..333: mol_type = other DNA
tgataataggctggagcctcggtggcctagcttcttgccccttgggcctccccccagcccctcctccccttcctgcacccgtacccccgtggtctttgaataaagtctgagtgggcggccaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 2 - DNA, 333 bp, synthetic construct
  source 1..333: mol_type = other DNA
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggaggctggagcctcggtggcctagcttcttgccccttgggcctccccccagcccctcctccccttcctgcacccgtacccccgtggtctttgaataaagtctgagtgggcggc

SEQ ID NO: 3 - DNA, 223 bp, synthetic construct
  source 1..223: mol_type = other DNA
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 4 - DNA, 119 bp, synthetic construct
  source 1..119: mol_type = other DNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - DNA, 333 bp, synthetic construct; identical to SEQ ID NO: 1
  source 1..333: mol_type = other DNA

SEQ ID NO: 6 - DNA, 57 bp, synthetic construct
  source 1..57: mol_type = other DNA
gggaaataagagagaaaagaagagtaagaagaaatataagaccccggcgccgccacc

SEQ ID NO: 7 - RNA, 121 nt, synthetic construct
  source 1..121: mol_type = other RNA
ggatccgtgatcggaaacgtgagatccacctcagatccgctaggacacccgcagatcgagaagaaggcgaattaagagagaaaagaagagtaagaagaaatataagacaccggtcgccacc

SEQ ID NO: 8 - RNA, 236 nt, synthetic construct
  source 1..236: mol_type = other RNA
gggaaacggcaagcagcagctgccgctgttttgtcttaaccggttgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggagccccggcgccgccacc

SEQ ID NO: 9 - RNA, 73 nt, synthetic construct
  source 1..73: mol_type = other RNA
gggaaataagagagaaaaagagagagggagaggagaagagtaagaagaaatataagaccccggcgccgccacc

SEQ ID NO: 10 - RNA, 73 nt, synthetic construct
  source 1..73: mol_type = other RNA
gggaaataagagagaaaagaagagtaagaaagagagagggagaggagaaatataagaccccggcgccgccacc

SEQ ID NO: 11 - RNA, 73 nt, synthetic construct
  source 1..73: mol_type = other RNA
gggaaataagagagaaaagaagagtaagaagaaatataagacagagagagggagaggacccggcgccgccacc

SEQ ID NO: 12 - RNA, 105 nt, synthetic construct
  source 1..105: mol_type = other RNA
gggaaatttttttttgatattataagagtttttttttgatattaagaaaatttttttttgatattagaagagtaagaagaaatataagaccccggcgccgccacc

SEQ ID NO: 13 - RNA, 105 nt, synthetic construct
  source 1..105: mol_type = other RNA
gggaaataagagtttttttttgatattaagaaaatttttttttgatattagaagagtttttttttgatattataagaagaaatataagaccccggcgccgccacc

SEQ ID NO: 14 - RNA, 105 nt, synthetic construct
  source 1..105: mol_type = other RNA
gggaaataagagagaaaatttttttttgatattagaagagtttttttttgatattataagaatttttttttgatattagaaatataagaccccggcgccgccacc

SEQ ID NO: 15 - RNA, 105 nt, synthetic construct
  source 1..105: mol_type = other RNA
gggaaataagagagaaaagaagagtttttttttgatattataagaatttttttttgatattagaaatatttttttttgatattataagaccccggcgccgccacc

SEQ ID NO: 16 - RNA, 105 nt, synthetic construct
  source 1..105: mol_type = other RNA
gggaaataagagagaaaagaagagtaagaatttttttttgatattagaaatatttttttttgatattataagactttttttttgatattacccggcgccgccacc

SEQ ID NO: 17 - RNA, 105 nt, synthetic construct
  source 1..105: mol_type = other RNA
gggaaataagagagaaaagaagagtaagaagaaatatttttttttgatattataagactttttttttgatattacccggctttttttttgatattagccgccacc

SEQ ID NO: 18 - RNA, 19 nt, synthetic construct
  source 1..19: mol_type = other RNA
gggccctgaagaagggccc

SEQ ID NO: 19 - RNA, 21 nt, synthetic construct
  source 1..21: mol_type = other RNA
aatccattgcactccggattt

SEQ ID NO: 20 - DNA, 244 bp, synthetic construct
  source 1..244: mol_type = other DNA
ggatgtgtggccagttggtgctgtctgtcttcttggtgaccaggagtaatttggaccatctgggaaaccagacctcttttcacccaggctgtttcgtgtgcatcacttctgaggctgtgtgctcagtcaagaccttgccagagagttgggggattagccttaggtcgagaatattctgagcagtagtaagaaatttaaaaatcatccataatttcatcactcttattttaagagattagtttat

SEQ ID NO: 21 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 22 - DNA, 351 bp, synthetic construct
  source 1..351: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctac

SEQ ID NO: 23-24 - skipped (2 entries)

SEQ ID NO: 25 - protein, 1099 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 26 - DNA, 3297 bp, synthetic construct
  source 1..3297: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 27 - protein, 1720 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSNKAPQSTGPPPAPSPGLPQPAFPPGQTAPVVFSTPQATQMNTPSQPRQHFYPSRAQPPSSAASRVQSAAPARPGPAAHVYPAGSQVMMIPSQISYPASQGAYYIPGQGRSTYVVPTQQYPVQPGAPGFYPGASPTEFGTYAGAYYPAQGVQQFPTGVAPTPVLMNQPPQIAPKRERKTIRIRDPNQGGKDITEEIMSGARTASTPTPPQTGGGLEPQANGETPQVAVIVRPDDRSQGAIIADRPGLPGPEHSPSESQPSSPSPTPSPSPVLEPGSEPNLAVLSIPGDTMTTIQMSVEESTPISRETGEPYRLSPEPTPLAEPILEVEVTLSKPVPESEFSSSPLQAPTPLASHTVEIHEPNGMVPSEDLEPEVESSPELAPPPACPSESPVPIAPTAQPEELLNGAPSPPAVDLSPVSEPEEQAKEVTASMAPPTIPSATPATAPSATSPAQEEEMEEEEEEEEGEAGEAGEAESEKGGEELLPPESTPIPANLSQNLEAAAATQVAVSVPKRRRKIKELNKKEAVGDLLDAFKEANPAVPEVENQPPAGSNPGPESEGSGVPPRPEEADETWDSKEDKIHNAENIQPGEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 28 - DNA, 5160 bp, synthetic construct
  source 1..5160: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaacaaggcccctcagagcaccggccctcctcctgcccctagccctggcctgcctcagcctgccttccctcctggccagaccgcccctgtggtgttcagcacccctcaggccacccagatgaacacacctagccagcctagacagcacttctaccctagcagagcccagcctcctagcagcgccgccagcagagtgcagtccgctgcacctgccagacctggccctgccgcccacgtgtaccctgccggcagccaggtgatgatgatcccgagtcaaatcagctaccctgcaagccagggcgcctactacatcccgggccagggcagaagcacctacgtggtgcctacccagcagtaccctgtgcagcctggcgcccctggtttctatcctggcgcaagccctaccgagttcggaacttacgccggcgcttactatccagctcagggcgtgcagcagttccctaccggcgtggcccctacccctgtgctgatgaatcagccacctcagatcgcccctaagcgcgagcgcaagaccatcagaatccgcgatcctaaccagggcggcaaggacatcaccgaggagatcatgagcggcgccagaacagcaagtactccaaccccgccacaaaccggcggcggcctggagcctcaagccaacggcgagacgccacaagtggccgtgatcgtacgccctgacgaccggagccaaggtgcaatcatcgccgataggcctggcctcccaggtccggagcacagccctagcgagtcccagccgtcttcaccatcaccaacccctagtccatcccctgttctcgaaccaggcagcgagcctaacctggccgtgctgagcataccaggtgacaccatgaccaccatccagatgagcgtggaggagagcaccccaatcagcagagaaactggagagccttacagactgtccccagagccgaccccactggccgagccaatactggaggtggaggtgaccctgagcaagcctgtgcctgagagcgagttcagctcctctccactgcaggccccaactcctctcgcaagccacaccgtggagatccacgaacctaacggcatggtaccaagcgaagatcttgagccagaggtcgaatcaagcccagaactggcccctccacctgcctgcccgtctgaatctccggtccctatcgctcctacggcacagcctgaggagctgctgaacggtgccccgagccctccagcagtggacttatccccagtatcagagcctgaagaacaggccaaggaggtaactgcctctatggcgccacctaccataccttcggcaacaccggctacagcaccatctgcgactagtccggctcaggaggaggagatggaggaagaagaagaggaagaggagggcgaggccggagaggccggtgaagccgagtccgagaagggcggcgaagaacttctccctccagagtcaactcctatccctgccaaccttagtcagaatctggaggccgccgccgctactcaggttgcagtgagcgtgccaaagagacgtcgcaagatcaaggagctgaacaagaaggaggccgtgggcgacctgctggacgccttcaaggaggcaaacccggcggtgcctgaagtggagaatcagcctccggccggatcaaaccctggtcctgagagtgaaggcagcggcgtcccaccaagacctgaagaggctgacgaaacttgggacagcaaggaggacaagatccacaacgccgagaacatccagccaggcgagcagaagtacgagtacaagagcgaccagtggaagcctcttaaccttgaagagaagaagagatacgacagagagttcctgctgggcttccagttcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 29 - protein, 1099 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 30 - DNA, 3297 bp, synthetic construct
  source 1..3297: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgcgaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 31 - protein, 1069 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 32 - DNA, 3207 bp, synthetic construct
  source 1..3207: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 33 - protein, 921 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 34 - DNA, 2763 bp, synthetic construct
  source 1..2763: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 35 - protein, 528 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSID

SEQ ID NO: 36 - DNA, 1584 bp, synthetic construct
  source 1..1584: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgac

SEQ ID NO: 37 - protein, 600 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 38 - DNA, 1800 bp, synthetic construct
  source 1..1800: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 39 - protein, 541 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 40 - DNA, 1623 bp, synthetic construct
  source 1..1623: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 41 - protein, 642 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 42 - DNA, 1926 bp, synthetic construct
  source 1..1926: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 43 - protein, 494 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 44 - DNA, 1482 bp, synthetic construct
  source 1..1482: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 45 - protein, 719 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSENSSAASASSEAGSSRSQEIEELERFIDSYVLEYQVQGLLADKTEGDGESERTQSHISQWTADCSEPLDSSCSFSRGRAPPQQNGSKDNSLDMLGTDIWAANTFDSFSGATWDLQPEKLDFTQFHRKVRHTPKQPLPHIDREGCGKGKLEDGDGINLNDIEKVLPAWQGYHPMPHEVEIAHTKKLFRRRRNDRRRQQRPPGGNKPQQHGDHQPGSAKHNRDHQKSYQGGSAPHPSGRPTHHGYSQNRRWHHGNMKHPPGDKGEAGAHRNAKETMTIENPKLEDTAGDTGHSSLEAPRSPDTLAPVASERLPPQQSGGPEVETKRKDSILPERIGERPKITLLQSSKDRLRRRLKEKDEVAVETTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 46 - DNA, 2157 bp, synthetic construct
  source 1..2157: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgagaacagtagcgccgctagcgcgagcagcgaagccgggagcagccggtctcaggagatcgaggagctggagcggttcatcgacagctacgtgctggagtaccaggtgcagggcctgctggccgacaagaccgagggcgacggcgagagcgagcggacccagagccacatcagccagtggaccgccgactgcagcgagcccctggactctagctgctcattcagtcgtggacgggcccctccacagcagaacggcagcaaggacaacagcctggacatgctgggcaccgacatctgggccgccaacaccttcgacagcttcagcggcgccacctgggatctgcagcccgagaagctggactttacccagttccaccggaaggtgcggcacactcccaagcagcccctgccccacatcgatcgggagggctgcggcaagggcaagctggaagacggcgacggcatcaacctgaacgacatcgagaaggtgctgcctgcctggcagggctaccaccccatgccccacgaggtggagatcgcccacaccaagaagctgttccggcgacgacgcaacgaccggcgtaggcagcaacggccgcctggagggaacaagccccagcagcacggagaccaccagcccggtagcgccaagcacaaccgggaccaccagaagagctaccagggcggaagcgcaccacacccctcgggcagacccacccaccacggctacagccagaaccggcggtggcatcacggtaacatgaagcacccacccggcgacaaaggagaggccggcgctcaccgtaacgccaaggagaccatgaccatcgagaaccccaagctggaggataccgccggcgatacgggtcacagcagcctggaggcaccgcggtctcccgacaccctggcacccgtggccagcgaacggctgccaccccaacagagcggcggccctgaggttgagaccaagcggaaggacagcatcctgcccgaacggatcggtgagcggcccaagatcaccttactgcagagtagcaaggaccggctgagacggcggctgaaggagaaggacgaggtggccgtggagacaaccactccccagcagaacaagatggacaagctgatcgagatcctgaacagcatgcggaacaacagcagcgacgtggacaccaagctgaccaccttcatggaggaggcccagaacagcaccaacagcgaggagatgctgggcgagatcgtgcggaccatctaccagaaggccgtgagcgaccggagcttcgccttcaccgccgccaagctgtgcgacaagatggccctgttcatggtggagggcaccaagttccggagcttactgctgaatatgctgcagaaggacttcaccgtgcgggaggagctgcagcagcaggacgtggagcggtggctgggcttcatcaccttcctgtgcgaggtgttcggcaccatgcggagcagcaccggcgaacccttccgggtgctggtgtgccccatctacacctgcctgcgggagttgctgcagagccaggacgtgaaggaggacgccgtgctgtgctgcagcatggaactgcagagcactggccggctgctggaggagcagctgcccgagatgatgaccgagctgctcgctagcgcccgggacaagatgctgtgccccagcgagagcatgctgacccggagcctgcttctggaggtgatcgagctgcacgccaacagctggaatcccctgacccctcccatcacccagtactacaaccggaccatccagaagctgaccgcc

SEQ ID NO: 47 - protein, 323 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSLNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWN

SEQ ID NO: 48 - DNA, 969 bp, synthetic construct
  source 1..969: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagcgatggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgactagatccctactgttggaggtgatcgagctgcacgccaacagctggaac

SEQ ID NO: 49 - protein, 356 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 50 - DNA, 1068 bp, synthetic construct
  source 1..1068: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaccacccctcagcagaacaagatggacaagctgatcgagatcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagaggtggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgacgcgcagcctcctgctggaggtgatcgagctgcacgccaacagctggaaccctctgacccctcctatcacccagtactacaacagaaccatccagaagctgaccgcc

SEQ ID NO: 51 - protein, 364 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPR

SEQ ID NO: 52 - DNA, 1092 bp, synthetic construct
  source 1..1092: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgccgacggcagcaagacccaggacctgttcagaagagtgagaagcatcctgaacaagctgacccctcagatgttccagcagctgatgaagcaggtgacccagctggccatcgacaccgaggagagactgaagggcgtgatcgacctgatcttcgagaaggccatcagcgagcctaacttcagcgtggcctacgccaacatgtgccggtgcctgatggccctgaaggtgcctaccaccgagaagcctaccgtgaccgtgaacttcagaaagctgctgctgaaccggtgccagaaggagttcgagaaggacaaggacgacgacgaggtgttcgagaagaagcagaaggagatggacgaggccgccaccgccgaggagagaggcagactgaaggaggagctggaggaggccagagacatcgccagaagaagaagcctgggcaacatcaagttcatcggcgagctgttcaagctgaagatgctgaccgaggccatcatgcacgactgcgtggtgaagctgctgaagaaccacgacgaggagagcctggagtgcctgtgcagactgctgaccaccatcggcaaggacctggatttcgagaaggcgaagcctagaatggaccagtacttcaaccagatggagaagatcatcaaggagaagaagaccagcagcagaatcagattcatgctgcaggacgtgctggacctgagaggcagcaactgggtgcctaga

SEQ ID NO: 53 - protein, 1168 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 54 - DNA, 3504 bp, synthetic construct
  source 1..3504: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagctcgttcacgctaacgaacaagaacgtcatcttcgtagcgggacttggcggtatcggcctagacacgtcgaaggaactactaaagcgtgacccggtagtcctccaacgtcgcgattgggagaacccgggcgtaacgcaactaaaccgtcttgcggcgcacccgccgtttgcgtcgtggcgtaactcggaggaggcgcgaacggatcgtccgtcgcaacaactacgttcgctcaacggggagtggcgcttcgcgtggttcccggcgccggaggcggtaccggagtcgtggctcgagtgcgatctaccggaggcggacacggtcgtcgtaccgtcgaactggcaaatgcacggttacgacgcgccgatatacacgaacgtcacgtacccgataacggtaaacccgccgttcgtcccgacggagaacccgacggggtgctactcgctaacgttcaacgttgacgagtcgtggttgcaagagggtcaaacgcgtatcatattcgacggtgtaaactcggcgttccacctgtggtgcaacgggcgctgggtagggtacggccaagactcgcgtctaccgtcggagttcgacctatcggcgttcctacgagcgggtgagaaccggctagcggtcatggtcctacgttggtcggacggttcgtacctcgaggaccaagacatgtggcgaatgtcgggtatcttccgcgacgtatcgctcctacacaagccgacgacgcaaatctcggacttccacgtcgcgacgcgtttcaacgacgatttctcgcgggcagtcctagaggcggaggtccaaatgtgcggggagctacgtgactacctccgtgtcacggtatcgctctggcaaggtgagacgcaagtagcgtcgggtacggcgccgttcggcggtgagatcatcgacgagcgtggtgggtacgcggaccgtgtaacgctacgtctaaacgtcgagaacccgaagctctggtcggcggagatcccgaacctataccgtgcggtcgtcgagctacatacggcggacgggacgctaatagaggcggaagcgtgcgacgtcgggtttcgagaggttcgtatagagaacgggctgctacttctaaacgggaagccgttgctcatacgtggtgtcaaccgtcacgagcaccacccgctacacggtcaagtaatggacgagcaaacgatggtacaagacatcctactaatgaagcagaacaacttcaacgcggtacgctgttcgcattacccgaaccatccgttgtggtacacgctttgcgaccgatacggtctatacgtcgtagacgaggcgaacatagagacgcacgggatggtaccgatgaatcgcctaacggacgacccgcgttggctaccggcgatgtcggagcgagtcacgcgtatggtccaacgggaccgtaaccacccgtcggtaataatctggtcgctaggcaacgaatcggggcacggggcgaaccacgacgcgctataccgttggatcaagtcggtagacccgtcgcgtccggtacaatacgaaggtggcggtgcggacacgacggcgacggacatcatctgcccgatgtacgcgcgcgtcgacgaagaccaaccgttcccggcggtaccgaagtggtcgatcaagaagtggctctcgttgccgggtgaaacgcgtccgttgatactttgcgagtacgcgcacgcgatgggcaactcgttgggtgggttcgcgaagtactggcaggcgttccgtcaatacccgcgtctacagggtgggttcgtctgggactgggtagaccaatcgctaatcaagtacgacgagaacggcaacccgtggtcggcgtacggtggggacttcggggacacgccgaacgaccgccaattctgtatgaacggcctagtcttcgcggaccgaacgccgcacccggcgttgacggaggcgaagcatcaacaacaattcttccaattccgtctatcggggcaaacgatcgaggtaacgtcggagtacttgttccggcactcggacaacgagctactacactggatggtagcactagacggcaagccgctagcgtcgggagaagtccctttggacgtcgcgccgcaaggtaagcaactaatcgagctaccggagctaccgcaaccggagtcggcgggtcaactgtggttgacggtccgtgtcgttcaaccgaacgcgacggcgtggtcggaggcgggtcacatctcggcgtggcagcagtggcgtctagcggagaacctctcggtcacgctaccggcggcgtcgcacgcgataccgcatctaacgacgtcggagatggacttctgcatcgagttggggaacaagaggtggcagttcaaccgtcaatcgggattcctatcgcaaatgtggataggtgacaagaagcaactactaacgccgctacgtgatcagttcacgcgtgctccgctagacaacgacataggtgtttcggaggcgacgcgtatagacccgaacgcgtgggtggagcggtggaaggcggcggggcactaccaagcggaggcggcgctactacagtgcacggcggacacgctagcggacgcggtattgatcacgacggcgcacgcgtggcaacaccaggggaagacgctattcatctcgcgtaagacgtaccgtatcgacggttcgggccaaatggcgatcacggtcgacgtagaggtagcgtcggacacgccgcatccggcgcgcatcggtctaaactgccaactagcgcaagtagcggagcgtgtaaactggctagggctagggccgcaagagaactatccggaccgcctaacggcggcgtgcttcgaccgttgggacctaccgctttcggacatgtataccccgtacgtcttcccgtcggagaacgggttgaggtgcgggacgcgcgagctaaactacgggccgcaccagtggcgaggggacttccaattcaacatatcgcgttactcgcaacaacaactaatggagacgtcgcaccgtcacctactacacgcggaggaggggacgtggctaaacatcgacgggttccacatgggcataggtggggacgactcgtggtcgccgtcggtctcggcggagctccaactctcggcgggtcgttaccattaccaactagtttggtgccagaag

SEQ ID NO: 55 - protein, 119 aa, synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 56 - DNA, 378 bp, synthetic construct
  source 1..378: mol_type = other DNA
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagagatctcatatgcatctcgag

SEQ ID NO: 57 - protein, 264 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGGGGSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 58 - DNA, 792 bp, synthetic construct
  source 1..792: mol_type = other DNA
atggccagcaattttacccagttcgtgctggtcgacaacggcgggaccggtgacgttaccgtggcccctagcaatttcgctaacggcgtggctgagtggatctccagcaacagccggagccaagcttacaaggtgacctgtagcgtgcggcagagcagcgcccagaaccggaagtacaccatcaaggtagaagtgcctaaggtggccacacagactgtgggcggtgtcgagctgcccgtggcagcttggaggagctatctgaatatggaactgaccatccccatcttcgccactaacagcgattgtgagctgattgttaaggccatgcaaggcctgctaaaggacgggaaccccattcccagcgccatcgctgccaacagcggcatctacggaggaggcggaagcgctagcaacttcacccagtttgtgctggtggacaacggtggcaccggcgacgtgaccgtggctcccagcaactttgccaacggtgtggccgagtggatcagcagcaattctcggagccaggcctacaaggtcacgtgcagcgtgcgccaaagcagcgctcagaatcggaagtatactatcaaggtggaggtgcccaaagtggccacccagaccgtgggtggcgtggagctgcctgtggccgcttggcgatcatacctgaacatggagctcaccatccctatcttcgcaaccaacagcgactgcgagctgatcgtgaaagccatgcagggcctgctgaaggacggcaatcccatccccagcgctatcgccgccaatagcggaatctac

SEQ ID NO: 59 - protein, 600 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKAIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 60 - DNA, 1800 bp, synthetic construct
  source 1..1800: mol_type = other DNA
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaaggcgataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 61 - protein, 128 aa, synthetic construct
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 62 - DNA, 384 bp, synthetic construct
  source 1..384: mol_type = other DNA
atgagcaagaccatcgtgctgagcgtgggcgaggccaccagaaccctgaccgagatccagagcaccgccgacagacagatcttcgaggagaaggtgggccctctggtgggcagactgagactgaccgccagcctgagacagaacggcgccaagaccgcctacagagtgaacctgaagctggaccaggccgacgtggtggactgcagcaccagcgtgtgcggcgagctgcctaaggtgagatacacccaggtgtggagccacgacgtgaccatcgtggccaacagcaccgaggccagcagaaagagcctgtacgacctgaccaagagcctggtggtgcaggccaccagcgaggacctggtggtgaacctggtgcctctgggcaga

SEQ ID NO: 63 - protein, 667 aa, synthetic construct
MGSSHHHHHHSQDLEVLFQGPHMGRSRLLEDFRNNRYPNLQLREIAGHIMEFSQDQHGSRFIQLKLERATPAERQLVFNEILQAAYQLMVDVFGSYVIEKFFEFGSLEQKLALAERIRGHVLSLALQMYGSRVIEKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYASYVVEKMIDVAEPGQRKIVMHKIRPHIMEFSQDQHGSRFIELKLERATPAERQLVFNEILQAAYQLMVDVFGCYVIQKFFEFGSLEQKLALAERIRGHVLSLALQMYGSRVIEKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYACYVVQKMIDVAEPGQRKIVMHKIRPHIATLRKYTYGKHILAKLEKYYMKNGVDLG

SEQ ID NO: 64 - DNA, 2001 bp, synthetic construct
  source 1..2001: mol_type = other DNA
atgggcagcagccaccatcaccaccaccacagccaggacctggaggtgctgttccagggcccacacatgggccgtagccggctgctggaggacttccggaacaaccggtaccccaacctgcagctgcgggagatcgccggccacatcatggagttcagccaggaccagcacggcagccgcttcatccagctgaagttggagcgggctacacccgctgagcggcagctggtgttcaacgagatcctgcaggccgcctatcaactgatggtagacgtgttcggcagctacgtgatcgagaagttctttgagttcggcagtctggagcagaagctcgccctggccgaacggatacggggccacgtgcttagcctggccctacagatgtacggcagcagggtgatcgagaaagccctggagttcatccccagcgaccaacagaacgaaatggtgcgggagctcgacggccacgtcctgaagtgcgtgaaggaccagaacggttgccacgtggtgcagaagtgcatcgagtgcgtccagccccagtctctgcagttcattatcgacgccttcaagggccaagtgttcgccctgagtacccacccctacggaagccgggtgatcgaacggattctggagcattgcctgcccgatcagaccctgcccatcctggaggagctgcatcaacacaccgaacagcttgtgcaagaccagtacggctgctacgtgatccagcacgtgttggagcacggacggcccgaggacaagagcaagattgtggccgagatcaggggtaacgtgctggtgctgtctcagcacaaattcgccagcaacgtggtagagaagtgcgtgacccacgccagccggactgaacgggccgtgctgatcgacgaggtgtgcactatgaacgacgggccccactctgccctgtacacaatgatgaaagaccaatacgccagctacgtggtggagaagatgatcgacgtggccgagccaggccaacggaagatcgtgatgcataagatccggcctcatatcatggagtttagccaggatcaacacggcagccggttcatcgagctgaagctggaacgggccacccctgccgaacggcagctggtctttaacgagatactgcaagccgcctaccagctgatggtggacgtgtttggttgctacgttatccagaagttcttcgagttcggaagcctggagcagaaactggccctggccgagcgtatccggggccacgttctgagcttagccctgcagatgtacgggagccgggttatcgagaaggccctggaattcatccctagcgatcagcagaacgagatggtcagagagctggacggccacgtgctcaagtgcgtgaaagaccagaacggctgccacgtggtacagaagtgtatcgagtgtgtgcagccccagagcctccagtttatcattgacgcctttaagggccaggtgtttgccctgagcacacacccctacggcagccgggtaatcgagcggatcctggagcactgcctgcccgaccagaccttgcccatcctcgaggaactgcaccagcacaccgagcagctggtgcaggaccaatacggctgttacgtcatccagcacgttctggagcacggccggcctgaggacaagtctaagatcgtggccgaaatccggggcaacgtgttggtgctgagccagcacaagttcgcctcaaacgtcgtggagaagtgtgtgactcacgcctctcggaccgagcgggcagtgctgattgacgaggtgtgtaccatgaacgacggcccacacagcgccctgtacaccatgatgaaggaccagtacgcctgctacgttgtgcagaagatgattgacgtggccgaacccggtcagcgcaagattgtgatgcacaagatccggccccacatcgccaccctgcggaagtacacctacggcaagcacatcctggccaagctggagaagtactacatgaagaacggcgtggacctgggc

SEQ ID NO: 65 - protein, 667 aa, synthetic construct
MGSSHHHHHHSQDLEVLFQGPHMGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGNRVIQKALEFIPSDQQNEMVRELDGHVLKCVKDQNGCHVVQKCIECVQPQSLQFIIDAFKGQVFALSTHPYGNRVIQRILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIMEFSQDQHGNRFIQLKLERATPAERQLVFNEILQAAYQLMVDVFGNYVIQKFFEFGSLEQKLALAERIRGHVLSLALQMYGNRVIQKALEFIPSDQQNEMVRELDGHVLKCVKDQNGNHVVQKCIECVQPQSLQFIIDAFKGQVFALSTHPYGNRVIQRILEHCLPDQTLPILEELHQHTEQLVQDQYGNYVIQHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 66 - DNA, 2001 bp, synthetic construct
  source 1..2001: mol_type = other DNA
atgggcagctcccatcaccaccaccatcattcgcaggaccttgaagtcctgttccaaggtcctcatatgggtagatcaaggcttttggaagattttcgaaataaccggtatcctaacctgcagctcagggagatagcaggacacatcatggagttcagccaggaccaacacggaagcaggtttatccaactgaaactcgagagagcaacgcccgcagagcgtcagctggtgtttaacgagatcctgcaggcagcctaccagctgatggttgacgtgttcggtaactacgtaattcagaagttctttgagttcggttctctggaacagaaactcgctctggctgagcgaattcggggtcacgtgctctcactggcgctccagatgtacggcaatagagtgattcagaaggctctggaatttattccatcggaccagcagaacgaaatggtacgagaactggacggtcacgtccttaagtgtgtcaaagatcagaacggctgtcacgtagtacagaagtgcatcgagtgtgtgcagccgcagagcctccagtttatcatcgacgcctttaaagggcaggtcttcgccttgtccacccatccgtacggaaaccgagtcattcagcgcatcctggaacattgcctcccggatcagaccctgccgattctggaggagcttcaccagcacaccgagcaactagtgcaggatcagtacggctgttacgtgatacagcacgtcctggagcacggtagaccagaggacaaatcgaagattgtggccgaaatcagaggaaacgtgctggtcctttcccagcacaagttcgcttccaacgtggtggagaagtgcgtgacacacgcaagccgcaccgagagggcagtcttaatcgacgaagtgtgcacaatgaacgacggtccacactctgccttatacacaatgatgaaggaccagtacgcaaactacgtggtgcagaagatgattgacgtagctgagcctggccagcgaaagattgtcatgcataagatcaggcctcatattatggagttttctcaggatcaacacggcaatcggttcatccaattaaagctggagagggccacacctgcggagaggcagttggtctttaacgaaattctacaagccgcttaccaattaatggtggacgtctttggcaactacgtgatccagaaattcttcgagtttggaagtcttgaacagaagctggcgctggctgaaagaatcagaggtcacgtcctcagcctggcattgcaaatgtacggaaatcgagtcatccagaaagcccttgagtttatccccagcgatcaacagaacgagatggttcgcgaattggacgggcacgtgctgaagtgtgttaaggaccagaacgggaaccacgtcgtgcagaagtgcatagagtgcgtccagccacagtccctgcagtttattattgacgcatttaagggacaagtgttcgccttatctacccacccttacggcaatagggtcattcagaggatcttggaacactgtcttcccgaccagacactgccaatattggaagaactgcatcaacataccgaacagctcgtacaagatcagtacggtaattacgtcattcaacacgttctcgaacacggaaggcccgaagacaagtctaagatcgtcgcagagatacgtgggaacgtcctcgtcttaagtcagcataagttcgcaaacaacgttgtgcagaagtgtgtaacacacgcgagcagaacagagagagccgtgctgatcgacgaggtgtgtaccatgaacgacgggccacacagcgccctctacaccatgatgaaagaccaatacgctaactacgtggttcagaagatgatcgacgtcgccgaaccaggtcaacggaagatcgtgatgcacaagatcaggccacacatagccaccctgagaaagtatacgtacggtaaacatatcctggcaaagttagagaagtactatatgaagaacggcgtggacttagga

SEQ ID NO: 67 - protein, 23 aa, synthetic construct
MGNARTRRRERRAEKQAQWKAAN

SEQ ID NO: 68 - DNA, 69 bp, synthetic construct
  source 1..69: mol_type = other DNA
atgggcaacgcccggaccaggagaagagagcggcgggccgagaagcaggcccagtggaaggccgccaac

SEQ ID NO: 69 - protein, 101 aa, synthetic construct
MAVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTF

SEQ ID NO: 70 - DNA, 303 bp, synthetic construct
  source 1..303: mol_type = other DNA
atggccgtgcctgagaccagacctaaccacaccatctacatcaacaacctgaacgagaagatcaagaaggacgagctgaagaagagcctgtacgccatcttcagccagttcggccagatcctggacatcctggtgagcagaagcctgaagatgagaggccaggccttcgtgatcttcaaggaggtgagcagcgccaccaacgccctgagaagcatgcagggcttccctttctacgacaagcctatgagaatccagtacgccaagaccgacagcgacatcatcgccaagatgaagggcaccttc

SEQ ID NO: 71 - protein, 128 aa, synthetic construct
MTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 72 - DNA, 384 bp, synthetic construct
  source 1..384: mol_type = other DNA
atgaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 73 - protein, 582 aa, synthetic construct
METESGNQEKVMEEESTEKKKEVEKKKRSRVKQVLADIAKQVDFWFGDANLHKDRFLREQIEKSRDGYVDISLLVSFNKMKKLTTDGKLIARALRSSAVVELDLEGTRIRRKKPLGERPKDEDERTVYVELLPKNVNHSWIERVFGKCGNVVYISIPHYKSTGDPKGFAFVEFETKEQAAKAIEFLNNPPEEAPRKPGIFPKTVKNKPIPALRVVEEKKKKKKKKGRMKKEDNIQAKEENMDTSNTSISKMKRSRPTSEGSDIESTEPQKQCSKKKKKRDRVEASSLPEVRTGKRKRSSSEDAESLAPRSKVKKIIQKDIIKEASEASKENRDIEISTEEEKDTGDLKDSSLLKTKRKHKKKHKERHKMGEEVIPLRVLSKSEWMDLKKEYLALQKASMASLKKTISQIKSESEMETDSGVPQNTGMKNEKTANREECRTQEKVNATGPQFVSGVIVKIISTEPLPGRKQVRDTLAAISEVLYVDLLEGDTECHARFKTPEDAQAVINAYTEINKKHCWKLEILSGDHEQRYWQKILVDRQAKLNQPREKKRGTEKLITKAEKIRLAKTQQASKHIRFSEYD

SEQ ID NO: 74 - DNA, 1746 bp, synthetic construct
  source 1..1746: mol_type = other DNA
atggagaccgagagcggcaaccaggagaaggtgatggaggaggagagcaccgagaagaagaaggaggtggagaagaagaagagaagcagagtgaagcaggtgctggccgacatcgccaagcaggtggacttctggttcggcgacgccaacctgcacaaggacagattcctgagagagcagatcgagaagagcagagacggctacgtggacatcagcctgctggtgagcttcaacaagatgaagaagctgaccaccgacggcaagctgatcgccagagccctgagaagcagcgccgtggtggagctggacctggagggcaccagaatcagaagaaagaagcctctgggcgagagacctaaggacgaggacgagagaaccgtgtacgtggagctgctgcctaagaacgtgaaccacagctggatcgagagagtgttcggcaagtgcggcaacgtggtgtacatcagcatccctcactacaagagcaccggcgaccctaagggcttcgccttcgtggagttcgagaccaaggagcaggccgccaaggccatcgagttcctgaacaaccctcctgaggaggcccctagaaagcctggcatcttccctaagaccgtgaagaacaagcctatccctgccctgagagtggtggaggagaagaagaagaagaagaagaagaagggcagaatgaagaaggaggacaacatccaggccaaggaggagaacatggacaccagcaacaccagcatcagcaagatgaagagaagcagacctaccagcgagggcagcgacatcgagagcaccgagcctcagaagcagtgcagcaagaagaagaagaagagagacagagtggaggccagcagcctgcctgaggtgagaaccggcaagagaaagagaagcagcagcgaggacgccgagagcctggcccctagaagcaaggtgaagaagatcatccagaaggacatcatcaaggaggccagcgaggccagcaaggagaacagagacatcgagatcagcaccgaggaggagaaggacaccggcgacctgaaggacagcagcctgctgaagaccaagagaaagcacaagaagaagcacaaggagagacacaagatgggcgaggaggtgatccctctgagagtgctgagcaagagcgagtggatggacctgaagaaggagtacctggccctgcagaaggccagcatggccagcctgaagaagaccatcagccagatcaagagcgagagcgagatggagaccgacagcggcgtgcctcagaacaccggcatgaagaacgagaagaccgccaacagagaggagtgcagaacccaggagaaggtgaacgccaccggccctcagttcgtgagcggcgtgatcgtgaagatcatcagcaccgagcctctgcctggcagaaagcaggtgagagacaccctggccgccatcagcgaggtgctgtacgtggacctgctggagggcgacaccgagtgccacgccagattcaagacccctgaggacgcccaggccgtgatcaacgcctacaccgagatcaacaagaagcactgctggaagctggagatcctgagcggcgaccacgagcagagatactggcagaagatcctggtggacagacaggccaagctgaaccagcctagagagaagaagagaggcaccgagaagctgatcaccaaggccgagaagatcagactggccaagacccagcaggccagcaagcacatcagattcagcgagtacgac

SEQ ID NO: 75-148 - skipped (74 entries)

SEQ ID NO: 149 - DNA, 100 bp, synthetic construct
  source 1..100: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 150 - DNA, 120 bp, synthetic construct
  source 1..120: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - DNA, 357 bp, synthetic construct
  source 1..357: mol_type = other DNA
atgtacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaag

SEQ ID NO: 152 - DNA, 399 bp, synthetic construct
  source 1..399: mol_type = other DNA
atgtacgtcagattcgaggtcccagaagatatgcagaacgaggcattatctctgctcgagaaggtaagagaatcagggaaagtcaagaagggaaccaacgaaacaactaaagcagttgaacgcggattagccaaattggtgtacattgccgaagacgttgatccacctgaaattgtggcgcatttacctctactttgcgaggagaagaacgtgccgtatatttacgtgaagtcaaagaacgatctgggtagagcagttgggatcgaagtaccttgtgcaagtgctgccattatcaacgaaggcgaactcaggaaagaacttggctccttagtagagaagatcaaagggctgcagaaaggtaagcctatccccaatccactacttggactagattctacc

SEQ ID NO: 153 - DNA, 399 bp, synthetic construct
  source 1..399: mol_type = other DNA
atgtacgttagatttgaagtaccagaagatatgcagaacgaagctttatctttattggagaaagtcagagaaagtggtaaagttaagaagggaacaaacgagacaactaaagctgttgagaggggattagcaaagttagtttatattgctgaagacgtggatcctccagaaattgttgcacatttgcctttactttgtgaagagaagaacgttccttatatttacgtgaaatctaagaacgatttaggtagagctgttggcattgaagttccctgtgcatccgctgctataattaacgaaggagaattgagaaaggaacttggaagtttagttgagaagattaaaggattgcagaagggtaaaccaataccaaaccctttattaggtcttgattctacc

SEQ ID NO: 154 - DNA, 123 bp, synthetic construct
  source 1..123: mol_type = other DNA
atgagccacggctttcctcctgaggtggaggagcaggacgacggcaccctgcccatgagctgcgcccaggagagcggcatggaccggcatcccgccgcctgtgcctctgcccggatcaacgtg

SEQ ID NO: 155 - DNA, 243 bp, synthetic construct
  source 1..243: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggccgagccagc

SEQ ID NO: 156 - DNA, 243 bp, synthetic construct
  source 1..243: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcgacgccagc

SEQ ID NO: 157 - DNA, 243 bp, synthetic construct
  source 1..243: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcaaggccagc

SEQ ID NO: 158 - DNA, 243 bp, synthetic construct
  source 1..243: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcatggccagc

SEQ ID NO: 159 - DNA, 282 bp, synthetic construct
  source 1..282: mol_type = other DNA
ctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgag

SEQ ID NO: 160 - DNA, 729 bp, synthetic construct
  source 1..729: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtggtggccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtagtggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtcgtcgccagcgccagc

SEQ ID NO: 161 - DNA, 729 bp, synthetic construct
  source 1..729: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtgcgagccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtacgggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtccgtgccagcgccagc

SEQ ID NO: 162 - DNA, 852 bp, synthetic construct
  source 1..852: mol_type = other DNA
atggtgtccaagggtgaggaattgtttaccggggtggtgcctattctcgtcgaacttgacggggatgtgaatggacacaagttttcggtatccggagaaggagagggtgacgccacatacggaaagcttacactcaaattcatctgtacgacggggaaactgcccgtaccctggcctacgctcgtaaccacgctgacttatggagtgcagtgctttagcagataccccgaccatatgaagcagcacgacttcttcaagtcggcgatgcccgaggggtacgtgcaagagaggaccattttcttcaaagacgatggcaattacaaaacacgcgcagaagtcaagtttgagggcgatactctggtcaatcggatcgaattgaagggaatcgatttcaaagaagatggaaacatccttggccataagctcgagtacaactataactcgcataatgtctatatcatggctgacaagcagaaaaacggtatcaaagtcaactttaagatccgacacaatattgaggacggttcggtgcagcttgcggaccactatcaacagaatacgccgattggggatggtccggtccttttgccggataaccattatctctcaacccagtcagccctgagcaaagatccaaacgagaagagggaccacatggtcttgctcgaattcgtgacagcggcagggatcactctgggaatggacgagttgtacaagagatctcgagatatcagccatggcttcccgccggcggtggcggcgcaggatgatggcacgctgcccatgtcttgtgcccaggagagcgggatggaccgtcaccctgcagcctgtgcttctgctaggatcaatgtg

SEQ ID NO: 163 - DNA, 852 bp, synthetic construct
  source 1..852: mol_type = other DNA
atggtgagcaagggcgaggagctgttcaccggcgtggtgcccatcctggtggagctggacggcgacgtgaacggccacaagttcagcgtgtccggcgagggcgagggcgacgccacctacggcaagctgaccctgaagttcatctgcaccaccgggaagctgcccgtgccctggcccaccctggtgaccaccctgacctacggggtgcagtgcttcagcaggtaccccgaccacatgaagcagcacgacttcttcaagagcgccatgcccgagggctacgtgcaggagcgcaccatcttcttcaaggacgacgggaactacaagacccgcgccgaggtgaagttcgagggggacaccctggtgaaccggatcgagctgaaggggatcgacttcaaggaggacggcaacatcctggggcacaagctggagtacaactacaactcccacaacgtgtacatcatggccgacaagcagaagaacggcatcaaggtgaacttcaagatccggcacaacatcgaggacggcagcgtgcagctggccgaccactaccagcagaacacccccatcggcgacggccccgtgctgctgcccgacaaccactacctgagcacccagagcgccctgagcaaggaccccaacgagaagcgggaccacatggtgctgctggagttcgtgaccgccgccggcatcaccctgggcatggacgagctgtacaagcgctcccgggacatctcccacggcttcccacccgccgtggccgctcaggacgacggcaccctgcccatgtcctgcgctcaggagagcgggatggaccgccaccccgccgcctgcgcctccgcccggatcaacgtg

SEQ ID NO: 164 - DNA, 708 bp, synthetic construct
  source 1..708: mol_type = other DNA
atggtatccaagggggaggaggacaacatggcgatcatcaaggagttcatgcgattcaaggtgcacatggaaggttcggtcaacggacacgaatttgaaatcgaaggagagggtgaaggaaggccctatgaagggacacagaccgcgaaactcaaggtcacgaaagggggaccacttcctttcgcctgggacattctttcgccccagtttatgtacgggtccaaagcatatgtgaagcatcccgccgatattcctgactatctgaaactcagctttcccgagggattcaagtgggagcgggtcatgaactttgaggacgggggtgtagtcaccgtaacccaagactcaagcctccaagacggcgagttcatctacaaggtcaaactgcgggggactaactttccgtcggatgggccggtgatgcagaagaaaacgatgggatgggaagcgtcatcggagaggatgtacccagaagatggtgcattgaagggggagatcaagcagagactgaagttgaaagatgggggacattatgatgccgaggtgaaaacgacatacaaagcgaaaaagccggtgcagcttcccggagcgtataatgtgaatatcaagttggatattacttcacacaatgaggactacacaattgtcgaacagtacgaacgcgctgagggtagacactcgacgggaggcatggacgagttgtacaaa

SEQ ID NO: 165 - DNA, 579 bp, synthetic construct
  source 1..579: mol_type = other DNA
atgggagtgcacgagtgtcccgcgtggttgtggttgctgctgtcgctcttgagcctcccactgggactgcctgtgctgggggcaccacccagattgatctgcgactcacgggtacttgagaggtaccttcttgaagccaaagaagccgaaaacatcacaaccggatgcgccgagcactgctccctcaatgagaacattactgtaccggatacaaaggtcaatttctatgcatggaagagaatggaagtaggacagcaggccgtcgaagtgtggcaggggctcgcgcttttgtcggaggcggtgttgcggggtcaggccctcctcgtcaactcatcacagccgtgggagcccctccaacttcatgtcgataaagcggtgtcggggctccgcagcttgacgacgttgcttcgggctctgggcgcacaaaaggaggctatttcgccgcctgacgcggcctccgcggcacccctccgaacgatcaccgcggacacgtttaggaagctttttagagtgtacagcaatttcctccgcggaaagctgaaattgtatactggtgaagcgtgtaggacaggggatcgc

SEQ ID NO: 166 - DNA, 1392 bp, synthetic construct
  source 1..1392: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtgcgagccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtacgggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtccgtgccagcgccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 167 - DNA, 1392 bp, synthetic construct
  source 1..1392: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtggtggccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtagtggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtcgtcgccagcgccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 168 - DNA, 906 bp, synthetic construct
  source 1..906: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggccgagccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 169 - DNA, 906 bp, synthetic construct
  source 1..906: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcaaggccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 170 - DNA, 786 bp, synthetic construct
  source 1..786: mol_type = other DNA
atgagccacggctttcctcctgaggtggaggagcaggacgacggcaccctgcccatgagctgcgcccaggagagcggcatggaccggcatcccgccgcctgtgcctctgcccggatcaacgtgtacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 171 - DNA, 906 bp, synthetic construct
  source 1..906: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcgacgccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 172 - DNA, 906 bp, synthetic construct
  source 1..906: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcatggccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagctgaagtacaagcccctgttgaagatcagcaagaactgcgaggccgccatcctgcgggccagcaagacccggctgaacaccatccgggcctacggcagcaccgtgcccaagagcaagagcttcgagcaggacagccggaagcggacccagagctggaccgccctgcgggtgggtgcaatcctggccgccaccagcagcgtggcctacctgaactggcacaacggccagatcgacaacgagcccaagctggacatgaacaagcagaagattagccccgccgagatccccaacccactgctgggcctggac

SEQ ID NO: 173 - DNA, 1110 bp, synthetic construct
  source 1..1110: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtgcgagccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtacgggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtccgtgccagcgccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 174 - DNA, 1110 bp, synthetic construct
  source 1..1110: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtggtggccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtagtggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtcgtcgccagcgccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 175 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggccgagccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 176 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcaaggccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 177 - DNA, 504 bp, synthetic construct
  source 1..504: mol_type = other DNA
atgagccacggctttcctcctgaggtggaggagcaggacgacggcaccctgcccatgagctgcgcccaggagagcggcatggaccggcatcccgccgcctgtgcctctgcccggatcaacgtgtacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 178 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcgacgccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 179 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcatggccagctacgtgcggttcgaggtgcccgaggacatgcagaacgaggccctgagcctgctggagaaggtgcgggagagcggcaaggtgaagaagggcaccaacgagaccaccaaggccgtggagcggggtctggctaagctggtgtacatcgccgaggacgtggaccctcccgagatcgtggcccacctgcccctgctgtgcgaggagaagaacgtgccctacatctacgtgaagagtaagaacgaccttggccgggccgtgggcatcgaggtcccctgcgccagcgctgccatcatcaacgagggcgagctgcggaaggagctgggcagcctggtggagaagatcaagggcctgcagaagatccccaacccactgctgggcctggac

SEQ ID NO: 180 - DNA, 1110 bp, synthetic construct
  source 1..1110: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtgcgagccagcgcaagccagatctttgttaagaccctgaccgggaagacaatcactctggaagtggagcccagcgacactatcgagaacgttaaagctaagattcaagacaaggaggggattccacctgaccagcaacggctgattttcgccggcaaacagctggaagacggccggacactgagcgactacaacattcagaaagagtccaccctgcatctggtgcttcggctgcggggtgtacgggctagcgccagtcagatcttcgtgaagaccctcaccggcaagacgattaccttagaggtggagcctagcgataccatagagaacgtgaaggctaagatccaggataaggagggcatccctcctgatcaacaacggttaatctttgctggcaagcagctggaggacggacggaccctgagcgattacaatatccagaaggagagcaccttgcacctcgtgctgcggctgagaggcgtccgtgccagcgccagcaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 181 - DNA, 630 bp, synthetic construct
  source 1..630: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtgtgcgagccagcgcaagcaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 182 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggccgagccagcaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 183 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcaaggccagcaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 184 - DNA, 624 bp, synthetic construct
  source 1..624: mol_type = other DNA
atgaccagccagattttcgtcaagacactgacaggcaagaccatcaccctggaggtggaaccctccgacaccattgagaacgtcaaggccaagatacaggacaaggaaggcatacctcccgatcagcagaggctcatcttcgcaggcaagcaactcgaggacggcaggaccctgtctgactataacatccagaaagaaagcactctgcacctggttctgcggctccgcggtggcatggccagcaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 185 - protein, 119 aa, synthetic construct; identical to SEQ ID NO: 55

SEQ ID NO: 186 - protein, 41 aa, synthetic construct
MSHGFPPEVEEQDDGTLPMSCAQESGMDRHPAACASARINV

SEQ ID NO: 187 - protein, 81 aa, synthetic construct
MTSQIFVKTLTGKTITLEVEPSDTIENVKAKIQDKEGIPPDQQRLIFAGKQLEDGRTLSDYNIQKESTLHLVLRLRGGRAS

SEQ ID NO: 188 - protein, 81 aa, synthetic construct
MTSQIFVKTLTGKTITLEVEPSDTIENVKAKIQDKEGIPPDQQRLIFAGKQLEDGRTLSDYNIQKESTLHLVLRLRGGDAS

SEQ ID NO: 189 - protein, 81 aa, synthetic construct
MTSQIFVKTLTGKTITLEVEPSDTIENVKAKIQDKEGIPPDQQRLIFAGKQLEDGRTLSDYNIQKESTLHLVLRLRGGKAS

SEQ ID NO: 190 - protein, 81 aa, synthetic construct
MTSQIFVKTLTGKTITLEVEPSDTIENVKAKIQDKEGIPPDQQRLIFAGKQLEDGRTLSDYNIQKESTLHLVLRLRGGMAS

SEQ ID NO: 191 - protein, 94 aa, synthetic construct
LKYKPLLKISKNCEAAILRASKTRLNTIRAYGSTVPKSKSFEQDSRKRTQSWTALRVGAILAATSSVAYLNWHNGQIDNEPKLDMNKQKISPAE

SEQ ID NO: 192 - protein, 243 aa, synthetic construct
MTSQIFVKTLTGKTITLEVEPSDTIENVKAKIQDKEGIPPDQQRLIFAGKQLEDGRTLSDYNIQKESTLHLVLRLRGVVASASQIFVKTLTGKTITLEVEPSDTIENVKAKIQDKEGIPPDQQRLIFAGKQLEDGRTLSDYNIQKESTLHLVLRLRGVVASASQIFVKTLTGKTITLEVEPSDTIENVKAKIQDKEGIPPDQQRLIFAGKQLEDGRTLSDYNIQKESTLHLVLRLRGVVASAS

SEQ ID NO: 193 - protein, 243 aa, synthetic construct
MTSQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASAS

SEQ ID NO: 194 - protein, 284 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSRDISHGFPPAVAAQDDGTLPMSCAQESGMDRHPAACASARINV

SEQ ID NO: 195 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 196 - protein, 193 aa, synthetic construct
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 197 - protein, 464 aa, synthetic construct
MTSQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 198 - protein, 464 aa, synthetic construct
MTSQIFVKTLTGKTITLEVEPSDTIENVKAKIQDKEGIPPDQQRLIFAGKQLEDGRTLSDYNIQKESTLHLVLRLRGVVASASQIFVKTLTGKTITLEVEPSDTIENVKAKIQDKEGIPPDQQRLIFAGKQLEDGRTLSDYNIQKESTLHLVLRLRGVVASASQIFVKTLTGKTITLEVEPSDTIENVKAKIQDKEGIPPDQQRLIFAGKQLEDGRTLSDYNIQKESTLHLVLRLRGVVASASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 199 - protein, 302 aa, synthetic construct
MTSQIFVKTLTGKTITLEVEPSDTIENVKAKIQDKEGIPPDQQRLIFAGKQLEDGRTLSDYNIQKESTLHLVLRLRGGRASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 200 - protein, 302 aa, synthetic construct
MTSQIFVKTLTGKTITLEVEPSDTIENVKAKIQDKEGIPPDQQRLIFAGKQLEDGRTLSDYNIQKESTLHLVLRLRGGKASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 201 - protein, 262 aa, synthetic construct
MSHGFPPEVEEQDDGTLPMSCAQESGMDRHPAACASARINVYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 202 - protein, 302 aa, synthetic construct
MTSQIFVKTLTGKTITLEVEPSDTIENVKAKIQDKEGIPPDQQRLIFAGKQLEDGRTLSDYNIQKESTLHLVLRLRGGDASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 203 - protein, 302 aa, synthetic construct
MTSQIFVKTLTGKTITLEVEPSDTIENVKAKIQDKEGIPPDQQRLIFAGKQLEDGRTLSDYNIQKESTLHLVLRLRGGMASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKLKYKPLLKISKNCEAAILRASKTRLNTIRAYGSTVPKSKSFEQDSRKRTQSWTALRVGAILAATSSVAYLNWHNGQIDNEPKLDMNKQKISPAEIPNPLLGLD

SEQ ID NO: 204 - protein, 370 aa, synthetic construct
MTSQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 205 - protein, 370 aa, synthetic construct
MTSQIFVKTLTGKTITLEVEPSDTIENVKAKIQDKEGIPPDQQRLIFAGKQLEDGRTLSDYNIQKESTLHLVLRLRGVVASASQIFVKTLTGKTITLEVEPSDTIENVKAKIQDKEGIPPDQQRLIFAGKQLEDGRTLSDYNIQKESTLHLVLRLRGVVASASQIFVKTLTGKTITLEVEPSDTIENVKAKIQDKEGIPPDQQRLIFAGKQLEDGRTLSDYNIQKESTLHLVLRLRGVVASASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 206 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGRASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 207 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGKASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 208 - protein, 168 aa, synthetic construct
MSHGFPPEVEEQDDGTLPMSCAQESGMDRHPAACASARINVYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 209 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGDASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 210 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGMASYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKIPNPLLGLD

SEQ ID NO: 211 - protein, 370 aa, synthetic construct
MTSQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASASTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 212 - protein, 210 aa, synthetic construct
MTSQIFVKTLTGKTITLEVEPSDTIENVKAKIQDKEGIPPDQQRLIFAGKQLEDGRTLSDYNIQKESTLHLVLRLRGVRASASTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 213 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGRASTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 214 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGKASTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 215 - protein, 208 aa, synthetic construct
MTSQIFVKTLTGKTITLEVEPSDTIENVKAKIQDKEGIPPDQQRLIFAGKQLEDGRTLSDYNIQKESTLHLVLRLRGGMASTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

(19) United States
(12) Patent Application Publication
(10) Pub. No.: US 2026/0035707 A1
(43) Pub. Date: Feb. 5, 2026

(54) ENGINEERED POLYNUCLEOTIDES FOR TEMPORAL CONTROL OF EXPRESSION

(71) Applicant: ModernaTX, Inc., Cambridge, MA (US)

(72) Inventor: Ruchi Jain, Chestnut Hill, MA (US)

(21) Appl. No.: 18/998,310

(22) PCT Filed: Jul. 24, 2023
(86) PCT No.: PCT/US2023/070813

§ 371 Date: Jan. 24, 2025

Related U.S. Application Data
(60) Provisional application No. 63/392,209, filed on Jul. 26, 2022.

(51) Int. Cl. C12N 15/63; C07K 14/47; C12N 15/62; C12N 15/85

(52) U.S. Cl. CPC C12N 15/635; C07K 14/4703; C12N 15/62; C12N 15/85; C07K 2319/00; C12N 2840/50

(57) ABSTRACT

The disclosure features compositions, systems, and uses thereof, comprising polynucleotide(s) encoding target molecule(s), and polynucleotide(s) encoding repressor(s) or timer(s). The repressor(s) or timer(s) delay expression of the target molecule(s).

### CROSS-REFERENCE TO RELATED APPLICATIONS

This application claims priority to U.S. Patent Application No. 63/392,209, filed on Jul. 26, 2022, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Jul. 18, 2023, is named 45817-0117WO1.xml and is 229,314 bytes in size.

### BACKGROUND

One of the key issues in successfully implementing mRNA therapies in the clinical setting is to be able to regulate mRNA expression very tightly and consistently as and when it is needed. Controlling the timing of mRNA expression is a challenge that would benefit multiple modalities such as facilitating correct pairing of antibodies by staggering mRNAs encoding for different antibody pairs, allowing mRNAs to reach appropriate locations before making protein, and timing the expression of antigen vs Type 1 interferon induction for optimal T cell response.

Thus, there is a need to temporally control the expression of mRNA, such that it is expressed as desired and when desired in order to increase therapeutic efficacy and reduce untoward side effects. This would have wide-ranging applications in, for example, protein engineering (e.g., multispecific antibody engineering), immunotherapy and immune-oncology therapeutics, in vivo gene editing, stem cell transplantation, and gene therapies for various diseases.

### SUMMARY

The present disclosure provides, inter alia, compositions, systems, and methods for controlling the timing of mRNA expression.

In one aspect, the disclosure features a composition comprising:
(a) a first polynucleotide comprising (i) a repressor binding element, and (ii) an open reading frame encoding a first polypeptide; and
(b) a second polynucleotide comprising a sequence encoding a fusion polypeptide comprising (i) a repressor that binds to the repressor binding element, and (ii) a destabilization domain,
wherein binding of the repressor to the repressor binding element reduces translation of the first polypeptide from the first polynucleotide, and wherein the destabilization domain enhances degradation of the repressor.

In some embodiments, the destabilization domain comprises a ubiquitin tag.

In some embodiments, the first polynucleotide is an mRNA and comprises a polyA tail.

In some embodiments, the first polynucleotide is a DNA.

In some embodiments, the second polynucleotide is an mRNA and comprises a polyA tail.

In some embodiments, the second polynucleotide is a DNA.

In some embodiments, the second polynucleotide is a circular RNA.

In some embodiments, the first and second polynucleotides are DNA and are encoded in a single plasmid.

In some embodiments, the repressor binding element comprises a kink-turn forming sequence.

In some embodiments, the repressor binding element is selected from the group consisting of PRE, PRE2, MS2, PP7, BoxB, U1A hairpin, and 7SK.

In some embodiments, the repressor is the 50S ribosomal L7Ae protein, the 15.5 kd repressor, Pumilio and FBF (PUF) protein, PUF2 protein, MBP-LacZ, MBP, PCP, Lambda N, U1A, LARP7, Snu13, or variants thereof.

In some embodiments, the ubiquitin tag is 3XUbVR, 3XUbVV, UbR, UbK, PEST, UbD, or UbM.

In some embodiments, the fusion polypeptide further comprises a cytochrome b2 region.

In some embodiments, the first polypeptide is a secreted protein, a membrane-bound protein, or an intercellular protein.

In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, (a) and (b) are in separate dosage forms packaged together.

In some embodiments, (a) and (b) are in a unit dosage form.

In another aspect, the disclosure features a method of expressing a first polypeptide in a cell, the method comprising contacting the cell with a composition described herein.

In another aspect, the disclosure features a method of expressing a first polypeptide in a cell, the method comprising contacting the cell with:
(a) a first polynucleotide comprising (i) a repressor binding element, and (ii) an open reading frame encoding a first polypeptide; and
(b) a second polynucleotide comprising a sequence encoding a fusion polypeptide comprising (i) a repressor that binds to the repressor binding element, and (ii) a destabilization domain,
wherein binding of the repressor to the repressor binding element reduces translation of the first polypeptide from the first polynucleotide, and wherein the destabilization domain enhances degradation of the repressor.

In another aspect, the disclosure features a method of expressing a first polypeptide in a subject, the method comprising administering to the subject:
(a) a first polynucleotide comprising (i) a repressor binding element, and (ii) an open reading frame encoding a first polypeptide; and
(b) a second polynucleotide comprising a sequence encoding a fusion polypeptide comprising (i) a repressor that binds to the repressor binding element, and (ii) a destabilization domain,
wherein binding of the repressor to the repressor binding element reduces translation of the first polypeptide from the first polynucleotide, and wherein the destabilization domain enhances degradation of the repressor.

In some embodiments, the cell is a liver cell, a muscle cell, immune cell or a neuron.

In some embodiments, the first polynucleotide is a DNA or an mRNA and the second polynucleotide is a DNA or an mRNA.

In another aspect, the disclosure features a composition comprising
(a) a first polynucleotide comprising (i) a repressor binding element, and (ii) an open reading frame encoding a first polypeptide;
(b) a second polynucleotide comprising an open reading frame encoding a second polypeptide; and
(c) a third polynucleotide comprising a sequence encoding a fusion polypeptide comprising (i) a repressor that binds to the repressor binding element, and (ii) a destabilization domain; and
wherein binding of the repressor to the repressor binding element reduces translation of the first polypeptide from the first polynucleotide, and wherein the destabilization domain enhances degradation of the repressor.

In another aspect, the disclosure features a composition comprising
(a) a first polynucleotide comprising (i) a first repressor binding element, and (ii) an open reading frame encoding a first polypeptide;
(b) a second polynucleotide comprising (i) a second repressor binding element, and (ii) an open reading frame encoding a second polypeptide;
(c) a third polynucleotide comprising a sequence encoding a first fusion polypeptide comprising (i) a first repressor that binds to the first repressor binding element, and (ii) a first destabilization domain; and
(d) a fourth polynucleotide comprising a sequence encoding a second fusion polypeptide comprising (i) a second repressor that binds to the second repressor binding element, and (ii) a second destabilization domain; and
wherein binding of the first and second repressors to the first and second repressor binding elements respectively reduce translation of the first and second polypeptides from the first and second polynucleotides, and wherein the first and second destabilization domains respectively degrade the first and second repressors at different rates.

In another aspect, the disclosure features a composition comprising
(a) a first polynucleotide comprising (i) a first repressor binding element, and (ii) an open reading frame encoding a first polypeptide;
(b) a second polynucleotide comprising (i) a second repressor binding element, and (ii) an open reading frame encoding a second polypeptide;
(c) a third polynucleotide comprising an open reading frame encoding a third polypeptide;
(d) a fourth polynucleotide comprising a sequence encoding a first fusion polypeptide comprising (i) a first repressor that binds to the first repressor binding element, and (ii) a first destabilization domain; and
(e) a fifth polynucleotide comprising a sequence encoding a second fusion polypeptide comprising (i) a second repressor that binds to the second repressor binding element, and (ii) a second destabilization domain; and
wherein binding of the first and second repressors to the first and second repressor binding elements respectively reduce translation of the first and second polypeptides from the first and second polynucleotides, and wherein the first and second destabilization domains respectively degrade the first and second repressors at different rates.

Other features and advantages of the invention will be apparent from the following detailed description, and from the claims.

### BRIEF DESCRIPTION OF THE DRAWINGS

FIG. 1A provides a schematic of a system in which RNA translation has a delayed onset. RNA #1 undergoes translation into protein #1. RNA #2 undergoes translation into protein #2 with delayed onset due to timer-based repression of translation. FIG. 1B shows a degron attached to L7Ae protein which binds to target RNA. The degron destabilizes the L7Ae protein, thereby limiting its duration of repression of the target RNA.

FIG. 2 is a graph depicting the total green integrated intensity over time when 20 ng 5′kt deg-eGFP and effectors at 1:0.01 Target: Effector moles, were co-transfected in HeLa cells and imaged by Incucyte over 48 h. The destabilization domains tested were 1 (3XUbVR+cb2), 2 (3XUbVV+cb2), 3 (3XUbVV), 4 (3XUbVR), and 5 (L7Ae+UbR, UbK, PEST, UbD, UbM+/1 Cb2).

FIGS. 3A-3C show delayed onset of detectable expression by codelivering Effectors, Filler (EPO) or Timer (3XUbVR-L7Ae), with target 5′kt deg-eGFP RNA in HeLa cells. FIG. 3A are graphs depicting the effect of increasing levels of Timer on timing of expression in HeLa cells. FIG. 3B is a graph depicting deg-eGFP expression levels across different amounts of Timer. FIG. 3C is a graph depicting the correlation of delay interval with fraction of Timer in the system.

FIG. 4A is a graph depicting total green integrated intensity over time from cells transfected with degGFP codelivered with Effectors and filler (EPO), timer (3XUbVR-L7Ae), or repressor (L7Ae) constructs.

FIG. 4B is a graph depicting normalized total green integrated intensity over time from cells transfected with degGFP codelivered with Effectors and filler (EPO), timer (3XUbVR-L7Ae), or repressor (L7Ae) constructs.

FIG. 5A is a graph depicting normalized total green integrated intensity over time from cells transfected with degGFP and effectors and filler or timer constructs, with effectors at 0.05× Target:Effector moles.

FIG. 5B is a graph depicting normalized total green integrated intensity over time from cells transfected with degGFP and effectors and filler or timer constructs, with effectors at 0.1× Target:Effector moles.

FIG. 5C is a graph depicting normalized total green integrated intensity over time from cells transfected with degGFP and effectors and filler or timer constructs, with effectors at 0.2× Target:Effector moles.

FIG. 6A is a graph depicting total green integrated intensity (area under curve, AUC) over time from cells transfected, at various Target: Effector ratios, with filler, timer or repressor. FIG. 6B is a graph depicting fraction of timer vs delay in hours. FIG. 6C is a table depicting timer concentration vs. delay in hours.

FIG. 7 is a table depicting the levels of target protein rescue and delay seen with transfection of the various reporter systems in the indicated cell types.

FIG. 8 is a schematic of a system in which expression of two fluorescent proteins (mCherry and GFP) is staggered by using a tethered timer system.

FIG. 9A is a series of photos of expression over time from HeLa cells transfected with two fluorescent proteins in a tethered Timer system.

FIG. 9B is a graph depicting total integrated intensity over time from primary human hepatocytes transfected with two fluorescent proteins in a tethered Timer system.

FIG. 9C is a graph depicting normalized total integrated intensity over time from primary human hepatocytes transfected with two fluorescent proteins in a tethered Timer system.

FIG. 10A is a graph depicting the total green integrated intensity from cells transfected with GFP and different timer encoding RNAs (all encoding 3×UBVR_L7Ae but made with different coding sequences and/or 5′UTR) vs filler.

FIG. 10B is a graph depicting the normalized total green integrated intensity from cells transfected with GFP and different timer encoding RNAs (all encoding 3×UBVR_L7Ae but made with different coding sequences and/or 5′UTR) vs filler.

FIG. 11A is a graph depicting the confluence percentage over time in cells with different concentrations of Snu13 repressor RNA.

FIG. 11B is a graph depicting the confluence percentage over time in cells with different concentrations of Snu13 repressor RNA or L7Ae repressor RNA.

FIG. 12A is a graph depicting total green integrated intensity over time for a green fluorescent protein encoding RNA co-delivered in HeLa cells with Snu13 (Timer_v2) encoding RNA fused to different degron domains or filler (EPO).

FIG. 12B is a graph depicting total green integrated intensity over time for a green fluorescent protein encoding RNA co-delivered in primary human hepatocytes with Snu13 (Timer_v2) encoding RNA fused to different degron domains or filler (EPO).

FIG. 13A is a graph depicting the normalized total green integrated intensity over time in HeLa cells co-transfected with Snu13 (Timer_v2) design variants or a filler (EPO) and a green fluorescent protein target mRNA.

FIG. 13B is a graph depicting the normalized total green integrated intensity over time in primary hepatocytes co-transfected with Snu13 (Timer_v2) design variants or a filler (EPO) and a green fluorescent protein target mRNA.

FIG. 14A is a graph depicting the area under the curve indicating % rescue and the delay in HeLa cells co-transfected with Snu13 (Timer_v2) design variants or a filler (EPO) and a green fluorescent protein target mRNA.

FIG. 14B is a graph depicting the area under the curve indicating % rescue and the delay for in primary hepatocytes co-transfected with Snu13 (Timer_v2) design variants or a filler (EPO) and a green fluorescent protein target mRNA.

FIG. 15 shows a summary of reporter RNA translation delay and rescue in the indicated cell types over time.

### DETAILED DESCRIPTION

Controlling the timing of mRNA expression is critical in ensuring that therapeutic products are available when they are needed, and to strive for the highest clinical efficacy. The present disclosure is based on the development of a “timer system”, wherein a tethered repressor protein is exploited to achieve a delayed onset of mRNA expression, in which mRNA begins translating after set time intervals. Further, the present disclosure is based on the discovery that various design modifications can be employed for increasing efficacy of the timer system.

Accordingly, disclosed herein are compositions or systems comprising polynucleotide constructs in which the timing of expression of the target mRNA is dependent upon a timer system. The onset of expression of the one or more target mRNAs of the timer systems described herein can be controlled by, for example, changing the degradation of the repressor protein using degrons (and/or mutations) that impact protein stability or changing the amount of repressor protein in the system. The amount of repressor protein can itself be controlled by, for example, changing the amount of mRNA encoding the repressor or changing the open reading frame design principals of the mRNA encoding the repressor.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering one or more polynucleotides of this disclosure to a subject may involve administering a composition (e.g., an LNP including the one or more polynucleotides) to the subject (e.g., by an intravenous, intramuscular, intradermal, pulmonary or subcutaneous route). Administration of a composition (e.g., an LNP) to a mammal or mammalian cell may involve contacting one or more cells with the composition.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. However, in some embodiments the mRNA does not have a polyA tail. In some embodiments, the mRNA is a circular mRNA.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Repressor binding element: As used herein, the term “repressor binding element” or “binding element” refers to a nucleic acid sequence, e.g., a DNA or RNA sequence, which is recognized by a repressor molecule. In an embodiment, the binding element forms a structure, e.g., a three-dimensional structure, e.g., a kink-turn, a loop, a stem or other known structure. Exemplary binding elements are provided in Table 1.

Repressor Molecule: As used herein, the term “repressor molecule” or “repressor” refers to a molecule which binds to, e.g., recognizes, a binding element or a fragment thereof. In an embodiment, the repressor binds to, e.g., recognizes, a sequence, e.g., a DNA or RNA sequence, comprising the binding element, or fragment thereof. In an embodiment, the repressor binds to, e.g., recognizes, a structure comprising a sequence, e.g., a DNA or RNA sequence, comprising the binding element, or fragment thereof. In an embodiment, the repressor comprises an RNA-binding protein or a fragment thereof. Exemplary repressors are provided in Table 1.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

Unsuitable for canonical translation: A polynucleotide (e.g., RNA) that is “unsuitable for canonical translation” is a polynucleotide with a nucleotide modification, sequence modification, and/or a structure that is not suitable for translation. In some embodiments, the modification(s) are at the 5′ end, and/or the 3′ end. In some embodiments, the modification(s) stabilize the polynucleotide. In some embodiments, a polynucleotide that is unsuitable for canonical translation (a) does not have a polyA tail; (b) is circular; (c) has no cap; and/or (d) has no cap and no tail.

### Polynucleotides of Timer Systems

Disclosed herein, inter alia, are timer compositions and systems that encode one or more polypeptides and optionally, one or more repressors or timers. In a dual polynucleotide system, the first polynucleotide of the composition encodes for a polypeptide, that is a target protein. Additionally, the first polynucleotide also has a repressor binding element. In a dual polynucleotide system, the second polynucleotide of the composition encodes a timer protein.

The timer system can have more than two polynucleotides, e.g., three, four, five or more polynucleotides. In such systems, one or more polynucleotides encode one of more target proteins, and one or more polynucleotides encode one or more repressors or timers. The duration of repression of the target RNA by the repressors or timers encoded by the one of more polynucleotides can vary.

For example, in a three polynucleotide timer system, a first polynucleotide can encode a first target RNA and also has a repressor binding element, a second polynucleotide can encode a second target RNA, and a third polynucleotide can encode a timer that binds to the repressor binding element of the first polynucleotide, thereby altering duration of repression of the first target RNA only. Thus, the timing of expression of the first target RNA will be different from that of the second target RNA.

In another example, in a four polynucleotide timer system, a first polynucleotide can encode a first target RNA and also has a first repressor binding element, a second polynucleotide can encode a second target RNA and also has a second repressor binding element, a third polynucleotide can encode a first timer that binds to the first repressor binding element, and a fourth polynucleotide can encode a second timer that binds to the second repressor binding element. In such a system, each timer differentially alters the duration of repression of the first and second target RNA. Thus, the timing of expression of the first target RNA will be different from that of the second target RNA.

In another example, in a five polynucleotide timer system, a first polynucleotide can encode a first target RNA and also has a first repressor binding element, a second polynucleotide can encode a second target RNA and also has a second repressor binding element, a third polynucleotide can encode a third target RNA, a fourth polynucleotide can encode a first timer that binds to the first repressor binding element, and a fifth polynucleotide can encode a second timer that binds to the second repressor binding element. In such a system, each timer differentially alters the duration of repression of the first and second target RNA, and the duration of expression of the third target RNA is unaltered. Thus, the timing of expression of the first, second, and third target RNAs will vary from each other.

The polynucleotide timer systems and its features are discussed below in greater detail.

### Target Polypeptides of Timer Systems

In some embodiments of the timer systems disclosed herein, the target polypeptide(s) (i.e., the polypeptides encoded by the one or more target RNAs) can be, but are not limited to, any of the following: a secreted protein; a membrane-bound protein; or an intercellular protein, or peptides, polypeptides or biologically active fragments thereof.

In some embodiments, the target polypeptide is a secreted protein, or a peptide, a polypeptide or a biologically active fragment thereof. In some embodiments, the secreted protein comprises a cytokine, or a variant or fragment (e.g., a biologically active fragment) thereof. In some embodiments, the secreted protein comprises an antibody or a variant or fragment (e.g., a biologically active fragment) thereof. In some embodiments, the secreted protein comprises an enzyme or a variant or fragment (e.g., a biologically active fragment) thereof. In some embodiments, the secreted protein comprises a hormone or a variant or fragment (e.g., a biologically active fragment) thereof. In some embodiments, the secreted protein comprises a ligand, or a variant or fragment (e.g., a biologically active fragment) thereof. In some embodiments, the secreted protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the secreted protein comprises a growth factor or a component, variant or fragment (e.g., a biologically active fragment) thereof. In some embodiments, the secreted protein comprises an immune modulator, e.g., an immune checkpoint agonist or antagonist.

In some embodiments, the target polypeptide is a membrane-bound protein, or a peptide, a polypeptide or a biologically active fragment thereof. In some embodiments, the membrane-bound protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the membrane-bound protein comprises a ligand, a variant or fragment (e.g., a biologically active fragment) thereof. In some embodiments, the membrane-bound protein comprises a membrane transporter, a variant or fragment (e.g., a biologically active fragment) thereof. In some embodiments, the membrane-bound protein comprises a structural protein, a variant or fragment (e.g., a biologically active fragment) thereof. In some embodiments, the membrane-bound protein comprises an immune modulator, e.g., an immune checkpoint agonist or antagonist.

In some embodiments, the target polypeptide is an intracellular protein, or a peptide, a polypeptide or a biologically active fragment thereof. In some embodiments, the intracellular protein comprises an enzyme, or a variant or fragment (e.g., a biologically active fragment) thereof. In some embodiments, the intracellular protein comprises a hormone, or a variant or fragment (e.g., a biologically active fragment) thereof. In some embodiments, the intracellular protein comprises a cytokine, or a variant or fragment (e.g., a biologically active fragment) thereof. In some embodiments, the intracellular protein comprises a transcription factor, or a variant or fragment (e.g., a biologically active fragment) thereof. In some embodiments, the intracellular protein comprises a nuclease, or a variant or fragment (e.g., a biologically active fragment) thereof. In some embodiments, the intracellular protein comprises comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the intracellular protein comprises a structural protein, or a variant or fragment (e.g., a biologically active fragment) thereof.

In some embodiments, the target polypeptide is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, an immune modulator, or a component, variant or fragment (e.g., a biologically active fragment) thereof.

In some embodiments, the target polypeptide comprises a cytokine, or a variant or fragment (e.g., a biologically active fragment) thereof.

In some embodiments, the target polypeptide comprises an antibody or a variant or fragment (e.g., a biologically active fragment) thereof.

In some embodiments, the target polypeptide comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine.

In some embodiments, the target polypeptide comprises a receptor, or a variant or fragment (e.g., a biologically active fragment) thereof.

In some embodiments, the target polypeptide comprises an enzyme, or a variant or fragment (e.g., a biologically active fragment) thereof.

In some embodiments, the target polypeptide comprises a hormone, or a variant or fragment (e.g., a biologically active fragment) thereof.

In some embodiments, the target polypeptide comprises a growth factor, or a variant or fragment (e.g., a biologically active fragment) thereof.

In some embodiments, the target polypeptide comprises a nuclease, or a variant or fragment (e.g., a biologically active fragment) thereof.

In some embodiments, the target polypeptide comprises a transcription factor, or a variant or fragment (e.g., a biologically active fragment) thereof.

In some embodiments, the target polypeptide comprises a ligand, or a variant or fragment (e.g., a biologically active fragment) thereof.

In some embodiments, the target polypeptide comprises a membrane transporter, or a variant or fragment (e.g., a biologically active fragment) thereof.

In some embodiments, the target polypeptide comprises a structural protein, or a variant or fragment (e.g., a biologically active fragment) thereof.

In some embodiments, the target polypeptide comprises an immune modulator, or a variant or fragment (e.g., a biologically active fragment) thereof. In some embodiments, the immune modulator comprises an immune checkpoint agonist or antagonist.

In some embodiments, the target polypeptide comprises a protein or peptide.

In some embodiments, the target polypeptide comprises a protein associated with gene editing in a cell (an editor protein). For instance, the protein is a Cas nuclease, a zinc finger nuclease, or a homing endonuclease. In some embodiments, the target polypeptide comprises a self-splicing intein, wherein the editor protein is fused to a degron domain that leads to rapid protein degradation.

### Fusion Polypeptides

In some embodiments of the timer systems disclosed herein, one or more polynucleotides encode for one or more fusion polypeptides. Each fusion polypeptide has two components: (i) an RNA binding protein or a fragment thereof, e.g., a repressor or a biologically active fragment thereof, fused to (ii) a destabilization domain.

### Repressor

In some embodiments, the repressor is chosen from the molecules provided in Table 1, e.g., Snu13, 50S ribosomal L7Ae protein, Pumilio and FBF (PUF) protein, PUF2 protein, MBP-LacZ, MBP, PCP, Lambda N, U1A, 15.5 kd, LARP7, L30e, or a variant or fragment thereof.

Snu13 is a nuclear protein that binds to the kink turn motif in the 5′ stem-loop of U4 snRNA and is a component of the [U4/U6.U5]tri-snRNP.

L7Ae is an archaeal ribosomal protein which regulates the translation of a designed mRNA in vitro and in human cells (see, e.g., Saito H, et al., Nat Chem Biol. 2010 January; 6(1):71-8; and Wroblewska L, et al. Nat Biotechnol. 2015; 33(8):839-841).

In some embodiments, when the repressor is 50S ribosomal L7Ae protein (e.g., wildtype 50S ribosomal L7Ae protein, a variant or fragment thereof) the binding element is a kink-turn forming sequence (e.g., SEQ ID NO:7, or a variant or fragment thereof).

PUF is a family of proteins that bind RNA sequence stretches defined by their amino acid identities at specific positions. Some amino acids in the protein can be engineered to change binding to any other RNA sequence. PUF2 is such an engineered protein. Filipovska A, et al. Nat Chem Biol. 2011 May 15; 7(7):425-7.

In some embodiments, when the repressor is PUF (e.g., wildtype PUF, or a variant or fragment thereof) the repressor binding element is PRE (e.g., wildtype PRE, or a variant or fragment thereof).

In some embodiments, when the repressor is PUF2 (e.g., wildtype PUF2, or a variant or fragment thereof) the repressor binding element is PRE2 (e.g., wildtype PRE2, or a variant or fragment thereof).

In some embodiments, when the repressor is MBP (e.g., wildtype MBP, a variant or fragment thereof) the repressor binding element is MS2 (e.g., wildtype MS2, or a variant or fragment thereof). In some instances, MBP predimerizes prior to binding to MS2 hairpins.

In some embodiments, when the repressor is MBP-LacZ or a variant or fragment thereof, the repressor binding element is MS2 (e.g., wildtype MS2, or a variant or fragment thereof).

In some embodiments, when the repressor is PCP (e.g., wildtype PCP, or a variant or fragment thereof) the repressor binding element is PP7 (e.g., wildtype PP7, or a variant or fragment thereof).

In some embodiments, when the repressor is Lambda N (e.g., wildtype Lambda N, or a variant or fragment thereof) the repressor binding element is BoxB (e.g., wildtype BoxB, or a variant or fragment thereof).

In some embodiments, when the repressor is U1A (e.g., wildtype U1A, or a variant or fragment thereof) the repressor binding element is U1A hairpin (e.g., wildtype U1A hairpin, or a variant or fragment thereof).

In some embodiments, when the repressor is 15.5 kd (e.g., wildtype 15.5 kd, or a variant or fragment thereof) the repressor binding element is a kink-turn forming sequence (e.g., wildtype U1A hairpin, or a variant or fragment thereof).

In some embodiments, when the repressor is LARP7 (e.g., wildtype LARP7, or a variant or fragment thereof) the repressor binding element is 7SK (e.g., wildtype 7SK, or a variant or fragment thereof).

In some embodiments, a repressor comprises an RNA-binding protein or a variant or a fragment thereof. Exemplary RNA-binding proteins are provided in Tables 2 and 3.

TABLE 1
Exemplary repressors and repressor binding elements

| RNA binding | RNA | Recognition |
|---|---|---|
| protein (RBP) | element | basis |
| PUF | PRE | sequence |
| PUF2 | PRE2 | sequence |
| MBP | MS2 | structure |
| MBP-LacZ | MS2 | structure |
| PCP | PP7 | structure |
| Lambda N | BoxB | structure |
| U1A | U1A hairpin | structure |
| 15.5 kd | Kink-turn | structure |
| | forming sequence | |
| 50S ribosomal | Kink-turn | structure |
| L7Ae protein | forming sequence | |
| LARP7 | 7SK | structure |
| Snu13 | Kink-turn | structure |
| | forming sequence | |

Additional exemplary RNA-binding proteins or RNA-binding domains which can be used as repressors are disclosed in Corley et al, Molecular Cell 781 pp. 9-29, the entire contents of which are hereby incorporated by reference. For example, Table 2 provides additional exemplary RNA-binding proteins or domains which can be used as repressors. In an embodiment, a repressor disclosed herein comprises a domain (or a variant, or a fragment thereof) or a protein (or a variant or a fragment thereof) listed in Table 2.

TABLE 2
Exemplary RNA-binding proteins and domains

| | Protein family |
|---|---|
| Domain name | containing domain |
| Cold shock domain | Cold shock proteins, Y-box proteins |
| Double stranded RNA | RNAses, ADARs, DICER |
| binding domain | |
| Helicase | DExH/D-box, Ski2-like, RIGI-like, |
| | NS3, UPF1-like RNA binding helicases |
| Intrinsically disordered | Most RBPs |
| regions (IDR) | |
| K homology | hnRNPs, translation regulation |
| | proteins, |
| La motif (LAM) | La proteins, La-related proteins |
| | (LARPs) |
| Piwi-Argonaute-Zwille | Argonaute proteins, Dicer |
| (PAZ) | |
| P-element Induced Wimpy | Argonaute proteins, |
| Testis (PIWI) | |
| Pentatricopeptide repeat (PPR) | RNA editing proteins |
| Pseudouridine synthase and | RNA modifying enzymes, metabolic |
| archaeosine | enzymes |
| transglycoslyase (PUA) | |
| Pumillo-like repeat (PUM) | PUF proteins |
| Ribosomal S1-like | Ribosomal proteins, Translation |
| | initiation factors, RNase II, PNPase |
| RNA recognition motif (RRM) | hnRNPs, splicing factors |
| Sm and Like-Sm (Sm/Lsm) | U1 spliceosomal proteins, Hfq |
| thiouridine synthases, RNA | tRNA modifying enzyme |
| methylases and pseudouridine | |
| synthases (THUMP) | |
| YT521-B homology (YTH) | YTH family m6A readers |
| Zn finger | Transcription factors, METTL |
| | enzymes, |

In some embodiments, the repressor comprises MBP. In some embodiments, the repressor comprises an amino acid sequence provided in Table 3 or a sequence with at least 80%, 85%, 90%, 95%, 96%, 97%, 98%, 99% or 100% identity thereof. In some embodiments, the repressor comprises the amino acid sequence of SEQ TD NO: 21, or an amino acid sequence with at least 80% 85%, 90%, 95%, 96%, 97%, 98%, 99% or 100% identity thereof.

In some embodiments, the repressor is encoded by a nucleotide sequence provided in Table 3 or a sequence with at least 80%, 85%, 90%, 95%, 96%, 97%, 98%, 99% or 100% identity thereof. In some embodiments, the repressor is encoded by the nucleotide sequence of SEQ ID NO: 22, or a sequence with at least 80%, 85%, 90%, 95%, 96%, 97%, 98%, 99% or 100% identity thereof.

TABLE 3
Exemplary sequences of repressor molecules

| | | |
|---|---|---|
| SEQ | Sequence | |
| ID NO | information | Sequence |

Repressor sequence

| | | |
|---|---|---|
| 21 | MBP (aa) | MASNFTQFVLVDNGGTGDVTVAPSNFANGIAEWISSNSRSQAYKVTCS |
| | | VRQSSAQNRKYTIKVEVPKGAWRSYLNMELTIPIFATNSDCELIVKAMQ |
| | | GLLKDGNPIPSAIAANSGIY |
| 22 | MBP (nt) | ATGGCCAGCAACTTCACCCAGTTCGTGCTGGTGGACAACGGCGGTA |
| | | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTAC |
| 25 | MBP- | MASNFTQFVLVDNGGTGDVTVAPSNFANGIAEWISSNSRSQAYKVTCS |
| | eIF4GdN | VRQSSAQNRKYTIKVEVPKGAWRSYLNMELTIPIFATNSDCELIVKAMQ |
| | (aa) | GLLKDGNPIPSAIAANSGIYGGGGSIFASMQKPEGLPHISDVVLDKANKT |
| | | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADK |
| | | DRGEEDADGSKTQDLFRRVRSILNKLTPQMFQQLMKQVTQLAIDTEER |
| | | LKGVIDLIFEKAISEPNFSVAYANMCRCLMALKVPTTEKPTVTVNFRKL |
| | | LLNRCQKEFEKDKDDDEVFEKKQKEMDEAATAEERGRLKEELEEARDI |
| | | ARRRSLGNIKFIGELFKLKMLTEAIMHDCVVKLLKNHDEESLECLCRLL |
| | | TTIGKDLDFEKAKPRMDQYFNQMEKIIKEKKTSSRIRFMLQDVLDLRGS |
| | | NWVPRRGDQGPKTIDQIHKEAEMEEHREHIKVQQLMAKGSDKRRGGP |
| | | PGPPISRGLPLVDDGGWNTVPISKGSRPIDTSRLTKITKPGSIDSNNQLFA |
| | | PGGRLSWGKGSSGGSGAKPSDAASEAARPATSTLNRFSALQQAVPTEST |
| | | DNRRVVQRSSLSRERGEKAGDRGDRLERSERGGDRGDRLDRARTPATK |
| | | RSFSKEVEERSRERPSQPEGLRKAASLTEDRDRGRDAVKREAALPPVSP |
| | | LKAALSEEELEKKSKAIIEEYLHLNDMKEAVQCVQELASPSLLFIFVRHG |
| | | VESTLERSAIAREHMGQLLHQLLCAGHLSTAQYYQGLYEILELAEDMEI |
| | | DIPHVWLYLAELVTPILQEGGVPMGELFREITKPLRPLGKAASLLLEILG |
| | | LLCKSMGPKKVGTLWREAGLSWKEFLPEGQDIGAFVAEQKVEYTLGEE |
| | | SEAPGQRALPSEELNRQLEKLLKEGSSNQRVFDWIEANLSEQQIVSNTL |
| | | VRALMTAVCYSAIIFETPLRVDVAVLKARAKLLQKYLCDEQKELQALY |
| | | ALQALVVTLEQPPNLLRMFFDALYDEDVVKEDAFYSWESSKDPAEQQ |
| | | GKGVALKSVTAFFKWLREAEEESDHN |
| 26 | MBP- | ATGGCCAGCAACTTCACCCAGTTCGTGCTGGTGGACAACGGCGGTA |
| | eIF4GdN (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCATA |
| | | TTCGCGAGCATGCAGAAGCCTGAAGGTCTGCCTCACATCAGCGACG |
| | | TGGTGCTGGACAAGGCCAACAAGACCCCTCTTAGACCTCTGGACCCT |
| | | ACCAGACTGCAGGGCATCAACTGCGGCCCTGACTTCACTCCTTCTTT |
| | | CGCAAACCTGGGCAGAACCACCCTGAGCACCAGAGGCCCTCCTAGA |
| | | GGTGGTCCCGGCGGAGAACTCCCCAGGGGTCCTGCCGGCCTGGGCC |
| | | CTAGACGCTCCCAGCAAGGTCCTAGAAAGGAGCCAAGGAAGATCAT |
| | | CGCCACCGTGCTGATGACCGAGGACATCAAGCTGAACAAAGCTGAG |
| | | AAGGCCTGGAAGCCTAGCAGCAAGAGAACCGCCGCCGACAAGGAC |
| | | AGAGGCGAGGAGGACGCCGACGGATCCAAGACCCAGGACCTGTTCA |
| | | GAAGAGTGAGAAGCATCCTCAACAAGCTGACCCCTCAGATGTTCCA |
| | | GCAGCTGATGAAGCAGGTGACGCAGCTCGCCATCGACACCGAGGAG |
| | | AGACTGAAGGGCGTGATCGACCTGATCTTTGAGAAGGCTATCTCAG |
| | | AGCCTAACTTCAGCGTGGCCTACGCCAACATGTGCCGTTGCCTGATG |
| | | GCATTGAAGGTGCCAACCACCGAGAAGCCTACTGTGACCGTCAATTT |
| | | CCGTAAACTGCTGCTGAACCGGTGCCAGAAAGAGTTCGAGAAGGAT |
| | | AAGGACGACGACGAGGTCTTCGAGAAGAAACAGAAAGAAATGGAC |
| | | GAGGCCGCCACCGCAGAGGAAAGGGGCCGATTAAAGGAGGAGCTG |
| | | GAGGAGGCCAGAGACATCGCCAGACGGCGTTCTCTGGGCAACATCA |
| | | AGTTCATAGGTGAGCTGTTCAAGCTAAAGATGCTCACCGAGGCCAT |
| | | AATGCACGACTGCGTGGTGAAGCTACTGAAGAACCACGACGAAGAA |
| | | AGCCTGGAGTGCCTGTGCAGACTGCTGACCACCATCGGCAAGGACC |
| | | TGGACTTCGAGAAGGCAAAGCCTCGAATGGACCAGTACTTCAACCA |
| | | GATGGAGAAGATTATCAAGGAGAAGAAGACCAGCAGCAGAATCAG |
| | | ATTCATGCTGCAGGACGTACTGGACCTGCGCGGAAGCAACTGGGTG |
| | | CCAAGGAGAGGGGACCAAGGACCAAAGACCATCGACCAGATCCAC |
| | | AAGGAAGCGGAGATGGAGGAGCACAGAGAGCACATAAAGGTGCAG |
| | | CAGCTTATGGCCAAGGGCAGCGACAAGCGAAGAGGCGGCCCGCCCG |
| | | GACCTCCTATCAGCAGAGGCCTTCCTCTGGTAGACGACGGCGGCTGG |
| | | AACACCGTGCCTATCTCTAAGGGCTCCAGACCTATCGACACTTCCCG |
| | | TCTTACCAAGATCACCAAGCCAGGATCTATTGACAGCAACAACCAG |
| | | CTGTTCGCCCCAGGAGGAAGACTTAGCTGGGGCAAGGGAAGTTCCG |
| | | GCGGATCCGGCGCCAAGCCTTCCGACGCCGCCAGCGAGGCTGCCAG |
| | | ACCTGCCACCAGCACCTTGAACCGCTTTTCCGCTCTGCAGCAAGCTG |
| | | TGCCGACCGAGAGCACCGACAACCGGCGAGTTGTGCAGAGAAGCAG |
| | | CCTGAGCAGAGAGAGGGGCGAGAAAGCCGGCGATAGAGGTGACCG |
| | | ACTGGAGAGAAGCGAGAGAGGAGGTGATAGAGGCGACCGGCTTGA |
| | | CAGAGCCAGAACCCCTGCCACAAAGCGATCGTTCAGCAAGGAGGTC |
| | | GAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 27 | MB_eIF4G | MASNFTQFVLVDNGGTGDVTVAPSNFANGIAEWISSNSRSQAYKVTCS |
| | (fl) (aa) | VRQSSAQNRKYTIKVEVPKGAWRSYLNMELTIPIFATNSDCELIVKAMQ |
| | | GLLKDGNPIPSAIAANSGIYGGGGSNKAPQSTGPPPAPSPGLPQPAFPPG |
| | | QTAPVVFSTPQATQMNTPSQPRQHFYPSRAQPPSSAASRVQSAAPARPG |
| | | PAAHVYPAGSQVMMIPSQISYPASQGAYYIPGQGRSTYVVPTQQYPVQP |
| | | GAPGFYPGASPTEFGTYAGAYYPAQGVQQFPTGVAPTPVLMNQPPQIA |
| | | PKRERKTIRIRDPNQGGKDITEEIMSGARTASTPTPPQTGGGLEPQANGE |
| | | TPQVAVIVRPDDRSQGAIIADRPGLPGPEHSPSESQPSSPSPTPSPSPVLEP |
| | | GSEPNLAVLSIPGDTMTTIQMSVEESTPISRETGEPYRLSPEPTPLAEPILE |
| | | VEVTLSKPVPESEFSSSPLQAPTPLASHTVEIHEPNGMVPSEDLEPEVESS |
| | | PELAPPPACPSESPVPIAPTAQPEELLNGAPSPPAVDLSPVSEPEEQAKEV |
| | | TASMAPPTIPSATPATAPSATSPAQEEEMEEEEEEEEGEAGEAGEAESEK |
| | | GGEELLPPESTPIPANLSQNLEAAAATQVAVSVPKRRRKIKELNKKEAV |
| | | GDLLDAFKEANPAVPEVENQPPAGSNPGPESEGSGVPPRPEEADETWDS |
| | | KEDKIHNAENIQPGEQKYEYKSDQWKPLNLEEKKRYDREFLLGFQFIFA |
| | | SMQKPEGLPHISDVVLDKANKTPLRPLDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMH |
| | | DCVVKLLKNHDEESLECLCRLLTTIGKDLDFEKAKPRMDQYFNQMEKII |
| | | KEKKTSSRIRFMLQDVLDLRGSNWVPRRGDQGPKTIDQIHKEAEMEEH |
| | | REHIKVQQLMAKGSDKRRGGPPGPPISRGLPLVDDGGWNTVPISKGSRP |
| | | IDTSRLTKITKPGSIDSNNQLFAPGGRLSWGKGSSGGSGAKPSDAASEAA |
| | | RPATSTLNRFSALQQAVPTESTDNRRVVQRSSLSRERGEKAGDRGDRLE |
| | | RSERGGDRGDRLDRARTPATKRSFSKEVEERSRERPSQPEGLRKAASLT |
| | | EDRDRGRDAVKREAALPPVSPLKAALSEEELEKKSKAIIEEYLHLNDMK |
| | | EAVQCVQELASPSLLFIFVRHGVESTLERSAIAREHMGQLLHQLLCAGH |
| | | LSTAQYYQGLYEILELAEDMEIDIPHVWLYLAELVTPILQEGGVPMGEL |
| | | FREITKPLRPLGKAASLLLEILGLLCKSMGPKKVGTLWREAGLSWKEFL |
| | | PEGQDIGAFVAEQKVEYTLGEESEAPGQRALPSEELNRQLEKLLKEGSS |
| | | NQRVFDWIEANLSEQQIVSNTLVRALMTAVCYSAIIFETPLRVDVAVLK |
| | | ARAKLLQKYLCDEQKELQALYALQALVVTLEQPPNLLRMFFDALYDE |
| | | DVVKEDAFYSWESSKDPAEQQGKGVALKSVTAFFKWLREAEEESDHN |
| 28 | MBP_eIF4G | ATGGCCAGCAACTTCACCCAGTTCGTGCTGGTGGACAACGGCGGTA |
| | (fl) (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAAC |
| | | AAGGCCCCTCAGAGCACCGGCCCTCCTCCTGCCCCTAGCCCTGGCCT |
| | | GCCTCAGCCTGCCTTCCCTCCTGGCCAGACCGCCCCTGTGGTGTTCA |
| | | GCACCCCTCAGGCCACCCAGATGAACACACCTAGCCAGCCTAGACA |
| | | GCACTTCTACCCTAGCAGAGCCCAGCCTCCTAGCAGCGCCGCCAGCA |
| | | GAGTGCAGTCCGCTGCACCTGCCAGACCTGGCCCTGCCGCCCACGTG |
| | | TACCCTGCCGGCAGCCAGGTGATGATGATCCCGAGTCAAATCAGCT |
| | | ACCCTGCAAGCCAGGGCGCCTACTACATCCCGGGCCAGGGCAGAAG |
| | | CACCTACGTGGTGCCTACCCAGCAGTACCCTGTGCAGCCTGGCGCCC |
| | | CTGGTTTCTATCCTGGCGCAAGCCCTACCGAGTTCGGAACTTACGCC |
| | | GGCGCTTACTATCCAGCTCAGGGCGTGCAGCAGTTCCCTACCGGCGT |
| | | GGCCCCTACCCCTGTGCTGATGAATCAGCCACCTCAGATCGCCCCTA |
| | | AGCGCGAGCGCAAGACCATCAGAATCCGCGATCCTAACCAGGGCGG |
| | | CAAGGACATCACCGAGGAGATCATGAGCGGCGCCAGAACAGCAAGT |
| | | ACTCCAACCCCGCCACAAACCGGCGGCGGCCTGGAGCCTCAAGCCA |
| | | ACGGCGAGACGCCACAAGTGGCCGTGATCGTACGCCCTGACGACCG |
| | | GAGCCAAGGTGCAATCATCGCCGATAGGCCTGGCCTCCCAGGTCCG |
| | | GAGCACAGCCCTAGCGAGTCCCAGCCGTCTTCACCATCACCAACCCC |
| | | TAGTCCATCCCCTGTTCTCGAACCAGGCAGCGAGCCTAACCTGGCCG |
| | | TGCTGAGCATACCAGGTGACACCATGACCACCATCCAGATGAGCGT |
| | | GGAGGAGAGCACCCCAATCAGCAGAGAAACTGGAGAGCCTTACAG |
| | | ACTGTCCCCAGAGCCGACCCCACTGGCCGAGCCAATACTGGAGGTG |
| | | GAGGTGACCCTGAGCAAGCCTGTGCCTGAGAGCGAGTTCAGCTCCT |
| | | CTCCACTGCAGGCCCCAACTCCTCTCGCAAGCCACACCGTGGAGATC |
| | | CACGAACCTAACGGCATGGTACCAAGCGAAGATCTTGAGCCAGAGG |
| | | TCGAATCAAGCCCAGAACTGGCCCCTCCACCTGCCTGCCCGTCTGAA |
| | | TCTCCGGTCCCTATCGCTCCTACGGCACAGCCTGAGGAGCTGCTGAA |
| | | CGGTGCCCCGAGCCCTCCAGCAGTGGACTTATCCCCAGTATCAGAGC |
| | | CTGAAGAACAGGCCAAGGAGGTAACTGCCTCTATGGCGCCACCTAC |
| | | CATACCTTCGGCAACACCGGCTACAGCACCATCTGCGACTAGTCCGG |
| | | CTCAGGAGGAGGAGATGGAGGAAGAAGAAGAGGAAGAGGAGGGCG |
| | | AGGCCGGAGAGGCCGGTGAAGCCGAGTCCGAGAAGGGCGGCGAAG |
| | | AACTTCTCCCTCCAGAGTCAACTCCTATCCCTGCCAACCTTAGTCAG |
| | | AATCTGGAGGCCGCCGCCGCTACTCAGGTTGCAGTGAGCGTGCCAA |
| | | AGAGACGTCGCAAGATCAAGGAGCTGAACAAGAAGGAGGCCGTGG |
| | | GCGACCTGCTGGACGCCTTCAAGGAGGCAAACCCGGCGGTGCCTGA |
| | | AGTGGAGAATCAGCCTCCGGCCGGATCAAACCCTGGTCCTGAGAGT |
| | | GAAGGCAGCGGCGTCCCACCAAGACCTGAAGAGGCTGACGAAACTT |
| | | GGGACAGCAAGGAGGACAAGATCCACAACGCCGAGAACATCCAGC |
| | | CAGGCGAGCAGAAGTACGAGTACAAGAGCGACCAGTGGAAGCCTCT |
| | | TAACCTTGAAGAGAAGAAGAGATACGACAGAGAGTTCCTGCTGGGC |
| | | TTCCAGTTCATATTCGCGAGCATGCAGAAGCCTGAAGGTCTGCCTCA |
| | | CATCAGCGACGTGGTGCTGGACAAGGCCAACAAGACCCCTCTTAGA |
| | | CCTCTGGACCCTACCAGACTGCAGGGCATCAACTGCGGCCCTGACTT |
| | | CACTCCTTCTTTCGCAAACCTGGGCAGAACCACCCTGAGCACCAGAG |
| | | GCCCTCCTAGAGGTGGTCCCGGCGGAGAACTCCCCAGGGGTCCTGC |
| | | CGGCCTGGGCCCTAGACGCTCCCAGCAAGGTCCTAGAAAGGAGCCA |
| | | AGGAAGATCATCGCCACCGTGCTGATGACCGAGGACATCAAGCTGA |
| | | ACAAAGCTGAGAAGGCCTGGAAGCCTAGCAGCAAGAGAACCGCCG |
| | | CCGACAAGGACAGAGGCGAGGAGGACGCCGACGGATCCAAGACCC |
| | | AGGACCTGTTCAGAAGAGTGAGAAGCATCCTCAACAAGCTGACCCC |
| | | TCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAG |
| | | TTCGAGAAGGATAAGGACGACGACGAGGTCTTCGAGAAGAAACAG |
| | | AAAGAAATGGACGAGGCCGCCACCGCAGAGGAAAGGGGCCGATTA |
| | | AAGGAGGAGCTGGAGGAGGCCAGAGACATCGCCAGACGGCGTTCTC |
| | | TGGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTGCCGACCGAGAGCACCGACAACCGGCGAGTTGTGC |
| | | AGAGAAGCAGCCTGAGCAGAGAGAGGGGCGAGAAAGCCGGCGATA |
| | | GAGGTGACCGACTGGAGAGAAGCGAGAGAGGAGGTGATAGAGGCG |
| | | ACCGGCTTGACAGAGCCAGAACCCCTGCCACAAAGCGATCGTTCAG |
| | | CAAGGAGGTCGAAGAGAGGTCCAGGGAGCGCCCTAGCCAGCCGGA |
| | | AGGACTCAGAAAGGCAGCCAGTCTAACAGAGGACCGCGACAGGGG |
| | | AAGAGACGCCGTGAAGAGGGAGGCCGCACTGCCTCCTGTGAGCCCT |
| | | CTGAAGGCGGCACTGAGCGAAGAAGAACTTGAGAAGAAGAGTAAG |
| | | GCGATCATCGAGGAGTACCTGCACCTGAACGACATGAAGGAGGCCG |
| | | TGCAGTGCGTGCAAGAGCTCGCGAGCCCATCACTGCTGTTCATCTTC |
| | | GTCCGGCACGGCGTGGAGTCCACACTGGAAAGATCTGCCATTGCTA |
| | | GGGAGCATATGGGCCAGTTGTTGCACCAATTGCTTTGCGCCGGCCAC |
| | | CTAAGTACTGCCCAGTACTATCAGGGTTTATACGAGATCCTCGAACT |
| | | GGCCGAAGACATGGAGATCGACATCCCTCACGTGTGGCTGTACCTC |
| | | GCAGAACTGGTGACCCCTATCCTGCAGGAGGGCGGCGTTCCAATGG |
| | | GTGAGTTGTTCAGAGAAATCACAAAGCCACTGCGCCCACTGGGCAA |
| | | GGCGGCTAGTCTCCTTCTGGAGATTCTCGGCCTGCTCTGTAAGAGCA |
| | | TGGGTCCAAAGAAGGTGGGCACCCTGTGGAGGGAAGCTGGACTCTC |
| | | CTGGAAGGAATTCCTCCCTGAGGGTCAGGACATCGGCGCCTTCGTGG |
| | | CCGAACAGAAGGTTGAGTACACCCTGGGAGAGGAATCGGAAGCGCC |
| | | GGGTCAGCGGGCTCTGCCGAGTGAGGAGCTCAACAGACAACTCGAG |
| | | AAGCTCCTCAAGGAGGGATCCTCAAATCAGAGAGTGTTCGACTGGA |
| | | TCGAGGCCAATCTCAGCGAGCAGCAAATCGTGAGCAACACGTTGGT |
| | | TCGCGCTCTCATGACAGCCGTGTGCTACTCAGCCATTATCTTCGAGA |
| | | CGCCTCTCCGCGTGGACGTGGCAGTGCTCAAGGCCCGCGCTAAGCTG |
| | | TTACAGAAGTACCTGTGCGACGAGCAGAAGGAACTGCAGGCCCTGT |
| | | ACGCCCTGCAAGCTCTGGTCGTCACACTCGAGCAGCCTCCTAACCTG |
| | | CTGAGAATGTTCTTCGACGCATTGTACGACGAGGACGTTGTGAAGG |
| | | AAGACGCTTTCTACAGCTGGGAGTCTAGTAAGGATCCTGCGGAACA |
| | | ACAAGGCAAGGGCGTTGCTCTAAAGAGCGTGACCGCCTTCTTCAAG |
| | | TGGCTGCGAGAGGCTGAGGAAGAGTCGGACCACAAC |
| 29 | MBP_eIF4G- | MASNFTQFVLVDNGGTGDVTVAPSNFANGIAEWISSNSRSQAYKVTCS |
| | dN (623- | VRQSSAQNRKYTIKVEVPKGAWRSYLNMELTIPIFATNSDCELIVKAMQ |
| | 1599, 3A) | GLLKDGNPIPSAIAANSGIYGGGGSIFASMQKPEGLPHISDVVLDKANKT |
| | (aa) | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADK |
| | | DRGEEDADGSKTQDLFRRVRSIANKATPQMAQQLMKQVTQLAIDTEER |
| | | LKGVIDLIFEKAISEPNFSVAYANMCRCLMALKVPTTEKPTVTVNFRKL |
| | | LLNRCQKEFEKDKDDDEVFEKKQKEMDEAATAEERGRLKEELEEARDI |
| | | ARRRSLGNIKFIGELFKLKMLTEAIMHDCVVKLLKNHDEESLECLCRLL |
| | | TTIGKDLDFEKAKPRMDQYFNQMEKIIKEKKTSSRIRFMLQDVLDLRGS |
| | | NWVPRRGDQGPKTIDQIHKEAEMEEHREHIKVQQLMAKGSDKRRGGP |
| | | PGPPISRGLPLVDDGGWNTVPISKGSRPIDTSRLTKITKPGSIDSNNQLFA |
| | | PGGRLSWGKGSSGG |
| | | SGAKPSDAASEAARPATSTLNRFSALQQAVPTESTDNRRVVQRSSLSRE |
| | | RGEKAGDRGDRLERSERGGDRGDRLDRARTPATKRSFSKEVEERSRER |
| | | PSQPEGLRKAASLTEDRDRGRDAVKREAALPPVSPLKAALSEEELEKKS |
| | | KAIIEEYLHLNDMKEAVQCVQELASPSLLFIFVRHGVESTLERSAIAREH |
| | | MGQLLHQLLCAGHLSTAQYYQGLYEILELAEDMEIDIPHVWLYLAELV |
| | | TPILQEGGVPMGELFREITKPLRPLGKAASLLLEILGLLCKSMGPKKVGT |
| | | LWREAGLSWKEFLPEGQDIGAFVAEQKVEYTLGEESEAPGQRALPSEEL |
| | | NRQLEKLLKEGSSNQRVFDWIEANLSEQQIVSNTLVRALMTAVCYSAII |
| | | FETPLRVDVAVLKARAKLLQKYLCDEQKELQALYALQALVVTLEQPPN |
| | | LLRMFFDALYDEDVVKEDAFYSWESSKDPAEQQGKGVALK |
| | | SVTAFFKWLREAEEESDHN |
| 30 | MBP_eIF4G- | ATGGCCAGCAACTTCACCCAGTTCGTGCTGGTGGACAACGGCGGTA |
| | dN (623- | CCGGAGACGTGACCGTGGCCCCTTCTAACTTCGCCAACGGCATCGCC |
| | 1599, 3A) | GAGTGGATCAGCAGCAACAGCAGAAGCCAGGCCTACAAGGTGACCT |
| | (nt) | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCATA |
| | | TTCGCGAGCATGCAGAAGCCTGAAGGTCTGCCTCACATCAGCGACG |
| | | TGGTGCTGGACAAGGCCAACAAGACCCCTCTTAGACCTCTGGACCCT |
| | | ACCAGACTGCAGGGCATCAACTGCGGCCCTGACTTCACTCCTTCTTT |
| | | CGCAAACCTGGGCAGAACCACCCTGAGCACCAGAGGCCCTCCTAGA |
| | | GGTGGTCCCGGCGGAGAACTCCCCAGGGGTCCTGCCGGCCTGGGCC |
| | | CTAGACGCTCCCAGCAAGGTCCTAGAAAGGAGCCAAGGAAGATCAT |
| | | CGCCACCGTGCTGATGACCGAGGACATCAAGCTGAACAAAGCTGAG |
| | | AAGGCCTGGAAGCCTAGCAGCAAGAGAACCGCCGCCGACAAGGAC |
| | | AGAGGCGAGGAGGACGCCGACGGATCCAAGACCCAGGACCTGTTCA |
| | | GAAGAGTGAGAAGCATCGCGAACAAGGCGACCCCTCAGATGGCGCA |
| | | GCAGCTGATGAAGCAGGTGACGCAGCTCGCCATCGACACCGAGGAG |
| | | AGACTGAAGGGCGTGATCGACCTGATCTTTGAGAAGGCTATCTCAG |
| | | AGCCTAACTTCAGCGTGGCCTACGCCAACATGTGCCGTTGCCTGATG |
| | | GCATTGAAGGTGCCAACCACCGAGAAGCCTACTGTGACCGTCAATTT |
| | | CCGTAAACTGCTGCTGAACCGGTGCCAGAAAGAGTTCGAGAAGGAT |
| | | AAGGACGACGACGAGGTCTTCGAGAAGAAACAGAAAGAAATGGAC |
| | | GAGGCCGCCACCGCAGAGGAAAGGGGCCGATTAAAGGAGGAGCTG |
| | | GAGGAGGCCAGAGACATCGCCAGACGGCGTTCTCTGGGCAACATCA |
| | | AGTTCATAGGTGAGCTGTTCAAGCTAAAGATGCTCACCGAGGCCAT |
| | | AATGCACGACTGCGTGGTGAAGCTACTGAAGAACCACGACGAAGAA |
| | | AGCCTGGAGTGCCTGTGCAGACTGCTGACCACCATCGGCAAGGACC |
| | | TGGACTTCGAGAAGGCAAAGCCTCGAATGGACCAGTACTTCAACCA |
| | | GATGGAGAAGATTATCAAGGAGAAGAAGACCAGCAGCAGAATCAG |
| | | ATTCATGCTGCAGGACGTACTGGACCTGCGCGGAAGCAACTGGGTG |
| | | CCAAGGAGAGGGGACCAAGGACCAAAGACCATCGACCAGATCCAC |
| | | AAGGAAGCGGAGATGGAGGAGCACAGAGAGCACATAAAGGTGCAG |
| | | CAGCTTATGGCCAAGGGCAGCGACAAGCGAAGAGGCGGCCCGCCCG |
| | | GACCTCCTATCAGCAGAGGCCTTCCTCTGGTAGACGACGGCGGCTGG |
| | | AACACCGTGCCTATCTCTAAGGGCTCCAGACCTATCGACACTTCCCG |
| | | TCTTACCAAGATCACCAAGCCAGGATCTATTGACAGCAACAACCAG |
| | | CTGTTCGCCCCAGGAGGAAGACTTAGCTGGGGCAAGGGAAGTTCCG |
| | | GCGGATCCGGCGCCAAGCCTTCCGACGCCGCCAGCGAGGCTGCCAG |
| | | ACCTGCCACCAGCACCTTGAACCGCTTTTCCGCTCTGCAGCAAGCTG |
| | | TGCCGACCGAGAGCACCGACAACCGGCGAGTTGTGCAGAGAAGCAG |
| | | CCTGAGCAGAGAGAGGGGCGAGAAAGCCGGCGATAGAGGTGACCG |
| | | ACTGGAGAGAAGCGAGAGAGGAGGTGATAGAGGCGACCGGCTTGA |
| | | CAGAGCCAGAACCCCTGCCACAAAGCGATCGTTCAGCAAGGAGGTC |
| | | GAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 31 | MBP_eIF4G- | MASNFTQFVLVDNGGTGDVTVAPSNFANGIAEWISSNSRSQAYKVTCS |
| | dN2 (654- | VRQSSAQNRKYTIKVEVPKGAWRSYLNMELTIPIFATNSDCELIVKAMQ |
| | 1599) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAVPTESTDNRRVVQRSSLSRERGEKAGDRGDRLER |
| | | SERGGDRGDRLDRARTPATKRSFSKEVEERSRERPSQPEGLRKAASLTE |
| | | DRDRGRDAVKREAALPPVSPLKAALSEEELEKKSKAIIEEYLHLNDMKE |
| | | AVQCVQELASPSLLFIFVRHGVESTLERSAIAREHMGQLLHQLLCAGHL |
| | | STAQYYQGLYEILELAEDMEIDIPHVWLYLAELVTPILQEGGVPMGELF |
| | | REITKPLRPLGKAASLLLEILGLLCKSMGPKKVGTLWREAGLSWKEFLP |
| | | EGQDIGAFVAEQKVEYTLGEESEAPGQRALPSEELNRQLEKLLKEGSSN |
| | | QRVFDWIEANLSEQQIVSNTLVRALMTAVCYSAIIFETPLRVDVAVLKA |
| | | RAKLLQKYLCDEQKELQALYALQALVVTLEQPPNLLRMFFDALYDED |
| | | VVKEDAFYSWESSKDPAEQQGKGVALKSVTAFFKWLREAEEESDHN |
| 32 | MBP_eIF4G- | ATGGCCAGCAACTTCACCCAGTTCGTGCTGGTGGACAACGGCGGTA |
| | dN2 (654- | CCGGAGACGTGACCGTGGCCCCTTCTAACTTCGCCAACGGCATCGCC |
| | 1599) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| | | AAGGAGGGATCCTCAAATCAGAGAGTGTTCGACTGGATCGAGGCCA |
| | | ATCTCAGCGAGCAGCAAATCGTGAGCAACACGTTGGTTCGCGCTCTC |
| | | ATGACAGCCGTGTGCTACTCAGCCATTATCTTCGAGACGCCTCTCCG |
| | | CGTGGACGTGGCAGTGCTCAAGGCCCGCGCTAAGCTGTTACAGAAG |
| | | TACCTGTGCGACGAGCAGAAGGAACTGCAGGCCCTGTACGCCCTGC |
| | | AAGCTCTGGTCGTCACACTCGAGCAGCCTCCTAACCTGCTGAGAATG |
| | | TTCTTCGACGCATTGTACGACGAGGACGTTGTGAAGGAAGACGCTTT |
| | | CTACAGCTGGGAGTCTAGTAAGGATCCTGCGGAACAACAAGGCAAG |
| | | GGCGTTGCTCTAAAGAGCGTGACCGCCTTCTTCAAGTGGCTGCGAGA |
| | | GGCTGAGGAAGAGTCGGACCACAAC |
| 33 | MBP_eIF4G- | MASNFTQFVLVDNGGTGDVTVAPSNFANGIAEWISSNSRSQAYKVTCS |
| | dN3 (654- | VRQSSAQNRKYTIKVEVPKGAWRSYLNMELTIPIFATNSDCELIVKAMQ |
| | 1451) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAVPTESTDNRRVVQRSSLSRERGEKAGDRGDRLER |
| | | SERGGDRGDRLDRARTPATKRSFSKEVEERSRERPSQPEGLRKAASLTE |
| | | DRDRGRDAVKREAALPPVSPLKAALSEEELEKKSKAIIEEYLHLNDMKE |
| | | AVQCVQELASPSLLFIFVRHGVESTLERSAIAREHMGQLLHQLLCAGHL |
| | | STAQYYQGLYEILELAEDMEIDIPHVWLYLAELVTPILQEGGVPMGELF |
| | | REITKPLRPLGKAASLLLEILGLLCKSMGPKKVGTLWREAGLSWKEFLP |
| | | EGQDIGAFVAEQKVEYTLGEESEAPGQRALPSEELNRQLEKLL |
| 34 | MBP_eIF4G- | ATGGCCAGCAACTTCACCCAGTTCGTGCTGGTGGACAACGGCGGTA |
| | dN3 (654- | CCGGAGACGTGACCGTGGCCCCTTCTAACTTCGCCAACGGCATCGCC |
| | 1451) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| 35 | MBP_eIF4G- | MASNFTQFVLVDNGGTGDVTVAPSNFANGIAEWISSNSRSQAYKVTCS |
| | mid1 (674- | VRQSSAQNRKYTIKVEVPKGAWRSYLNMELTIPIFATNSDCELIVKAMQ |
| | 1079) (aa) | GLLKDGNPIPSAIAANSGIYGGGGSGRTTLSTRGPPRGGPGGELPRGPAG |
| | | LGPRRSQQGPRKEPRKIIATVLMTEDIKLNKAEKAWKPSSKRTAADKDR |
| | | GEEDADGSKTQDLFRRVRSILNKLTPQMFQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKFIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSID |
| 36 | MBP_eIF4G- | ATGGCCAGCAACTTCACCCAGTTCGTGCTGGTGGACAACGGCGGTA |
| | mid1 (674- | CCGGAGACGTGACCGTGGCCCCTTCTAACTTCGCCAACGGCATCGCC |
| | 1079) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGGC |
| | | AGAACCACCCTGAGCACCAGAGGCCCTCCTAGAGGTGGTCCCGGCG |
| | | GAGAACTCCCCAGGGGTCCTGCCGGCCTGGGCCCTAGACGCTCCCA |
| | | GCAAGGTCCTAGAAAGGAGCCAAGGAAGATCATCGCCACCGTGCTG |
| | | ATGACCGAGGACATCAAGCTGAACAAAGCTGAGAAGGCCTGGAAGC |
| | | CTAGCAGCAAGAGAACCGCCGCCGACAAGGACAGAGGCGAGGAGG |
| | | ACGCCGACGGATCCAAGACCCAGGACCTGTTCAGAAGAGTGAGAAG |
| | | CATCCTCAACAAGCTGACCCCTCAGATGTTCCAGCAGCTGATGAAGC |
| | | AGGTGACGCAGCTCGCCATCGACACCGAGGAGAGACTGAAGGGCGT |
| | | GATCGACCTGATCTTTGAGAAGGCTATCTCAGAGCCTAACTTCAGCG |
| | | TGGCCTACGCCAACATGTGCCGTTGCCTGATGGCATTGAAGGTGCCA |
| | | ACCACCGAGAAGCCTACTGTGACCGTCAATTTCCGTAAACTGCTGCT |
| | | GAACCGGTGCCAGAAAGAGTTCGAGAAGGATAAGGACGACGACGA |
| | | GGTCTTCGAGAAGAAACAGAAAGAAATGGACGAGGCCGCCACCGC |
| | | AGAGGAAAGGGGCCGATTAAAGGAGGAGCTGGAGGAGGCCAGAGA |
| | | CATCGCCAGACGGCGTTCTCTGGGCAACATCAAGTTCATAGGTGAGC |
| | | TGTTCAAGCTAAAGATGCTCACCGAGGCCATAATGCACGACTGCGT |
| | | GGTGAAGCTACTGAAGAACCACGACGAAGAAAGCCTGGAGTGCCTG |
| | | TGCAGACTGCTGACCACCATCGGCAAGGACCTGGACTTCGAGAAGG |
| | | CAAAGCCTCGAATGGACCAGTACTTCAACCAGATGGAGAAGATTAT |
| | | CAAGGAGAAGAAGACCAGCAGCAGAATCAGATTCATGCTGCAGGAC |
| | | GTACTGGACCTGCGCGGAAGCAACTGGGTGCCAAGGAGAGGGGACC |
| | | AAGGACCAAAGACCATCGACCAGATCCACAAGGAAGCGGAGATGG |
| | | AGGAGCACAGAGAGCACATAAAGGTGCAGCAGCTTATGGCCAAGG |
| | | GCAGCGACAAGCGAAGAGGCGGCCCGCCCGGACCTCCTATCAGCAG |
| | | AGGCCTTCCTCTGGTAGACGACGGCGGCTGGAACACCGTGCCTATCT |
| | | CTAAGGGCTCCAGACCTATCGACACTTCCCGTCTTACCAAGATCACC |
| | | AAGCCAGGATCTATTGAC |
| 37 | MBP_eIF4G- | MASNFTQFVLVDNGGTGDVTVAPSNFANGIAEWISSNSRSQAYKVTCS |
| | mid2 (654- | VRQSSAQNRKYTIKVEVPKGAWRSYLNMELTIPIFATNSDCELIVKAMQ |
| | 1130) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAV |
| 38 | MBP_eIF4G- | ATGGCCAGCAACTTCACCCAGTTCGTGCTGGTGGACAACGGCGGTA |
| | mid2 (654- | CCGGAGACGTGACCGTGGCCCCTTCTAACTTCGCCAACGGCATCGCC |
| | 1130) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTG |
| 39 | MBP_eIF4G- | MASNFTQFVLVDNGGTGDVTVAPSNFANGIAEWISSNSRSQAYKVTCS |
| | mid3 (712- | VRQSSAQNRKYTIKVEVPKGAWRSYLNMELTIPIFATNSDCELIVKAMQ |
| | 1130) (aa) | GLLKDGNPIPSAIAANSGIYGGGGSPRKIIATVLMTEDIKLNKAEKAWKP |
| | | SSKRTAADKDRGEEDADGSKTQDLFRRVRSILNKLTPQMFQQLMKQVT |
| | | QLAIDTEERLKGVIDLIFEKAISEPNFSVAYANMCRCLMALKVPTTEKPT |
| | | VTVNFRKLLLNRCQKEFEKDKDDDEVFEKKQKEMDEAATAEERGRLK |
| | | EELEEARDIARRRSLGNIKFIGELFKLKMLTEAIMHDCVVKLLKNHDEE |
| | | SLECLCRLLTTIGKDLDFEKAKPRMDQYFNQMEKIIKEKKTSSRIRFMLQ |
| | | DVLDLRGSNWVPRRGDQGPKTIDQIHKEAEMEEHREHIKVQQLMAKG |
| | | SDKRRGGPPGPPISRGLPLVDDGGWNTVPISKGSRPIDTSRLTKITKPGSI |
| | | DSNNQLFAPGGRLSWGKGSSGGSGAKPSDAASEAARPATSTLNRFSAL |
| | | QQAV |
| 40 | MBP_eIF4G- | ATGGCCAGCAACTTCACCCAGTTCGTGCTGGTGGACAACGGCGGTA |
| | mid3 (712- | CCGGAGACGTGACCGTGGCCCCTTCTAACTTCGCCAACGGCATCGCC |
| | 1130) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCCCA |
| | | AGGAAGATCATCGCCACCGTGCTGATGACCGAGGACATCAAGCTGA |
| | | ACAAAGCTGAGAAGGCCTGGAAGCCTAGCAGCAAGAGAACCGCCG |
| | | CCGACAAGGACAGAGGCGAGGAGGACGCCGACGGATCCAAGACCC |
| | | AGGACCTGTTCAGAAGAGTGAGAAGCATCCTCAACAAGCTGACCCC |
| | | TCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAG |
| | | TTCGAGAAGGATAAGGACGACGACGAGGTCTTCGAGAAGAAACAG |
| | | AAAGAAATGGACGAGGCCGCCACCGCAGAGGAAAGGGGCCGATTA |
| | | AAGGAGGAGCTGGAGGAGGCCAGAGACATCGCCAGACGGCGTTCTC |
| | | TGGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTG |
| 41 | MBP_eIF4G- | MASNFTQFVLVDNGGTGDVTVAPSNFANGIAEWISSNSRSQAYKVTCS |
| | C1 (1080- | VRQSSAQNRKYTIKVEVPKGAWRSYLNMELTIPIFATNSDCELIVKAMQ |
| | 1599) (aa) | GLLKDGNPIPSAIAANSGIYGGGGSSNNQLFAPGGRLSWGKGSSGGSGA |
| | | KPSDAASEAARPATSTLNRFSALQQAVPTESTDNRRVVQRSSLSRERGE |
| | | KAGDRGDRLERSERGGDRGDRLDRARTPATKRSFSKEVEERSRERPSQP |
| | | EGLRKAASLTEDRDRGRDAVKREAALPPVSPLKAALSEEELEKKSKAII |
| | | EEYLHLNDMKEAVQCVQELASPSLLFIFVRHGVESTLERSAIAREHMGQ |
| | | LLHQLLCAGHLSTAQYYQGLYEILELAEDMEIDIPHVWLYLAELVTPIL |
| | | QEGGVPMGELFREITKPLRPLGKAASLLLEILGLLCKSMGPKKVGTLWR |
| | | EAGLSWKEFLPEGQDIGAFVAEQKVEYTLGEESEAPGQRALPSEELNRQ |
| | | LEKLLKEGSSNQRVFDWIEANLSEQQIVSNTLVRALMTAVCYSAIIFETP |
| | | LRVDVAVLKARAKLLQKYLCDEQKELQALYALQALVVTLEQPPNLLR |
| | | MFFDALYDEDVVKEDAFYSWESSKDPAEQQGKGVALKSVTAFFKWLR |
| | | EAEEESDHN |
| 42 | MBP_eIF4G- | ATGGCCAGCAACTTCACCCAGTTCGTGCTGGTGGACAACGGCGGTA |
| | C1 (1080- | CCGGAGACGTGACCGTGGCCCCTTCTAACTTCGCCAACGGCATCGCC |
| | 1599) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAGC |
| | | AACAACCAGCTGTTCGCCCCAGGAGGAAGACTTAGCTGGGGCAAGG |
| | | GAAGTTCCGGCGGATCCGGCGCCAAGCCTTCCGACGCCGCCAGCGA |
| | | GGCTGCCAGACCTGCCACCAGCACCTTGAACCGCTTTTCCGCTCTGC |
| | | AGCAAGCTGTGCCGACCGAGAGCACCGACAACCGGCGAGTTGTGCA |
| | | GAGAAGCAGCCTGAGCAGAGAGAGGGGCGAGAAAGCCGGCGATAG |
| | | AGGTGACCGACTGGAGAGAAGCGAGAGAGGAGGTGATAGAGGCGA |
| | | CCGGCTTGACAGAGCCAGAACCCCTGCCACAAAGCGATCGTTCAGC |
| | | AAGGAGGTCGAAGAGAGGTCCAGGGAGCGCCCTAGCCAGCCGGAA |
| | | GGACTCAGAAAGGCAGCCAGTCTAACAGAGGACCGCGACAGGGGA |
| | | AGAGACGCCGTGAAGAGGGAGGCCGCACTGCCTCCTGTGAGCCCTC |
| | | TGAAGGCGGCACTGAGCGAAGAAGAACTTGAGAAGAAGAGTAAGG |
| | | CGATCATCGAGGAGTACCTGCACCTGAACGACATGAAGGAGGCCGT |
| | | GCAGTGCGTGCAAGAGCTCGCGAGCCCATCACTGCTGTTCATCTTCG |
| | | TCCGGCACGGCGTGGAGTCCACACTGGAAAGATCTGCCATTGCTAG |
| | | GGAGCATATGGGCCAGTTGTTGCACCAATTGCTTTGCGCCGGCCACC |
| | | TAAGTACTGCCCAGTACTATCAGGGTTTATACGAGATCCTCGAACTG |
| | | GCCGAAGACATGGAGATCGACATCCCTCACGTGTGGCTGTACCTCGC |
| | | AGAACTGGTGACCCCTATCCTGCAGGAGGGCGGCGTTCCAATGGGT |
| | | GAGTTGTTCAGAGAAATCACAAAGCCACTGCGCCCACTGGGCAAGG |
| | | CGGCTAGTCTCCTTCTGGAGATTCTCGGCCTGCTCTGTAAGAGCATG |
| | | GGTCCAAAGAAGGTGGGCACCCTGTGGAGGGAAGCTGGACTCTCCT |
| | | GGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTCAAGGAGGGATCCTCAAATCAGAGAGTGTTCGACTGGAT |
| | | CGAGGCCAATCTCAGCGAGCAGCAAATCGTGAGCAACACGTTGGTT |
| | | CGCGCTCTCATGACAGCCGTGTGCTACTCAGCCATTATCTTCGAGAC |
| | | GCCTCTCCGCGTGGACGTGGCAGTGCTCAAGGCCCGCGCTAAGCTGT |
| | | TACAGAAGTACCTGTGCGACGAGCAGAAGGAACTGCAGGCCCTGTA |
| | | CGCCCTGCAAGCTCTGGTCGTCACACTCGAGCAGCCTCCTAACCTGC |
| | | TGAGAATGTTCTTCGACGCATTGTACGACGAGGACGTTGTGAAGGA |
| | | AGACGCTTTCTACAGCTGGGAGTCTAGTAAGGATCCTGCGGAACAA |
| | | CAAGGCAAGGGCGTTGCTCTAAAGAGCGTGACCGCCTTCTTCAAGT |
| | | GGCTGCGAGAGGCTGAGGAAGAGTCGGACCACAAC |
| 43 | MBP_eIF4G- | MASNFTQFVLVDNGGTGDVTVAPSNFANGIAEWISSNSRSQAYKVTCS |
| | C2 (1080- | VRQSSAQNRKYTIKVEVPKGAWRSYLNMELTIPIFATNSDCELIVKAMQ |
| | 1451) (aa) | GLLKDGNPIPSAIAANSGIYGGGGSSNNQLFAPGGRLSWGKGSSGGSGA |
| | | KPSDAASEAARPATSTLNRFSALQQAVPTESTDNRRVVQRSSLSRERGE |
| | | KAGDRGDRLERSERGGDRGDRLDRARTPATKRSFSKEVEERSRERPSQP |
| | | EGLRKAASLTEDRDRGRDAVKREAALPPVSPLKAALSEEELEKKSKAII |
| | | EEYLHLNDMKEAVQCVQELASPSLLFIFVRHGVESTLERSAIAREHMGQ |
| | | LLHQLLCAGHLSTAQYYQGLYEILELAEDMEIDIPHVWLYLAELVTPIL |
| | | QEGGVPMGELFREITKPLRPLGKAASLLLEILGLLCKSMGPKKVGTLWR |
| | | EAGLSWKEFLPEGQDIGAFVAEQKVEYTLGEESEAPGQRALPSEELNRQ |
| | | LEKLL |
| 44 | MBP_eIF4G- | ATGGCCAGCAACTTCACCCAGTTCGTGCTGGTGGACAACGGCGGTA |
| | C2 (1080- | CCGGAGACGTGACCGTGGCCCCTTCTAACTTCGCCAACGGCATCGCC |
| | 1451) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAGC |
| | | AACAACCAGCTGTTCGCCCCAGGAGGAAGACTTAGCTGGGGCAAGG |
| | | GAAGTTCCGGCGGATCCGGCGCCAAGCCTTCCGACGCCGCCAGCGA |
| | | GGCTGCCAGACCTGCCACCAGCACCTTGAACCGCTTTTCCGCTCTGC |
| | | AGCAAGCTGTGCCGACCGAGAGCACCGACAACCGGCGAGTTGTGCA |
| | | GAGAAGCAGCCTGAGCAGAGAGAGGGGCGAGAAAGCCGGCGATAG |
| | | AGGTGACCGACTGGAGAGAAGCGAGAGAGGAGGTGATAGAGGCGA |
| | | CCGGCTTGACAGAGCCAGAACCCCTGCCACAAAGCGATCGTTCAGC |
| | | AAGGAGGTCGAAGAGAGGTCCAGGGAGCGCCCTAGCCAGCCGGAA |
| | | GGACTCAGAAAGGCAGCCAGTCTAACAGAGGACCGCGACAGGGGA |
| | | AGAGACGCCGTGAAGAGGGAGGCCGCACTGCCTCCTGTGAGCCCTC |
| | | TGAAGGCGGCACTGAGCGAAGAAGAACTTGAGAAGAAGAGTAAGG |
| | | CGATCATCGAGGAGTACCTGCACCTGAACGACATGAAGGAGGCCGT |
| | | GCAGTGCGTGCAAGAGCTCGCGAGCCCATCACTGCTGTTCATCTTCG |
| | | TCCGGCACGGCGTGGAGTCCACACTGGAAAGATCTGCCATTGCTAG |
| | | GGAGCATATGGGCCAGTTGTTGCACCAATTGCTTTGCGCCGGCCACC |
| | | TAAGTACTGCCCAGTACTATCAGGGTTTATACGAGATCCTCGAACTG |
| | | GCCGAAGACATGGAGATCGACATCCCTCACGTGTGGCTGTACCTCGC |
| | | AGAACTGGTGACCCCTATCCTGCAGGAGGGCGGCGTTCCAATGGGT |
| | | GAGTTGTTCAGAGAAATCACAAAGCCACTGCGCCCACTGGGCAAGG |
| | | CGGCTAGTCTCCTTCTGGAGATTCTCGGCCTGCTCTGTAAGAGCATG |
| | | GGTCCAAAGAAGGTGGGCACCCTGTGGAGGGAAGCTGGACTCTCCT |
| | | GGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTC |
| 45 | MBP-CTIF | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSENSSAASASSEAGSSRSQEIEELERF |
| | | IDSYVLEYQVQGLLADKTEGDGESERTQSHISQWTADCSEPLDSSCSFS |
| | | RGRAPPQQNGSKDNSLDMLGTDIWAANTFDSFSGATWDLQPEKLDFTQ |
| | | FHRKVRHTPKQPLPHIDREGCGKGKLEDGDGINLNDIEKVLPAWQGYH |
| | | PMPHEVELIAHTKKLFRRRRNDRRRQQRPPGGNKPQQHGDHQPGSAKH |
| | | NRDHQKSYQGGSAPHPSGRPTHHGYSQNRRWHHGNMKHPPGDKGEA |
| | | GAHRNAKETMTIENPKLEDTAGDTGHSSLEAPRSPDTLAPVASERLPPQ |
| | | QSGGPEVETKRKDSILPERIGERPKITLLQSSKDRLRRRLKEKDEVAVET |
| | | TTPQQNKMDKLIEILNSMRNNSSDVDTKLTTFMEEAQNSTNSEEMLGEI |
| | | VRTIYQKAVSDRSFAFTAAKLCDKMALFMVEGTKFRSLLLNMLQKDFT |
| | | VREELQQQDVERWLGFITFLCEVFGTMRSSTGEPFRVLVCPIYTCLRELL |
| | | QSQDVKEDAVLCCSMELQSTGRLLEEQLPEMMTELLASARDKMLCPSE |
| | | SMLTRSLLLEVIELHANSWNPLTPPITQYYNRTIQKLTA |
| 46 | MBP-CTIF | ATGGCCAGCAACTTCACCCAGTTCGTGCTGGTGGACAACGGCGGTA |
| | (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAG |
| | | AACAGTAGCGCCGCTAGCGCGAGCAGCGAAGCCGGGAGCAGCCGGT |
| | | CTCAGGAGATCGAGGAGCTGGAGCGGTTCATCGACAGCTACGTGCT |
| | | GGAGTACCAGGTGCAGGGCCTGCTGGCCGACAAGACCGAGGGCGAC |
| | | GGCGAGAGCGAGCGGACCCAGAGCCACATCAGCCAGTGGACCGCCG |
| | | ACTGCAGCGAGCCCCTGGACTCTAGCTGCTCATTCAGTCGTGGACGG |
| | | GCCCCTCCACAGCAGAACGGCAGCAAGGACAACAGCCTGGACATGC |
| | | TGGGCACCGACATCTGGGCCGCCAACACCTTCGACAGCTTCAGCGG |
| | | CGCCACCTGGGATCTGCAGCCCGAGAAGCTGGACTTTACCCAGTTCC |
| | | ACCGGAAGGTGCGGCACACTCCCAAGCAGCCCCTGCCCCACATCGA |
| | | TCGGGAGGGCTGCGGCAAGGGCAAGCTGGAAGACGGCGACGGCAT |
| | | CAACCTGAACGACATCGAGAAGGTGCTGCCTGCCTGGCAGGGCTAC |
| | | CACCCCATGCCCCACGAGGTGGAGATCGCCCACACCAAGAAGCTGT |
| | | TCCGGCGACGACGCAACGACCGGCGTAGGCAGCAACGGCCGCCTGG |
| | | AGGGAACAAGCCCCAGCAGCACGGAGACCACCAGCCCGGTAGCGCC |
| | | AAGCACAACCGGGACCACCAGAAGAGCTACCAGGGCGGAAGCGCA |
| | | CCACACCCCTCGGGCAGACCCACCCACCACGGCTACAGCCAGAACC |
| | | GGCGGTGGCATCACGGTAACATGAAGCACCCACCCGGCGACAAAGG |
| | | AGAGGCCGGCGCTCACCGTAACGCCAAGGAGACCATGACCATCGAG |
| | | AACCCCAAGCTGGAGGATACCGCCGGCGATACGGGTCACAGCAGCC |
| | | TGGAGGCACCGCGGTCTCCCGACACCCTGGCACCCGTGGCCAGCGA |
| | | ACGGCTGCCACCCCAACAGAGCGGCGGCCCTGAGGTTGAGACCAAG |
| | | CGGAAGGACAGCATCCTGCCCGAACGGATCGGTGAGCGGCCCAAGA |
| | | TCACCTTACTGCAGAGTAGCAAGGACCGGCTGAGACGGCGGCTGAA |
| | | GGAGAAGGACGAGGTGGCCGTGGAGACAACCACTCCCCAGCAGAA |
| | | CAAGATGGACAAGCTGATCGAGATCCTGAACAGCATGCGGAACAAC |
| | | AGCAGCGACGTGGACACCAAGCTGACCACCTTCATGGAGGAGGCCC |
| | | AGAACAGCACCAACAGCGAGGAGATGCTGGGCGAGATCGTGCGGA |
| | | CCATCTACCAGAAGGCCGTGAGCGACCGGAGCTTCGCCTTCACCGCC |
| | | GCCAAGCTGTGCGACAAGATGGCCCTGTTCATGGTGGAGGGCACCA |
| | | AGTTCCGGAGCTTACTGCTGAATATGCTGCAGAAGGACTTCACCGTG |
| | | CGGGAGGAGCTGCAGCAGCAGGACGTGGAGCGGTGGCTGGGCTTCA |
| | | TCACCTTCCTGTGCGAGGTGTTCGGCACCATGCGGAGCAGCACCGGC |
| | | GAACCCTTCCGGGTGCTGGTGTGCCCCATCTACACCTGCCTGCGGGA |
| | | GTTGCTGCAGAGCCAGGACGTGAAGGAGGACGCCGTGCTGTGCTGC |
| | | AGCATGGAACTGCAGAGCACTGGCCGGCTGCTGGAGGAGCAGCTGC |
| | | CCGAGATGATGACCGAGCTGCTCGCTAGCGCCCGGGACAAGATGCT |
| | | GTGCCCCAGCGAGAGCATGCTGACCCGGAGCCTGCTTCTGGAGGTG |
| | | ATCGAGCTGCACGCCAACAGCTGGAATCCCCTGACCCCTCCCATCAC |
| | | CCAGTACTACAACCGGACCATCCAGAAGCTGACCGCC |
| 47 | MBP- | MASNFTQFVLVDNGGTGDVTVAPSNFANGIAEWISSNSRSQAYKVTCS |
| | CTIF(379- | VRQSSAQNRKYTIKVEVPKGAWRSYLNMELTIPIFATNSDCELIVKAMQ |
| | 579) (aa) | GLLKDGNPIPSAIAANSGIYGGGGSLNSMRNNSSDVDTKLTTFMEEAQN |
| | | STNSEEMLGEIVRTIYQKAVSDRSFAFTAAKLCDKMALFMVEGTKFRSL |
| | | LLNMLQKDFTVREELQQQDVERWLGFITFLCEVFGTMRSSTGEPFRVL |
| | | VCPIYTCLRELLQSQDVKEDAVLCCSMELQSTGRLLEEQLPEMMTELLA |
| | | SARDKMLCPSESMLTRSLLLEVIELHANSWN |
| 48 | MBP- | ATGGCCAGCAACTTCACCCAGTTCGTGCTGGTGGACAACGGCGGTA |
| | CTIF(379- | CCGGAGACGTGACCGTGGCCCCTTCTAACTTCGCCAACGGCATCGCC |
| | 579) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCCTG |
| | | AACAGCATGAGAAACAACAGCAGCGACGTGGACACCAAGCTGACC |
| | | ACCTTCATGGAGGAGGCCCAGAACAGCACCAACAGCGAGGAGATGC |
| | | TGGGCGAGATCGTGAGAACCATCTACCAGAAGGCCGTGAGCGACAG |
| | | AAGCTTCGCCTTCACCGCCGCCAAGCTGTGCGACAAGATGGCCCTGT |
| | | TCATGGTGGAGGGCACCAAGTTCAGAAGCCTGCTGCTGAACATGCT |
| | | GCAGAAGGACTTCACCGTGAGAGAGGAGCTGCAGCAGCAGGACGTG |
| | | GAGCGATGGCTGGGCTTCATCACCTTCCTGTGCGAGGTGTTCGGCAC |
| | | CATGAGAAGCAGCACCGGCGAGCCTTTCAGAGTGCTGGTGTGCCCT |
| | | ATCTACACCTGCCTGAGAGAGCTGCTGCAGAGCCAGGACGTGAAGG |
| | | AGGACGCCGTGCTGTGCTGCAGCATGGAGCTGCAGAGCACCGGCAG |
| | | ACTGCTGGAGGAGCAGCTGCCTGAGATGATGACCGAGCTGCTGGCC |
| | | AGCGCCAGAGACAAGATGCTGTGCCCTAGCGAGAGCATGCTGACTA |
| | | GATCCCTACTGTTGGAGGTGATCGAGCTGCACGCCAACAGCTGGAA |
| | | C |
| 49 | MBP- | MASNFTQFVLVDNGGTGDVTVAPSNFANGIAEWISSNSRSQAYKVTCS |
| | CTIF(365- | VRQSSAQNRKYTIKVEVPKGAWRSYLNMELTIPIFATNSDCELIVKAMQ |
| | 598) (aa) | GLLKDGNPIPSAIAANSGIYGGGGSTTPQQNKMDKLIEILNSMRNNSSD |
| | | VDTKLTTFMEEAQNSTNSEEMLGEIVRTIYQKAVSDRSFAFTAAKLCDK |
| | | MALFMVEGTKFRSLLLNMLQKDFTVREELQQQDVERWLGFITFLCEVF |
| | | GTMRSSTGEPFRVLVCPIYTCLRELLQSQDVKEDAVLCCSMELQSTGRL |
| | | LEEQLPEMMTELLASARDKMLCPSESMLTRSLLLEVIELHANSWNPLTP |
| | | PITQYYNRTIQKLTA |
| 50 | MBP- | ATGGCCAGCAACTTCACCCAGTTCGTGCTGGTGGACAACGGCGGTA |
| | CTIF(365- | CCGGAGACGTGACCGTGGCCCCTTCTAACTTCGCCAACGGCATCGCC |
| | 598) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCACC |
| | | ACCCCTCAGCAGAACAAGATGGACAAGCTGATCGAGATCCTGAACA |
| | | GCATGAGAAACAACAGCAGCGACGTGGACACCAAGCTGACCACCTT |
| | | CATGGAGGAGGCCCAGAACAGCACCAACAGCGAGGAGATGCTGGG |
| | | CGAGATCGTGAGAACCATCTACCAGAAGGCCGTGAGCGACAGAAGC |
| | | TTCGCCTTCACCGCCGCCAAGCTGTGCGACAAGATGGCCCTGTTCAT |
| | | GGTGGAGGGCACCAAGTTCAGAAGCCTGCTGCTGAACATGCTGCAG |
| | | AAGGACTTCACCGTGAGAGAGGAGCTGCAGCAGCAGGACGTGGAG |
| | | AGGTGGCTGGGCTTCATCACCTTCCTGTGCGAGGTGTTCGGCACCAT |
| | | GAGAAGCAGCACCGGCGAGCCTTTCAGAGTGCTGGTGTGCCCTATCT |
| | | ACACCTGCCTGAGAGAGCTGCTGCAGAGCCAGGACGTGAAGGAGGA |
| | | CGCCGTGCTGTGCTGCAGCATGGAGCTGCAGAGCACCGGCAGACTG |
| | | CTGGAGGAGCAGCTGCCTGAGATGATGACCGAGCTGCTGGCCAGCG |
| | | CCAGAGACAAGATGCTGTGCCCTAGCGAGAGCATGCTGACGCGCAG |
| | | CCTCCTGCTGGAGGTGATCGAGCTGCACGCCAACAGCTGGAACCCTC |
| | | TGACCCCTCCTATCACCCAGTACTACAACAGAACCATCCAGAAGCTG |
| | | ACCGCC |
| 51 | MBP-eIF4G- | MASNFTQFVLVDNGGTGDVTVAPSNFANGIAEWISSNSRSQAYKVTCS |
| | mid4 (752- | VRQSSAQNRKYTIKVEVPKGAWRSYLNMELTIPIFATNSDCELIVKAMQ |
| | 993) (aa) | GLLKDGNPIPSAIAANSGIYGGGGSADGSKTQDLFRRVRSILNKLTPQMF |
| | | QQLMKQVTQLAIDTEERLKGVIDLIFEKAISEPNFSVAYANMCRCLMAL |
| | | KVPTTEKPTVTVNFRKLLLNRCQKEFEKDKDDDEVFEKKQKEMDEAA |
| | | TAEERGRLKEELEEARDIARRRSLGNIKFIGELFKLKMLTEAIMHDCVV |
| | | KLLKNHDEESLECLCRLLTTIGKDLDFEKAKPRMDQYFNQMEKIIKEKK |
| | | TSSRIRFMLQDVLDLRGSNWVPR |
| 52 | MBP-eIF4G- | ATGGCCAGCAACTTCACCCAGTTCGTGCTGGTGGACAACGGCGGTA |
| | mid4 (752- | CCGGAGACGTGACCGTGGCCCCTTCTAACTTCGCCAACGGCATCGCC |
| | 993) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGCC |
| | | GACGGCAGCAAGACCCAGGACCTGTTCAGAAGAGTGAGAAGCATCC |
| | | TGAACAAGCTGACCCCTCAGATGTTCCAGCAGCTGATGAAGCAGGT |
| | | GACCCAGCTGGCCATCGACACCGAGGAGAGACTGAAGGGCGTGATC |
| | | GACCTGATCTTCGAGAAGGCCATCAGCGAGCCTAACTTCAGCGTGG |
| | | CCTACGCCAACATGTGCCGGTGCCTGATGGCCCTGAAGGTGCCTACC |
| | | ACCGAGAAGCCTACCGTGACCGTGAACTTCAGAAAGCTGCTGCTGA |
| | | ACCGGTGCCAGAAGGAGTTCGAGAAGGACAAGGACGACGACGAGG |
| | | TGTTCGAGAAGAAGCAGAAGGAGATGGACGAGGCCGCCACCGCCG |
| | | AGGAGAGAGGCAGACTGAAGGAGGAGCTGGAGGAGGCCAGAGACA |
| | | TCGCCAGAAGAAGAAGCCTGGGCAACATCAAGTTCATCGGCGAGCT |
| | | GTTCAAGCTGAAGATGCTGACCGAGGCCATCATGCACGACTGCGTG |
| | | GTGAAGCTGCTGAAGAACCACGACGAGGAGAGCCTGGAGTGCCTGT |
| | | GCAGACTGCTGACCACCATCGGCAAGGACCTGGATTTCGAGAAGGC |
| | | GAAGCCTAGAATGGACCAGTACTTCAACCAGATGGAGAAGATCATC |
| | | AAGGAGAAGAAGACCAGCAGCAGAATCAGATTCATGCTGCAGGAC |
| | | GTGCTGGACCTGAGAGGCAGCAACTGGGTGCCTAGA |
| 53 | MBP-LacZ | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSSFTLTNKNVIFVAGLGGIGLDTSKE |
| | | LLKRDPVVLQRRDWENPGVTQLNRLAAHPPFASWRNSEEARTDRPSQQ |
| | | LRSLNGEWRFAWFPAPEAVPESWLECDLPEADTVVVPSNWQMHGYDA |
| | | PIYTNVTYPITVNPPFVPTENPTGCYSLTFNVDESWLQEGQTRIIFDGVNS |
| | | AFHLWCNGRWVGYGQDSRLPSEFDLSAFLRAGENRLAVMVLRWSDGS |
| | | YLEDQDMWRMSGIFRDVSLLHKPTTQISDFHVATRENDDFSRAVLEAE |
| | | VQMCGELRDYLRVTVSLWQGETQVASGTAPFGGEIIDERGGYADRVTL |
| | | RLNVENPKLWSAEIPNLYRAVVELHTADGTLIEAEACDVGFREVRIENG |
| | | LLLLNGKPLLIRGVNRHEHHPLHGQVMDEQTMVQDILLMKQNNFNAV |
| | | RCSHYPNHPLWYTLCDRYGLYVVDEANIETHGMVPMNRLTDDPRWLP |
| | | AMSERVTRMVQRDRNHPSVIIWSLGNESGHGANHDALYRWIKSVDPSR |
| | | PVQYEGGGADTTATDIICPMYARVDEDQPFPAVPKWSIKKWLSLPGET |
| | | RPLILCEYAHAMGNSLGGFAKYWQAFRQYPRLQGGFVWDWVDQSLIK |
| | | YDENGNPWSAYGGDFGDTPNDRQFCMNGLVFADRTPHPALTEAKHQQ |
| | | QFFQFRLSGQTIEVTSEYLFRHSDNELLHWMVALDGKPLASGEVPLDV |
| | | APQGKQLIELPELPQPESAGQLWLTVRVVQPNATAWSEAGHISAWQQ |
| | | WRLAENLSVTLPAASHAIPHLTTSEMDFCIELGNKRWQFNRQSGFLSQ |
| | | MWIGDKKQLLTPLRDQFTRAPLDNDIGVSEATRIDPNAWVERWKAAG |
| | | HYQAEAALLQCTADTLADAVLITTAHAWQHQGKTLFISRKTYRIDGSG |
| | | QMAITVDVEVASDTPHPARIGLNCQLAQVAERVNWLGLGPQENYPDRL |
| | | TAACFDRWDLPLSDMYTPYVFPSENGLRCGTRELNYGPHQWRGDFQF |
| | | NISRYSQQQLMETSHRHLLHAEEGTWLNIDGFHMGIGGDDSWSPSVSA |
| | | ELQLSAGRYHYQLVWCQK |
| 54 | MBP-LacZ | ATGGCCAGCAACTTCACCCAGTTCGTGCTGGTGGACAACGGCGGTA |
| | (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCTCG |
| | | TTCACGCTAACGAACAAGAACGTCATCTTCGTAGCGGGACTTGGCG |
| | | GTATCGGCCTAGACACGTCGAAGGAACTACTAAAGCGTGACCCGGT |
| | | AGTCCTCCAACGTCGCGATTGGGAGAACCCGGGCGTAACGCAACTA |
| | | AACCGTCTTGCGGCGCACCCGCCGTTTGCGTCGTGGCGTAACTCGGA |
| | | GGAGGCGCGAACGGATCGTCCGTCGCAACAACTACGTTCGCTCAAC |
| | | GGGGAGTGGCGCTTCGCGTGGTTCCCGGCGCCGGAGGCGGTACCGG |
| | | AGTCGTGGCTCGAGTGCGATCTACCGGAGGCGGACACGGTCGTCGT |
| | | ACCGTCGAACTGGCAAATGCACGGTTACGACGCGCCGATATACACG |
| | | AACGTCACGTACCCGATAACGGTAAACCCGCCGTTCGTCCCGACGG |
| | | AGAACCCGACGGGGTGCTACTCGCTAACGTTCAACGTTGACGAGTC |
| | | GTGGTTGCAAGAGGGTCAAACGCGTATCATATTCGACGGTGTAAAC |
| | | TCGGCGTTCCACCTGTGGTGCAACGGGCGCTGGGTAGGGTACGGCC |
| | | AAGACTCGCGTCTACCGTCGGAGTTCGACCTATCGGCGTTCCTACGA |
| | | GCGGGTGAGAACCGGCTAGCGGTCATGGTCCTACGTTGGTCGGACG |
| | | GTTCGTACCTCGAGGACCAAGACATGTGGCGAATGTCGGGTATCTTC |
| | | CGCGACGTATCGCTCCTACACAAGCCGACGACGCAAATCTCGGACTT |
| | | CCACGTCGCGACGCGTTTCAACGACGATTTCTCGCGGGCAGTCCTAG |
| | | AGGCGGAGGTCCAAATGTGCGGGGAGCTACGTGACTACCTCCGTGT |
| | | CACGGTATCGCTCTGGCAAGGTGAGACGCAAGTAGCGTCGGGTACG |
| | | GCGCCGTTCGGCGGTGAGATCATCGACGAGCGTGGTGGGTACGCGG |
| | | ACCGTGTAACGCTACGTCTAAACGTCGAGAACCCGAAGCTCTGGTC |
| | | GGCGGAGATCCCGAACCTATACCGTGCGGTCGTCGAGCTACATACG |
| | | GCGGACGGGACGCTAATAGAGGCGGAAGCGTGCGACGTCGGGTTTC |
| | | GAGAGGTTCGTATAGAGAACGGGCTGCTACTTCTAAACGGGAAGCC |
| | | GTTGCTCATACGTGGTGTCAACCGTCACGAGCACCACCCGCTACACG |
| | | GTCAAGTAATGGACGAGCAAACGATGGTACAAGACATCCTACTAAT |
| | | GAAGCAGAACAACTTCAACGCGGTACGCTGTTCGCATTACCCGAAC |
| | | CATCCGTTGTGGTACACGCTTTGCGACCGATACGGTCTATACGTCGT |
| | | AGACGAGGCGAACATAGAGACGCACGGGATGGTACCGATGAATCGC |
| | | CTAACGGACGACCCGCGTTGGCTACCGGCGATGTCGGAGCGAGTCA |
| | | CGCGTATGGTCCAACGGGACCGTAACCACCCGTCGGTAATAATCTG |
| | | GTCGCTAGGCAACGAATCGGGGCACGGGGCGAACCACGACGCGCTA |
| | | TACCGTTGGATCAAGTCGGTAGACCCGTCGCGTCCGGTACAATACGA |
| | | AGGTGGCGGTGCGGACACGACGGCGACGGACATCATCTGCCCGATG |
| | | TACGCGCGCGTCGACGAAGACCAACCGTTCCCGGCGGTACCGAAGT |
| | | GGTCGATCAAGAAGTGGCTCTCGTTGCCGGGTGAAACGCGTCCGTTG |
| | | ATACTTTGCGAGTACGCGCACGCGATGGGCAACTCGTTGGGTGGGTT |
| | | CGCGAAGTACTGGCAGGCGTTCCGTCAATACCCGCGTCTACAGGGT |
| | | GGGTTCGTCTGGGACTGGGTAGACCAATCGCTAATCAAGTACGACG |
| | | AGAACGGCAACCCGTGGTCGGCGTACGGTGGGGACTTCGGGGACAC |
| | | GCCGAACGACCGCCAATTCTGTATGAACGGCCTAGTCTTCGCGGACC |
| | | GAACGCCGCACCCGGCGTTGACGGAGGCGAAGCATCAACAACAATT |
| | | CTTCCAATTCCGTCTATCGGGGCAAACGATCGAGGTAACGTCGGAGT |
| | | ACTTGTTCCGGCACTCGGACAACGAGCTACTACACTGGATGGTAGCA |
| | | CTAGACGGCAAGCCGCTAGCGTCGGGAGAAGTCCCTTTGGACGTCG |
| | | CGCCGCAAGGTAAGCAACTAATCGAGCTACCGGAGCTACCGCAACC |
| | | GGAGTCGGCGGGTCAACTGTGGTTGACGGTCCGTGTCGTTCAACCGA |
| | | ACGCGACGGCGTGGTCGGAGGCGGGTCACATCTCGGCGTGGCAGCA |
| | | GTGGCGTCTAGCGGAGAACCTCTCGGTCACGCTACCGGCGGCGTCG |
| | | CACGCGATACCGCATCTAACGACGTCGGAGATGGACTTCTGCATCG |
| | | AGTTGGGGAACAAGAGGTGGCAGTTCAACCGTCAATCGGGATTCCT |
| | | ATCGCAAATGTGGATAGGTGACAAGAAGCAACTACTAACGCCGCTA |
| | | CGTGATCAGTTCACGCGTGCTCCGCTAGACAACGACATAGGTGTTTC |
| | | GGAGGCGACGCGTATAGACCCGAACGCGTGGGTGGAGCGGTGGAA |
| | | GGCGGCGGGGCACTACCAAGCGGAGGCGGCGCTACTACAGTGCACG |
| | | GCGGACACGCTAGCGGACGCGGTATTGATCACGACGGCGCACGCGT |
| | | GGCAACACCAGGGGAAGACGCTATTCATCTCGCGTAAGACGTACCG |
| | | TATCGACGGTTCGGGCCAAATGGCGATCACGGTCGACGTAGAGGTA |
| | | GCGTCGGACACGCCGCATCCGGCGCGCATCGGTCTAAACTGCCAAC |
| | | TAGCGCAAGTAGCGGAGCGTGTAAACTGGCTAGGGCTAGGGCCGCA |
| | | AGAGAACTATCCGGACCGCCTAACGGCGGCGTGCTTCGACCGTTGG |
| | | GACCTACCGCTTTCGGACATGTATACCCCGTACGTCTTCCCGTCGGA |
| | | GAACGGGTTGAGGTGCGGGACGCGCGAGCTAAACTACGGGCCGCAC |
| | | CAGTGGCGAGGGGACTTCCAATTCAACATATCGCGTTACTCGCAACA |
| | | ACAACTAATGGAGACGTCGCACCGTCACCTACTACACGCGGAGGAG |
| | | GGGACGTGGCTAAACATCGACGGGTTCCACATGGGCATAGGTGGGG |
| | | ACGACTCGTGGTCGCCGTCGGTCTCGGCGGAGCTCCAACTCTCGGCG |
| | | GGTCGTTACCATTACCAACTAGTTTGGTGCCAGAAG |
| 55 | 50S | MYVRFEVPEDMQNEALSLLEKVRESGKVKKGTNETTKAVERGLAKLV |
| | ribosomal | YIAEDVDPPEIVAHLPLLCEEKNVPYIYVKSKNDLGRAVGIEVPCASAAI |
| | L7Ae protein | INEGELRKELGSLVEKIKGLQK |
| | (aa) | |
| 56 | 50S | ATGTACGTGAGATTTGAGGTTCCTGAGGACATGCAGAACGAAGCTC |
| | ribosomal | TGAGTCTGCTGGAGAAGGTTAGGGAGAGCGGTAAGGTAAAGAAAG |
| | L7Ae protein | GTACCAACGAGACGACAAAGGCTGTGGAGAGGGGACTGGCAAAGC |
| | (nt) | TCGTTTACATCGCAGAGGATGTTGACCCGCCTGAGATCGTTGCTCAT |
| | | CTGCCCCTCCTCTGCGAGGAGAAGAATGTGCCGTACATTTACGTTAA |
| | | AAGCAAGAACGACCTTGGAAGGGCTGTGGGCATTGAGGTGCCATGC |
| | | GCTTCGGCAGCGATAATCAACGAGGGAGAGCTGAGAAAGGAGCTTG |
| | | GAAGCCTTGTGGAGAAGATTAAAGGCCTTCAGAAGAGATCTCATAT |
| | | GCATCTCGAG |
| 57 | Pre- | MASNFTQFVLVDNGGTGDVTVAPSNFANGVAEWISSNSRSQAYKVTCS |
| | dimerized | VRQSSAQNRKYTIKVEVPKVATQTVGGVELPVAAWRSYLNMELTIPIF |
| | MBP (aa) | ATNSDCELIVKAMQGLLKDGNPIPSAIAANSGIYGGGGSASNFTQFVLV |
| | (linker | DNGGTGDVTVAPSNFANGVAEWISSNSRSQAYKVTCSVRQSSAQNRK |
| | sequence | YTIKVEVPKVATQTVGGVELPVAAWRSYLNMELTIPIFATNSDCELIVK |
| | underlined) | AMQGLLKDGNPIPSAIAANSGIY |
| 58 | Pre- | ATGGCCAGCAATTTTACCCAGTTCGTGCTGGTCGACAACGGCGG |
| | dimerized | GACCGGTGACGTTACCGTGGCCCCTAGCAATTTCGCTAACGGCGTGG |
| | MBP (nt) | CTGAGTGGATCTCCAGCAACAGCCGGAGCCAAGCTTACAAGGTGAC |
| | (linker | CTGTAGCGTGCGGCAGAGCAGCGCCCAGAACCGGAAGTACACCATC |
| | sequence | AAGGTAGAAGTGCCTAAGGTGGCCACACAGACTGTGGGCGGTGTCG |
| | underlined) | AGCTGCCCGTGGCAGCTTGGAGGAGCTATCTGAATATGGAACTGAC |
| | | CATCCCCATCTTCGCCACTAACAGCGATTGTGAGCTGATTGTTAAGG |
| | | CCATGCAAGGCCTGCTAAAGGACGGGAACCCCATTCCCAGCGCCAT |
| | | CGCTGCCAACAGCGGCATCTACGGAGGAGGCGGAAGCGCTAGCAAC |
| | | TTCACCCAGTTTGTGCTGGTGGACAACGGTGGCACCGGCGACGTGAC |
| | | CGTGGCTCCCAGCAACTTTGCCAACGGTGTGGCCGAGTGGATCAGC |
| | | AGCAATTCTCGGAGCCAGGCCTACAAGGTCACGTGCAGCGTGCGCC |
| | | AAAGCAGCGCTCAGAATCGGAAGTATACTATCAAGGTGGAGGTGCC |
| | | CAAAGTGGCCACCCAGACCGTGGGTGGCGTGGAGCTGCCTGTGGCC |
| | | GCTTGGCGATCATACCTGAACATGGAGCTCACCATCCCTATCTTCGC |
| | | AACCAACAGCGACTGCGAGCTGATCGTGAAAGCCATGCAGGGCCTG |
| | | CTGAAGGACGGCAATCCCATCCCCAGCGCTATCGCCGCCAATAGCG |
| | | GAATCTAC |
| 59 | MBP-mid2- | MASNFTQFVLVDNGGTGDVTVAPSNFANGIAEWISSNSRSQAYKVTCS |
| | mut (aa) | VRQSSAQNRKYTIKVEVPKGAWRSYLNMELTIPIFATNSDCELIVKAMQ |
| | | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKAIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAV |
| 60 | MBP-mid2- | ATGGCCAGCAACTTCACCCAGTTCGTGCTGGTGGACAACGGCGGTA |
| | mut (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGGCGATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTG |
| 61 | PCP (aa) | MSKTIVLSVGEATRTLTEIQSTADRQIFEEKVGPLVGRLRLTASLRQNGA |
| | | KTAYRVNLKLDQADVVDCSTSVCGELPKVRYTQVWSHDVTIVANSTE |
| | | ASRKSLYDLTKSLVVQATSEDLVVNLVPLGR |
| 62 | PCP (nt) | ATGAGCAAGACCATCGTGCTGAGCGTGGGCGAGGCCACCAGAACCC |
| | | TGACCGAGATCCAGAGCACCGCCGACAGACAGATCTTCGAGGAGAA |
| | | GGTGGGCCCTCTGGTGGGCAGACTGAGACTGACCGCCAGCCTGAGA |
| | | CAGAACGGCGCCAAGACCGCCTACAGAGTGAACCTGAAGCTGGACC |
| | | AGGCCGACGTGGTGGACTGCAGCACCAGCGTGTGCGGCGAGCTGCC |
| | | TAAGGTGAGATACACCCAGGTGTGGAGCCACGACGTGACCATCGTG |
| | | GCCAACAGCACCGAGGCCAGCAGAAAGAGCCTGTACGACCTGACCA |
| | | AGAGCCTGGTGGTGCAGGCCACCAGCGAGGACCTGGTGGTGAACCT |
| | | GGTGCCTCTGGGCAGA |
| 63 | PUF (aa) | MGSSHHHHHHSQDLEVLFQGPHMGRSRLLEDFRNNRYPNLQLREIAGH |
| | | IMEFSQDQHGSRFIQLKLERATPAERQLVFNEILQAAYQLMVDVFGSYV |
| | | IEKFFEFGSLEQKLALAERIRGHVLSLALQMYGSRVIEKALEFIPSDQQN |
| | | EMVRELDGHVLKCVKDQNGCHVVQKCIECVQPQSLQFIIDAFKGQVFA |
| | | LSTHPYGSRVIERILEHCLPDQTLPILEELHQHTEQLVQDQYGCYVIQHV |
| | | LEHGRPEDKSKIVAEIRGNVLVLSQHKFASNVVEKCVTHASRTERAVLI |
| | | DEVCTMNDGPHSALYTMMKDQYASYVVEKMIDVAEPGQRKIVMHKIR |
| | | PHIMEFSQDQHGSRFIELKLERATPAERQLVFNEILQAAYQLMVDVFGC |
| | | YVIQKFFEFGSLEQKLALAERIRGHVLSLALQMYGSRVIEKALEFIPSDQ |
| | | QNEMVRELDGHVLKCVKDQNGCHVVQKCIECVQPQSLQFIIDAFKGQV |
| | | FALSTHPYGSRVIERILEHCLPDQTLPILEELHQHTEQLVQDQYGCYVIQ |
| | | HVLEHGRPEDKSKIVAEIRGNVLVLSQHKFASNVVEKCVTHASRTERA |
| | | VLIDEVCTMNDGPHSALYTMMKDQYACYVVQKMIDVAEPGQRKIVM |
| | | HKIRPHIATLRKYTYGKHILAKLEKYYMKNGVDLG |
| 64 | PUF (nt) | ATGGGCAGCAGCCACCATCACCACCACCACAGCCAGGACCTGGAGG |
| | | TGCTGTTCCAGGGCCCACACATGGGCCGTAGCCGGCTGCTGGAGGA |
| | | CTTCCGGAACAACCGGTACCCCAACCTGCAGCTGCGGGAGATCGCC |
| | | GGCCACATCATGGAGTTCAGCCAGGACCAGCACGGCAGCCGCTTCA |
| | | TCCAGCTGAAGTTGGAGCGGGCTACACCCGCTGAGCGGCAGCTGGT |
| | | GTTCAACGAGATCCTGCAGGCCGCCTATCAACTGATGGTAGACGTGT |
| | | TCGGCAGCTACGTGATCGAGAAGTTCTTTGAGTTCGGCAGTCTGGAG |
| | | CAGAAGCTCGCCCTGGCCGAACGGATACGGGGCCACGTGCTTAGCC |
| | | TGGCCCTACAGATGTACGGCAGCAGGGTGATCGAGAAAGCCCTGGA |
| | | GTTCATCCCCAGCGACCAACAGAACGAAATGGTGCGGGAGCTCGAC |
| | | GGCCACGTCCTGAAGTGCGTGAAGGACCAGAACGGTTGCCACGTGG |
| | | TGCAGAAGTGCATCGAGTGCGTCCAGCCCCAGTCTCTGCAGTTCATT |
| | | ATCGACGCCTTCAAGGGCCAAGTGTTCGCCCTGAGTACCCACCCCTA |
| | | CGGAAGCCGGGTGATCGAACGGATTCTGGAGCATTGCCTGCCCGAT |
| | | CAGACCCTGCCCATCCTGGAGGAGCTGCATCAACACACCGAACAGC |
| | | TTGTGCAAGACCAGTACGGCTGCTACGTGATCCAGCACGTGTTGGAG |
| | | CACGGACGGCCCGAGGACAAGAGCAAGATTGTGGCCGAGATCAGG |
| | | GGTAACGTGCTGGTGCTGTCTCAGCACAAATTCGCCAGCAACGTGGT |
| | | AGAGAAGTGCGTGACCCACGCCAGCCGGACTGAACGGGCCGTGCTG |
| | | ATCGACGAGGTGTGCACTATGAACGACGGGCCCCACTCTGCCCTGTA |
| | | CACAATGATGAAAGACCAATACGCCAGCTACGTGGTGGAGAAGATG |
| | | ATCGACGTGGCCGAGCCAGGCCAACGGAAGATCGTGATGCATAAGA |
| | | TCCGGCCTCATATCATGGAGTTTAGCCAGGATCAACACGGCAGCCG |
| | | GTTCATCGAGCTGAAGCTGGAACGGGCCACCCCTGCCGAACGGCAG |
| | | CTGGTCTTTAACGAGATACTGCAAGCCGCCTACCAGCTGATGGTGGA |
| | | CGTGTTTGGTTGCTACGTTATCCAGAAGTTCTTCGAGTTCGGAAGCC |
| | | TGGAGCAGAAACTGGCCCTGGCCGAGCGTATCCGGGGCCACGTTCT |
| | | GAGCTTAGCCCTGCAGATGTACGGGAGCCGGGTTATCGAGAAGGCC |
| | | CTGGAATTCATCCCTAGCGATCAGCAGAACGAGATGGTCAGAGAGC |
| | | TGGACGGCCACGTGCTCAAGTGCGTGAAAGACCAGAACGGCTGCCA |
| | | CGTGGTACAGAAGTGTATCGAGTGTGTGCAGCCCCAGAGCCTCCAG |
| | | TTTATCATTGACGCCTTTAAGGGCCAGGTGTTTGCCCTGAGCACACA |
| | | CCCCTACGGCAGCCGGGTAATCGAGCGGATCCTGGAGCACTGCCTG |
| | | CCCGACCAGACCTTGCCCATCCTCGAGGAACTGCACCAGCACACCG |
| | | AGCAGCTGGTGCAGGACCAATACGGCTGTTACGTCATCCAGCACGTT |
| | | CTGGAGCACGGCCGGCCTGAGGACAAGTCTAAGATCGTGGCCGAAA |
| | | TCCGGGGCAACGTGTTGGTGCTGAGCCAGCACAAGTTCGCCTCAAA |
| | | CGTCGTGGAGAAGTGTGTGACTCACGCCTCTCGGACCGAGCGGGCA |
| | | GTGCTGATTGACGAGGTGTGTACCATGAACGACGGCCCACACAGCG |
| | | CCCTGTACACCATGATGAAGGACCAGTACGCCTGCTACGTTGTGCAG |
| | | AAGATGATTGACGTGGCCGAACCCGGTCAGCGCAAGATTGTGATGC |
| | | ACAAGATCCGGCCCCACATCGCCACCCTGCGGAAGTACACCTACGG |
| | | CAAGCACATCCTGGCCAAGCTGGAGAAGTACTACATGAAGAACGGC |
| | | GTGGACCTGGGC |
| 65 | PUF2 (aa) | MGSSHHHHHHSQDLEVLFQGPHMGRSRLLEDFRNNRYPNLQLREIAGH |
| | | IMEFSQDQHGSRFIQLKLERATPAERQLVFNEILQAAYQLMVDVFGNYV |
| | | IQKFFEFGSLEQKLALAERIRGHVLSLALQMYGNRVIQKALEFIPSDQQN |
| | | EMVRELDGHVLKCVKDQNGCHVVQKCIECVQPQSLQFIIDAFKGQVFA |
| | | LSTHPYGNRVIQRILEHCLPDQTLPILEELHQHTEQLVQDQYGCYVIQHV |
| | | LEHGRPEDKSKIVAEIRGNVLVLSQHKFASNVVEKCVTHASRTERAVLI |
| | | DEVCTMNDGPHSALYTMMKDQY ANYVVQKMIDVAEPGQRKIVMHKI |
| | | RPHIMEFSQDQHGNRFIQLKLERATPAERQLVFNEILQAAYQLMVDVFG |
| | | NYVIQKFFEFGSLEQKLALAERIRGHVLSLALQMYGNRVIQKALEFIPSD |
| | | QQNEMVRELDGHVLKCVKDQNGNHVVQKCIECVQPQSLQFIIDAFKGQ |
| | | VFALSTHPYGNRVIQRILEHCLPDQTLPILEELHQHTEQLVQDQYGNYVI |
| | | QHVLEHGRPEDKSKIVAEIRGNVLVLSQHKFANNVVQKCVTHASRTER |
| | | AVLIDEVCTMNDGPHSALYTMMKDQYANYVVQKMIDVAEPGQRKIV |
| | | MHKIRPHIATLRKYTYGKHILAKLEKYYMKNGVDLG |
| 66 | PUF2 (nt) | ATGGGCAGCTCCCATCACCACCACCATCATTCGCAGGACCTTGAAGT |
| | | CCTGTTCCAAGGTCCTCATATGGGTAGATCAAGGCTTTTGGAAGATT |
| | | TTCGAAATAACCGGTATCCTAACCTGCAGCTCAGGGAGATAGCAGG |
| | | ACACATCATGGAGTTCAGCCAGGACCAACACGGAAGCAGGTTTATC |
| | | CAACTGAAACTCGAGAGAGCAACGCCCGCAGAGCGTCAGCTGGTGT |
| | | TTAACGAGATCCTGCAGGCAGCCTACCAGCTGATGGTTGACGTGTTC |
| | | GGTAACTACGTAATTCAGAAGTTCTTTGAGTTCGGTTCTCTGGAACA |
| | | GAAACTCGCTCTGGCTGAGCGAATTCGGGGTCACGTGCTCTCACTGG |
| | | CGCTCCAGATGTACGGCAATAGAGTGATTCAGAAGGCTCTGGAATTT |
| | | ATTCCATCGGACCAGCAGAACGAAATGGTACGAGAACTGGACGGTC |
| | | ACGTCCTTAAGTGTGTCAAAGATCAGAACGGCTGTCACGTAGTACA |
| | | GAAGTGCATCGAGTGTGTGCAGCCGCAGAGCCTCCAGTTTATCATCG |
| | | ACGCCTTTAAAGGGCAGGTCTTCGCCTTGTCCACCCATCCGTACGGA |
| | | AACCGAGTCATTCAGCGCATCCTGGAACATTGCCTCCCGGATCAGAC |
| | | CCTGCCGATTCTGGAGGAGCTTCACCAGCACACCGAGCAACTAGTG |
| | | CAGGATCAGTACGGCTGTTACGTGATACAGCACGTCCTGGAGCACG |
| | | GTAGACCAGAGGACAAATCGAAGATTGTGGCCGAAATCAGAGGAA |
| | | ACGTGCTGGTCCTTTCCCAGCACAAGTTCGCTTCCAACGTGGTGGAG |
| | | AAGTGCGTGACACACGCAAGCCGCACCGAGAGGGCAGTCTTAATCG |
| | | ACGAAGTGTGCACAATGAACGACGGTCCACACTCTGCCTTATACAC |
| | | AATGATGAAGGACCAGTACGCAAACTACGTGGTGCAGAAGATGATT |
| | | GACGTAGCTGAGCCTGGCCAGCGAAAGATTGTCATGCATAAGATCA |
| | | GGCCTCATATTATGGAGTTTTCTCAGGATCAACACGGCAATCGGTTC |
| | | ATCCAATTAAAGCTGGAGAGGGCCACACCTGCGGAGAGGCAGTTGG |
| | | TCTTTAACGAAATTCTACAAGCCGCTTACCAATTAATGGTGGACGTC |
| | | TTTGGCAACTACGTGATCCAGAAATTCTTCGAGTTTGGAAGTCTTGA |
| | | ACAGAAGCTGGCGCTGGCTGAAAGAATCAGAGGTCACGTCCTCAGC |
| | | CTGGCATTGCAAATGTACGGAAATCGAGTCATCCAGAAAGCCCTTG |
| | | AGTTTATCCCCAGCGATCAACAGAACGAGATGGTTCGCGAATTGGA |
| | | CGGGCACGTGCTGAAGTGTGTTAAGGACCAGAACGGGAACCACGTC |
| | | GTGCAGAAGTGCATAGAGTGCGTCCAGCCACAGTCCCTGCAGTTTAT |
| | | TATTGACGCATTTAAGGGACAAGTGTTCGCCTTATCTACCCACCCTT |
| | | ACGGCAATAGGGTCATTCAGAGGATCTTGGAACACTGTCTTCCCGAC |
| | | CAGACACTGCCAATATTGGAAGAACTGCATCAACATACCGAACAGC |
| | | TCGTACAAGATCAGTACGGTAATTACGTCATTCAACACGTTCTCGAA |
| | | CACGGAAGGCCCGAAGACAAGTCTAAGATCGTCGCAGAGATACGTG |
| | | GGAACGTCCTCGTCTTAAGTCAGCATAAGTTCGCAAACAACGTTGTG |
| | | CAGAAGTGTGTAACACACGCGAGCAGAACAGAGAGAGCCGTGCTGA |
| | | TCGACGAGGTGTGTACCATGAACGACGGGCCACACAGCGCCCTCTA |
| | | CACCATGATGAAAGACCAATACGCTAACTACGTGGTTCAGAAGATG |
| | | ATCGACGTCGCCGAACCAGGTCAACGGAAGATCGTGATGCACAAGA |
| | | TCAGGCCACACATAGCCACCCTGAGAAAGTATACGTACGGTAAACA |
| | | TATCCTGGCAAAGTTAGAGAAGTACTATATGAAGAACGGCGTGGAC |
| | | TTAGGA |
| 67 | Lambda (aa) | MGNARTRRRERRAEKQAQWKAAN |
| 68 | Lambda (nt) | ATGGGCAACGCCCGGACCAGGAGAAGAGAGCGGCGGGCCGAGAAG |
| | | CAGGCCCAGTGGAAGGCCGCCAAC |
| 69 | U1A (aa) | MAVPETRPNHTIYINNLNEKIKKDELKKSLYAIFSQFGQILDILVSRSLK |
| | | MRGQAFVIFKEVSSATNALRSMQGFPFYDKPMRIQYAKTDSDIIAKMK |
| | | GTF |
| 70 | U1A (nt) | ATGGCCGTGCCTGAGACCAGACCTAACCACACCATCTACATCAACA |
| | | ACCTGAACGAGAAGATCAAGAAGGACGAGCTGAAGAAGAGCCTGT |
| | | ACGCCATCTTCAGCCAGTTCGGCCAGATCCTGGACATCCTGGTGAGC |
| | | AGAAGCCTGAAGATGAGAGGCCAGGCCTTCGTGATCTTCAAGGAGG |
| | | TGAGCAGCGCCACCAACGCCCTGAGAAGCATGCAGGGCTTCCCTTTC |
| | | TACGACAAGCCTATGAGAATCCAGTACGCCAAGACCGACAGCGACA |
| | | TCATCGCCAAGATGAAGGGCACCTTC |
| 71 | 15.5kd (aa) | MTEADVNPKAYPLADAHLTKKLLDLVQQSCNYKQLRKGANEATKTLN |
| | | RGISEFIVMAADAEPLEIILHLPLLCEDKNVPYVFVRSKQALGRACGVSR |
| | | PVIACSVTIKEGSQLKQQIQSIQQSIERLLV |
| 72 | 15.5kd (nt) | ATGACCGAGGCCGACGTGAACCCTAAGGCCTACCCTCTGGCCGACG |
| | | CCCACCTGACCAAGAAGCTGCTGGACCTGGTGCAGCAGAGCTGCAA |
| | | CTACAAGCAGCTGAGAAAGGGCGCCAACGAGGCCACCAAGACCCTG |
| | | AACAGAGGCATCAGCGAGTTCATCGTGATGGCCGCCGACGCCGAGC |
| | | CTCTGGAGATCATCCTGCACCTGCCTCTGCTGTGCGAGGACAAGAAC |
| | | GTGCCTTACGTGTTCGTGAGAAGCAAGCAGGCCCTGGGCAGAGCCT |
| | | GCGGCGTGAGCAGACCTGTGATCGCCTGCAGCGTGACCATCAAGGA |
| | | GGGCAGCCAGCTGAAGCAGCAGATCCAGAGCATCCAGCAGAGCATC |
| | | GAGAGACTGCTGGTG |
| 73 | LARP7 (aa) | METESGNQEKVMEEESTEKKKEVEKKKRSRVKQVLADIAKQVDFWFG |
| | | DANLHKDRFLREQIEKSRDGYVDISLLVSFNKMKKLTTDGKLIARALRS |
| | | SAVVELDLEGTRIRRKKPLGERPKDEDERTVYVELLPKNVNHSWIERVF |
| | | GKCGNVVYISIPHYKSTGDPKGFAFVEFETKEQAAKAIEFLNNPPEEAPR |
| | | KPGIFPKTVKNKPIPALRVVEEKKKKKKKKGRMKKEDNIQAKEENMDT |
| | | SNTSISKMKRSRPTSEGSDIESTEPQKQCSKKKKKRDRVEASSLPEVRTG |
| | | KRKRSSSEDAESLAPRSKVKKIIQKDIIKEASEASKENRDIEISTEEEKDT |
| | | GDLKDSSLLKTKRKHKKKHKERHKMGEEVIPLRVLSKSEWMDLKKEY |
| | | LALQKASMASLKKTISQIKSESEMETDSGVPQNTGMKNEKTANREECR |
| | | TQEKVNATGPQFVSGVIVKIISTEPLPGRKQVRDTLAAISEVLYVDLLEG |
| | | DTECHARFKTPEDAQAVINAYTEINKKHCWKLEILSGDHEQRYWQKIL |
| | | VDRQAKLNQPREKKRGTEKLITKAEKIRLAKTQQASKHIRFSEYD |
| 74 | LARP7 (nt) | ATGGAGACCGAGAGCGGCAACCAGGAGAAGGTGATGGAGGAGGAG |
| | | AGCACCGAGAAGAAGAAGGAGGTGGAGAAGAAGAAGAGAAGCAG |
| | | AGTGAAGCAGGTGCTGGCCGACATCGCCAAGCAGGTGGACTTCTGG |
| | | TTCGGCGACGCCAACCTGCACAAGGACAGATTCCTGAGAGAGCAGA |
| | | TCGAGAAGAGCAGAGACGGCTACGTGGACATCAGCCTGCTGGTGAG |
| | | CTTCAACAAGATGAAGAAGCTGACCACCGACGGCAAGCTGATCGCC |
| | | AGAGCCCTGAGAAGCAGCGCCGTGGTGGAGCTGGACCTGGAGGGCA |
| | | CCAGAATCAGAAGAAAGAAGCCTCTGGGCGAGAGACCTAAGGACG |
| | | AGGACGAGAGAACCGTGTACGTGGAGCTGCTGCCTAAGAACGTGAA |
| | | CCACAGCTGGATCGAGAGAGTGTTCGGCAAGTGCGGCAACGTGGTG |
| | | TACATCAGCATCCCTCACTACAAGAGCACCGGCGACCCTAAGGGCTT |
| | | CGCCTTCGTGGAGTTCGAGACCAAGGAGCAGGCCGCCAAGGCCATC |
| | | GAGTTCCTGAACAACCCTCCTGAGGAGGCCCCTAGAAAGCCTGGCA |
| | | TCTTCCCTAAGACCGTGAAGAACAAGCCTATCCCTGCCCTGAGAGTG |
| | | GTGGAGGAGAAGAAGAAGAAGAAGAAGAAGAAGGGCAGAATGAA |
| | | GAAGGAGGACAACATCCAGGCCAAGGAGGAGAACATGGACACCAG |
| | | CAACACCAGCATCAGCAAGATGAAGAGAAGCAGACCTACCAGCGA |
| | | GGGCAGCGACATCGAGAGCACCGAGCCTCAGAAGCAGTGCAGCAA |
| | | GAAGAAGAAGAAGAGAGACAGAGTGGAGGCCAGCAGCCTGCCTGA |
| | | GGTGAGAACCGGCAAGAGAAAGAGAAGCAGCAGCGAGGACGCCGA |
| | | GAGCCTGGCCCCTAGAAGCAAGGTGAAGAAGATCATCCAGAAGGAC |
| | | ATCATCAAGGAGGCCAGCGAGGCCAGCAAGGAGAACAGAGACATC |
| | | GAGATCAGCACCGAGGAGGAGAAGGACACCGGCGACCTGAAGGAC |
| | | AGCAGCCTGCTGAAGACCAAGAGAAAGCACAAGAAGAAGCACAAG |
| | | GAGAGACACAAGATGGGCGAGGAGGTGATCCCTCTGAGAGTGCTGA |
| | | GCAAGAGCGAGTGGATGGACCTGAAGAAGGAGTACCTGGCCCTGCA |
| | | GAAGGCCAGCATGGCCAGCCTGAAGAAGACCATCAGCCAGATCAAG |
| | | AGCGAGAGCGAGATGGAGACCGACAGCGGCGTGCCTCAGAACACC |
| | | GGCATGAAGAACGAGAAGACCGCCAACAGAGAGGAGTGCAGAACC |
| | | CAGGAGAAGGTGAACGCCACCGGCCCTCAGTTCGTGAGCGGCGTGA |
| | | TCGTGAAGATCATCAGCACCGAGCCTCTGCCTGGCAGAAAGCAGGT |
| | | GAGAGACACCCTGGCCGCCATCAGCGAGGTGCTGTACGTGGACCTG |
| | | CTGGAGGGCGACACCGAGTGCCACGCCAGATTCAAGACCCCTGAGG |
| | | ACGCCCAGGCCGTGATCAACGCCTACACCGAGATCAACAAGAAGCA |
| | | CTGCTGGAAGCTGGAGATCCTGAGCGGCGACCACGAGCAGAGATAC |
| | | TGGCAGAAGATCCTGGTGGACAGACAGGCCAAGCTGAACCAGCCTA |
| | | GAGAGAAGAAGAGAGGCACCGAGAAGCTGATCACCAAGGCCGAGA |
| | | AGATCAGACTGGCCAAGACCCAGCAGGCCAGCAAGCACATCAGATT |
| | | CAGCGAGTACGAC |
| 55 | L7Ae | MYVRFEVPEDMQNEALSLLEKVRESGKVKKGTNETTKAVERGLAKLV |
| | | YIAEDVDPPEIVAHLPLLCEEKNVPYIYVKSKNDLGRAVGIEVPCASAAI |
| | | INEGELRKELGSLVEKIKGLQK |
| 151 | L7Ae (nt) | ATGTACGTGCGGTTCGAGGTGCCCGAGGACATGCAGAACGAGGCCCTGAGC |
| | | CTGCTGGAGAAGGTGCGGGAGAGCGGCAAGGTGAAGAAGGGCACCAACGA |
| | | GACCACCAAGGCCGTGGAGCGGGGTCTGGCTAAGCTGGTGTACATCGCCGA |
| | | GGACGTGGACCCTCCCGAGATCGTGGCCCACCTGCCCCTGCTGTGCGAGGA |
| | | GAAGAACGTGCCCTACATCTACGTGAAGAGTAAGAACGACCTTGGCCGGGC |
| | | CGTGGGCATCGAGGTCCCCTGCGCCAGCGCTGCCATCATCAACGAGGGCGA |
| | | GCTGCGGAAGGAGCTGGGCAGCCTGGTGGAGAAGATCAAGGGCCTGCAGA |
| | | AG |
| 71 | Snu13 | MTEADVNPKAYPLADAHLTKKLLDLVQQSCNYKQLRKGANEATKTLN |
| | | RGISEFIVMAADAEPLEIILHLPLLCEDKNVPYVFVRSKQALGRACGVSR |
| | | PVIACSVTIKEGSQLKQQIQSIQQSIERLLV |
| 72 | Snu13 (nt) | ATGACCGAGGCCGACGTGAACCCTAAGGCCTACCCTCTGGCCGACGCCCAC |
| | | CTGACCAAGAAGCTGCTGGACCTGGTGCAGCAGAGCTGCAACTACAAGCAG |
| | | CTGAGAAAGGGCGCCAACGAGGCCACCAAGACCCTGAACAGAGGCATCAG |
| | | CGAGTTCATCGTGATGGCCGCCGACGCCGAGCCTCTGGAGATCATCCTGCA |
| | | CCTGCCTCTGCTGTGCGAGGACAAGAACGTGCCTTACGTGTTCGTGAGAAG |
| | | CAAGCAGGCCCTGGGCAGAGCCTGCGGCGTGAGCAGACCTGTGATCGCCTG |
| | | CAGCGTGACCATCAAGGAGGGCAGCCAGCTGAAGCAGCAGATCCAGAGCA |
| | | TCCAGCAGAGCATCGAGAGACTGCTGGTG |

### Destabilization Domain

The fusion polypeptide of the timer systems of the disclosure features a destabilization domain. A “destabilization domain”, “destabilization tag”, or “degron” targets proteins for degradation and allows for the precise regulation of protein expression profiles in mammalian cells by modulating target protein half-lives. A degron is a myriad of small modifications that target proteins for proteolysis have been described and reviewed. See, e.g., Chassin, H., et al, Nat Commun 10, 2013 (2019). One example of a degron is ubiquitin (Ub), a small, highly conserved 76-amino-acid polypeptide modification that is covalently attached to proteins by the cooperative action of three enzymes, Ub-activating enzyme (E1), Ub-conjugating enzyme (E2), and Ub ligase (E3), which are components of the Ub-proteasome system. By linking Ub to intracellular proteins for targeting to the proteasome, cells can directly modulate the intracellular protein concentration.

Two major pathways target the proteins for degradation either via the N-end rule pathway or the Ub-fusion degradation (UFD) pathway. The N-end rule implies that the N-terminal amino acid of a protein determines its metabolic in vivo half-life. 3×UbVR, 3×UbVV, 2×UbVR, 2×UbVV, UbAR, UbVV, UbVR, UbAV, 2×UbAR, 2×UbAV target proteins to the UFD pathway. In these cases, the Gly76 residue of Ub is mutated and the C-terminal isopeptidase site is no longer recognized by deubiquitinating enzymes.

In some embodiments, the destabilization domain has a ubiquitin tag. The ubiquitin tag can be, for example, 3XUbVR, 3XUbVV, UbR, UbK, PEST, UbD, or UbM. Ubiquitin tags such as UbR, UbP, UbW, UbH, UbI, UbK, UbQ, UbV, UbL, UbD, UbN, UbG, UbY, UbT, UbS, UbF, UbA, UbC, UbE, UbM) with an intact C-terminal isopeptidase site can also be used in the present disclosure.

In some embodiments, the degradation tag is lacS, PEST, 2×PEST, or PESTmod. These degrons influence the half-lives of proteins either via the Ub-proteasome pathway, as described for the Escherichia coli Lac repressor-derived spacer30, or via a Ub-independent pathway as described in Chassin, H., et al, Nat Commun 10, 2013 (2019).

In some embodiments, varying the ratio of the timer (i.e., the repressor and/or fusion polypeptide) to the target polypeptide controls the onset of expression of the target RNA. In some embodiments, the ratio of repressor (or timer) to target polypeptide is any of the following: 1:0.5, 1:1, 1:2, 1:4, 1:5, 1:10.

### Repressor Binding Element

In some embodiments of the timer systems disclosed herein, the first polynucleotide of has a repressor binding element. A repressor binding element comprises a sequence, e.g., a DNA or RNA sequence, which is bound, e.g., recognized by a repressor (or timer) described elsewhere in this disclosure. In some embodiments, the repressor binds to a sequence comprising the binding element, or a fragment thereof.

In some embodiments, the repressor binds to a structure comprising the binding element, or a fragment thereof.

In some embodiments, the composition or system comprises a repressor or a fusion protein comprising a repressor that binds to e.g., recognizes, the repressor binding element of the first polynucleotide.

In some embodiments, the repressor binding element of the first polynucleotide is situated upstream (5′) or downstream (3′), or in the open reading frame of the sequence encoding the polypeptide.

In some embodiments, the repressor binding element of the first polynucleotide is situated upstream (5′) or downstream (3′) of a 5′ UTR of the first polynucleotide. In some embodiments, the binding element of the first polynucleotide is situated upstream (5′) or downstream (3′) of a 3′ UTR of the first polynucleotide. In some embodiments, the repressor binding element of the first polynucleotide is situated in the 5′ UTR of the first polynucleotide. In some embodiments, the repressor binding element of the first polynucleotide is situated downstream of a 3′ UTR of the first polynucleotide. In some embodiments, the repressor binding element of the first polynucleotide is situated adjacent, e.g., next to, a Poly A tail.

In some embodiments, the repressor binding element is MS2. In some embodiments, the repressor binding element is PP7. In some embodiments, the repressor binding element is BoxB. In some embodiments, the repressor binding element is U1A hairpin. In some embodiments, the repressor binding element is PRE. In some embodiments, the repressor binding element is PRE2. In some embodiments, the repressor binding element is a kink-turn forming sequence. In some embodiments, the repressor binding element is 7SK. In some embodiments, the repressor binding element is an RNA sequence/structure element that binds to a protein.

In some embodiments, when the binding element is MS2 (e.g., wildtype MS2, or a variant or fragment thereof) the repressor is MBP (e.g., wildtype MBP, a variant or fragment thereof).

In some embodiments, when the repressor binding element is PP7 (e.g., wildtype PP7, or a variant or fragment thereof) the repressor is PCP (e.g., wildtype PCP, or a variant or fragment thereof). PP7 can comprise the sequence of any one of the PP7 and variants thereof described in Lim F, and Peabody D S. Nucleic Acids Res. 2002; 30(19):4138-4144, and U.S. Pat. No. 9,365,831, incorporated by reference herein in its entirety.

In some embodiments, when the repressor binding element is BoxB (e.g., wildtype BoxB, or a variant or fragment thereof) the repressor is Lambda N (e.g., wildtype Lambda N, or a variant or fragment thereof).

In some embodiments, when the repressor binding element is U1A hairpin (e.g., wildtype U1A hairpin, or a variant or fragment thereof) the repressor is U1A (e.g., wildtype U1A, or a variant or fragment thereof).

In some embodiments, when the repressor binding element is PRE (e.g., wildtype PRE, or a variant or fragment thereof) the repressor is PUF (e.g., wildtype PUF, or a variant or fragment thereof).

In some embodiments, when the repressor binding element is a kink-turn forming sequence the repressor is 15.5 kd (e.g., wildtype 15.5 kd, or a variant or fragment thereof).

In some embodiments, when the repressor binding element is a 7SK sequence repressor is LARP7 (e.g., wildtype LARP7, or a variant or fragment thereof).

In some embodiments, the repressor binding element comprises a sequence comprising 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the repressor binding element comprises a sequence comprising about 5-100, about 5-90, about 5-80, about 5-70, about 5-60, about 5-50, about 5-40, about 5-30, about 5-25, about 5-20, about 5-19, about 5-18, about 5-17, about 5-16, about 5-15, about 5-14, about 5-13, about 5-12, about 5-11, about 5-10, about 5-9, about 5-8, about 5-7 or about 5-6 nucleotides. In some embodiments, the repressor binding element comprises a sequence comprising about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the repressor binding element comprises a sequence comprising about 5-100, about 6-90, about 7-80, about 8-70, about 9-60, about 10-50, about 11-40, about 12-30, about 13-25, about 14-24, about 15-23, about 16-22, about 17-21, or about 18-20 nucleotides. In some embodiments, the repressor binding element comprises a sequence comprising 19 nucleotides.

In some embodiments, the repressor binding element comprises a binding element nucleotide sequence provided in Table 4 or a sequence with at least 80%, 85%, 90%, 95%, 96%, 97%, 98%, 99% or 100% identity thereof. In some embodiments, the repressor binding element comprises a binding element sequence provided in SEQ ID NO: 1, SEQ ID NO: 2, SEQ ID NO: 3, or a sequence with at least 80%, 85%, 90%, 95%, 96%, 97%, 98%, 99% or 100% identity thereof.

In some embodiments of any of the compositions, systems, methods or uses disclosed herein, the repressor binding element comprises at least 1, 2, 3, 4, 5, 6, 7, 8, 9, 10, 11, 12, 13, 14, 15, 20 or 30 repeats of the sequence bound by the second polypeptide.

In some embodiments, the binding element comprises no more than 80, 70, 60, 50, 40 or repeats of the sequence bound by the second polypeptide. In some embodiments, the repressor binding element comprises about 1-30, about 1-20, about 1-10, about 1-9, about 1-8, about 1-7, about 1-6, about 1-5, about 1-4, about 1-3, or about 1-2 repeats of the sequence bound by the second polypeptide. In some embodiments, the repressor binding element comprises about 1-30, about 2-30, about 3-30, about 4-30 about, 5-30 about, 6-30, about 7-30, about 8-30, about 9-30, about 10-30, about 11-30, about 12-30, about 13-30, about 14-30, about 15-30, or about 20-30 repeats of the sequence bound by the second polypeptide. In some embodiments, the repressor binding element comprises about 1-30, about 2-20, about 3-15, about 4-14, about 5-13, about 6-12, about 7-11, or about 8-10 repeats of the sequence bound by the second polypeptide. In some embodiments, the repressor binding element comprises 6 repeats of the sequence bound by the second polypeptide.

In some embodiments of any of the compositions, systems, methods or uses disclosed herein, each repeat is separated by a spacer sequence comprising 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the spacer sequence comprises about 1-100, about 1-90, about 1-80, about 1-70, about 1-60, about 1-50, about 1-40, about 1-30, about 1-25, about 1-20, about 1-19, about 1-18, about 1-17, about 1-16, about 1-15, about 1-14, about 1-13, about 1-12, about 1-11, about 1-10, about 1-9, about 1-8, about 1-7, about 1-6, about 1-5, about 1-4, about 1-3, or about 1-2 nucleotides. In some embodiments, the spacer sequence comprises about 1-100, about 2-100, about 3-100, about 4-100, about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the spacer sequence comprises about 1-100, about 2-90, about 3-80, about 4-70, about 5-60, about 6-50, about 7-40, about 8-40, about 9-30, about 10-25, about 11-24, about 12-23, about 13-22, about 14-21, about 15-20, about 16-19, about 17-18 nucleotides. In some embodiment, the spacer sequence comprises 20 nucleotides.

In some embodiments, the spacer sequence comprises a spacer sequence provided in Table 4 or a sequence with at least 80%, 85%, 90%, 95%, 96%, 97%, 98%, 99% or 100% identity thereof.

In some embodiments, the repressor binding element is an MS2 dimer which comprises monomers linked by a linker sequence. The linker sequence could be any peptide sequence known to link two protein sequences, including but not limited to those known in the art (See, e.g., Chen et al., Adv Drug Deliv Rev. (2013) Oct. 15; 65(10): 1357-1369).

TABLE 4
Exemplary sequences of a repressor binding element

| | | |
|---|---|---|
| SEQ | Sequence | |
| ID NO | information | Sequence |

3′UTR sequences

| | | |
|---|---|---|
| 1 | 3′v1.1_6xMS2 | TGATAATAGGCTGGAGCCTCGGTGGCCTAGCTTCTTGCCCCTTGG |
| | | GCCTCCCCCCAGCCCCTCCTCCCCTTCCTGCACCCGTACCCCCGT |
| | | GGTCTTTGAATAAAGTCTGAGTGGGCGGCCAGGTATGATTACAA |
| | | CCTGTGCTACTTTAGATGACTATTGAAAGACCATTAGGCTTTCTA |
| | | ACGGACGCTCACGAGAGAGGGGCACCAATAGGGCCCCACGGCA |
| | | CTTCAAAGGTTTGGCTTGGAGTAGTAACCCAAGCAGCAACAGTT |
| | | TTGACTTTCGGACCACCATCAGGGGTCCCACGTTGGGAACACGT |
| | | AACTCTCCTACTAACAAGAGGAG |
| 2 | 3′6xMS2_3′v1.1 | TGATAATAGCAGGTATGATTACAACCTGTGCTACTTTAGATGACT |
| | | ATTGAAAGACCATTAGGCTTTCTAACGGACGCTCACGAGAGAGG |
| | | GGCACCAATAGGGCCCCACGGCACTTCAAAGGTTTGGCTTGGAG |
| | | TAGTAACCCAAGCAGCAACAGTTTTGACTTTCGGACCACCATCA |
| | | GGGGTCCCACGTTGGGAACACGTAACTCTCCTACTAACAAGAGG |
| | | AGGCTGGAGCCTCGGTGGCCTAGCTTCTTGCCCCTTGGGCCTCCC |
| | | CCCAGCCCCTCCTCCCCTTCCTGCACCCGTACCCCCGTGGTCTTT |
| | | GAATAAAGTCTGAGTGGGCGGC |
| 3 | 3′_6xMS2 | TGATAATAGCAGGTATGATTACAACCTGTGCTACTTTAGATGA |
| | (MS2 sequences | CTATTGAAAGACCATTAGGCTTTCTAACGGACGCTCACGAGAG |
| | in bold and | AGGGGCACCAATAGGGCCCCACGGCACTTCAAAGGTTTGGCTT |
| | underline) | GGAGTAGTAACCCAAGCAGCAACAGTTTTGACTTTCGGACCAC |
| | | CATCAGGGGTCCCACGTTGGGAACACGTAACTCTCCTACTAAC |
| | | AAGAGGAG |
| 4 | 3′v1.1 | TGATAATAGGCTGGAGCCTCGGTGGCCTAGCTTCTTGCCCCTTGG |
| | | GCCTCCCCCCAGCCCCTCCTCCCCTTCCTGCACCCGTACCCCCGT |
| | | GGTCTTTGAATAAAGTCTGAGTGGGCGGC |
| 5 | 3′v1.1_6xMS2 | TGATAATAGGCTGGAGCCTCGGTGGCCTAGCTTCTTGCCCCTTGG |
| | | GCCTCCCCCCAGCCCCTCCTCCCCTTCCTGCACCCGTACCCCCGT |
| | | GGTCTTTGAATAAAGTCTGAGTGGGCGGCCAGGTATGATTACAA |
| | | CCTGTGCTACTTTAGATGACTATTGAAAGACCATTAGGCTTTCTA |
| | | ACGGACGCTCACGAGAGAGGGGCACCAATAGGGCCCCACGGCA |
| | | CTTCAAAGGTTTGGCTTGGAGTAGTAACCCAAGCAGCAACAGTT |
| | | TTGACTTTCGGACCACCATCAGGGGTCCCACGTTGGGAACACGT |
| | | AACTCTCCTACTAACAAGAGGAG |

5′ UTR sequence*

| | | |
|---|---|---|
| 6 | 5′ v1.1 | GGGAAATAAGAGAGAAAAGAAGAGTAAGAAGAAATATAAGACC |
| | | CCGGCGCCGCCACC |
| 7 | 5′kink turn (5′kt) | GGAUCCGUGAUCGGAAACGUGAGAUCCACCUCAGAUCCGCUAG |
| | | GACACCCGCAGAUCGAGAAGAAGGCGAAUUAAGAGAGAAAAG |
| | | AAGAGUA AGAAGAAAUAUAAGACACCGGUCGC CACC |
| 8 | 5′ 6xMS2 | GGGAAACGGCAAGCAGCAGCUGCCGCUGUUUUGUCUUAACCGG |
| | (underlined | UUGAAAGACCAUUAGGCUUUCUAACGGACGCUCACGAGAGAG |
| | sequence indicates | GGGCACCAAUAGGGCCCCACGGCACUUCAAAGGUUUGGCUUGG |
| | the region MBP | AGUAGUAACCCAAGCAGCAACAGUUUUGACUUUCGGACCACCA |
| | binds to) | UCAGGGGUCCCACGUUGGGAACACGUAACUCUCCUACUAACAA |
| | | GAGGAGCCCCGGCGCCGCCACC |
| 9 | PRE_p2 | GGGAAAUAAGAGAGAAAAAGAGAGAGGGAGAGGAGAAGAGUA |
| | | AGAAGAAAUAUAAGACCCCGGCGCCGCCACC |
| 10 | PRE_p3 | GGGAAAUAAGAGAGAAAAGAAGAGUAAGAAAGAGAGAGGGAG |
| | | AGGAGAAAUAUAAGACCCCGGCGCCGCCACC |
| 11 | PRE_p4 | GGGAAAUAAGAGAGAAAAGAAGAGUAAGAAGAAAUAUAAGAC |
| | | AGAGAGAGGGAGAGGACCCGGCGCCGCCACC |
| 12 | PRE2_p1_3x | GGGAAAUUUUUUUUUGAUAUUAUAAGAGUUUUUUUUUGAUAU |
| | | UAAGAAAAUUUUUUUUUGAUAUUAGAAGAGUAAGAAGAAAUA |
| | | UAAGACCCCGGCGCCGCCACC |
| 13 | PRE2_p2_3x | GGGAAAUAAGAGUUUUUUUUUGAUAUUAAGAAAAUUUUUUUU |
| | | UGAUAUUAGAAGAGUUUUUUUUUGAUAUUAUAAGAAGAAAUA |
| | | UAAGACCCCGGCGCCGCCACC |
| 14 | PRE2_p3_3x | GGGAAAUAAGAGAGAAAAUUUUUUUUUGAUAUUAGAAGAGUU |
| | | UUUUUUUGAUAUUAUAAGAAUUUUUUUUUGAUAUUAGAAAUA |
| | | UAAGACCCCGGCGCCGCCACC |
| 15 | PRE2_p4_3x | GGGAAAUAAGAGAGAAAAGAAGAGUUUUUUUUUGAUAUUAUA |
| | | AGAAUUUUUUUUUGAUAUUAGAAAUAUUUUUUUUUGAUAUUA |
| | | UAAGACCCCGGCGCCGCCACC |
| 16 | PRE2_p5_3x | GGGAAAUAAGAGAGAAAAGAAGAGUAAGAAUUUUUUUUUGAU |
| | | AUUAGAAAUAUUUUUUUUUGAUAUUAUAAGACUUUUUUUUUG |
| | | AUAUUACCCGGCGCCGCCACC |
| 17 | PRE2_p6_3x | GGGAAAUAAGAGAGAAAAGAAGAGUAAGAAGAAAUAUUUUUU |
| | | UUUGAUAUUAUAAGACUUUUUUUUUGAUAUUACCCGGCUUUU |
| | | UUUUUGAUAUUAGCCGCCACC |

Miscellaneous sequences

| | | |
|---|---|---|
| 18 | BoxB | GGGCCCUGAAGAAGGGCCC |
| 19 | U1A hairpin | AAUCCAUUGCACUCCGGAUUU |
| 20 | 7SK | GGATGTGTGGCCAGTTGGTGCTGTCTGTCTTCTTGGTGACCAGGA |
| | | GTAATTTGGACCATCTGGGAAACCAGACCTCTTTTCACCCAGGCT |
| | | GTTTCGTGTGCATCACTTCTGAGGCTGTGTGCTCAGTCAAGACCT |
| | | TGCCAGAGAGTTGGGGGATTAGCCTTAGGTCGAGAATATTCTGA |
| | | GCAGTAGTAAGAAATTTAAAAATCATCCATAATTTCATCACTCTT |
| | | ATTTTAAGAGATTAGTTTAT |

*underlined sequence indicates the region the RBP binds to

### Modifications of Polynucleotides

In some embodiments of the timer systems disclosed herein, the design of the repressor-encoding polynucleotides can be modified in order to alter the half-life or stability of the repressor, thereby affecting the timing of target mRNA expression. For instance, a polynucleotide design modification described herein can reduce the half-life of a repressor, thereby reducing the degree of repression and advancing the timing of expression of the target mRNA. In another instance, a polynucleotide design modification described herein can increase the half-life of a repressor, thereby increasing the degree of repression and delaying the timing of expression of the target mRNA.

In some embodiments, the polynucleotide of the system (e.g., an mRNA) has one or more of the following design modifications: (1) an AU-rich element; (2) structurally accessible UTRs; and (3) a short polyA tail. The 3′ UTR comprises an AU-rich element, which can be 60%-90% AU-rich, for example, about 70% AU-rich. In some embodiments, the polyA tail of any of the polynucleotides described herein is 40-100 nucleotides in length.

### Methods of Using the Systems or Compositions

The present disclosure provides compositions, which can be delivered to cells, e.g., target cells, e.g., in vitro or in vivo. For in vitro protein expression, the cell is contacted with the composition by incubating the composition and the cell ex vivo. Such cells may subsequently be introduced in vivo. For in vivo protein expression, the cell is contacted with the composition by administering the composition to a subject to thereby induce protein expression in or on the desired cells within the subject. For example, in one embodiment, the composition is administered intravenously. In another embodiment, the composition is administered intramuscularly. In yet other embodiment, the composition is administered by a route selected from the group consisting of subcutaneously, intranodally and intratumorally.

For in vitro delivery, in one embodiment the cell is contacted with the composition by incubating the composition and the target cell ex vivo. In one embodiment, the cell is a human cell. Various types of cells have been demonstrated to be transfectable by the composition (e.g., the LNP).

In another embodiment, the cell is contacted with the composition for, e.g., at least 30 minutes, at least 1 hour, at least 2 hours, at least 3 hours, at least 4 hours, at least hours, at least 6 hours, at least 12 hours or at least 24 hours.

In one embodiment, the cell is contacted with the composition for a single treatment/transfection. In another embodiment, the cell is contacted with the composition for multiple treatments/transfections (e.g., two, three, four or more treatments/transfections of the same cells).

In another embodiment, for in vivo delivery, the cell is contacted with the composition by administering the composition to a subject to thereby deliver the polynucleotide(s) to cells within the subject. For example, in one embodiment, the composition is administered intravenously. In another embodiment, the composition is administered intramuscularly. In yet other embodiment, the composition is administered by a route selected from the group consisting of subcutaneously, intranodally and intratumorally.

In an aspect, provided herein is a method of expressing a polypeptide in a cell, comprising administering to the cell a composition disclosed herein.

In a related aspect, provided herein is a composition or system for use in a method of expressing a polypeptide in a cell.

In another aspect, the disclosure provides a method of expressing a polypeptide in a cell in a subject, comprising administering to the subject an effective amount of a composition disclosed herein.

In a related aspect, provided herein is a composition or system for use in a method of expressing a polypeptide in a cell in a subject.

In yet another aspect, provided herein is a method of delivering a composition disclosed herein.

In a related aspect, provided herein is a composition or system for use in a method of delivering the composition to a cell.

The cell can be any cell, including, but not limited to, a liver cell, a muscle cell, an immune cell, or a neuron.

In an embodiment, the method or use, comprises contacting the cell in vitro, in vivo or ex vivo with the composition or system.

In an embodiment, the composition or system formulated as an LNP, a liposome composition, a lipoplex composition, or a polyplex composition of the present disclosure is contacted with cells, e.g., ex vivo or in vivo and can be used to deliver a secreted polypeptide, an intracellular polypeptide, a transmembrane polypeptide, or peptides, polypeptides or biologically active fragments thereof to a subject.

In an aspect, the disclosure provides a method of delivering a composition or system disclosed herein to a subject having a disease or disorder, e.g., as described herein.

In a related aspect, provided herein is a composition or system for use in a method of delivering the composition or system to a subject having a disease or disorder, e.g., as described herein.

In another aspect, provided herein is a method of modulating an immune response in a subject, comprising administering to the subject in need thereof an effective amount of a composition or system disclosed herein.

In a related aspect, provided herein is a composition or system for use in a method of modulating an immune response in a subject, comprising administering to the subject an effective amount of the composition or system.

In another aspect, provided herein is a method of delivering a secreted polypeptide, an intracellular polypeptide, a transmembrane polypeptide, or peptides, polypeptides or biologically active fragments thereof to a subject.

In an aspect, provided herein is a method of treating, preventing, or preventing a symptom of, a disease or disorder comprising administering to a subject in need thereof an effective amount of a composition or system disclosed herein.

In a related aspect, provided herein is a composition or system for use in a method of treating, preventing, or preventing a symptom of, a disease or disorder in a subject, comprising administering to the subject in need thereof an effective amount of the composition or system.

In an embodiment, the first polynucleotide and/or the second polynucleotide of the system is formulated as an LNP. In an embodiment, the first polynucleotide of the system is formulated as an LNP. In an embodiment, the second polynucleotide of the system is formulated as an LNP. In an embodiment, both the first and the second polynucleotides of the system are formulated as LNPs.

In an embodiment, the LNP comprising the first polynucleotide is the same as the LNP comprising the second polynucleotide. In an embodiment, the LNP comprising the first polynucleotide is different from the LNP comprising the second polynucleotide.

In an embodiment, the LNP comprising the first polynucleotide is in a composition. In an embodiment, the LNP comprising the second polynucleotide is in a separate composition. In an embodiment, the LNP comprising the first polynucleotide and the LNP comprising the second polynucleotide are in the same composition. In some embodiments, the first and second polynucleotides are in separate dosage forms packaged together. In some embodiments, the first and second polynucleotides are in a unit dosage form.

In an embodiment, the LNP comprising the first polynucleotide and the LNP comprising the second polynucleotide are administered simultaneously, e.g., substantially simultaneously. In some embodiments, the LNP comprising the first polynucleotide and the LNP comprising the second polynucleotide are co-delivered.

In an embodiment, the LNP comprising the first polynucleotide and the LNP comprising the second polynucleotide are administered sequentially.

In an embodiment, the LNP comprising the first polynucleotide is administered first.

In an embodiment, the LNP comprising the first polynucleotide is administered first followed by administration of the LNP comprising the second polynucleotide.

In an embodiment, the LNP comprising the second polynucleotide is administered first.

In an embodiment, the LNP comprising the second polynucleotide is administered first followed by administration of the LNP comprising the first polynucleotide.

In some embodiments of the disclosed methods, the method comprises contacting the cell with a composition of the disclosure, wherein the composition comprises (a) a first polynucleotide comprising (i) a repressor binding element, and (ii) an open reading frame encoding a first polypeptide; and (b) a second polynucleotide comprising a sequence encoding a fusion polypeptide comprising (i) a repressor that binds to the repressor binding element, and (ii) a destabilization domain, wherein binding of the repressor to the repressor binding element reduces translation of the first polypeptide from the first polynucleotide, and wherein the destabilization domain enhances degradation of the repressor.

In some embodiments of the disclosed methods, the method comprises contacting the cell with a composition of the disclosure wherein the disclosure comprises (a) a first polynucleotide comprising (i) a repressor binding element, and (ii) an open reading frame encoding a first polypeptide; (b) a second polynucleotide comprising an open reading frame encoding a second polypeptide; and (c) a third polynucleotide comprising a sequence encoding a fusion polypeptide comprising (i) a repressor that binds to the repressor binding element, and (ii) a destabilization domain; and wherein binding of the repressor to the repressor binding element reduces translation of the first polypeptide from the first polynucleotide, and wherein the destabilization domain enhances degradation of the repressor.

In some embodiments of the disclosed methods, the method comprises contacting the cell with a composition of the disclosure wherein the disclosure comprises (a) a first polynucleotide comprising (i) a first repressor binding element, and (ii) an open reading frame encoding a first polypeptide; (b) a second polynucleotide comprising (i) a second repressor binding element, and (ii) an open reading frame encoding a second polypeptide; (c) a third polynucleotide comprising a sequence encoding a first fusion polypeptide comprising (i) a first repressor that binds to the first repressor binding element, and (ii) a first destabilization domain; and (d) a fourth polynucleotide comprising a sequence encoding a second fusion polypeptide comprising (i) a second repressor that binds to the second repressor binding element, and (ii) a second destabilization domain; and wherein binding of the first and second repressors to the first and second repressor binding elements respectively reduce translation of the first and second polypeptides from the first and second polynucleotides, and wherein the first and second destabilization domains respectively degrade the first and second repressors at different rates.

In some embodiments of the disclosed methods, the method comprises contacting the cell with a composition of the disclosure wherein the disclosure comprises (a) a first polynucleotide comprising (i) a first repressor binding element, and (ii) an open reading frame encoding a first polypeptide; (b) a second polynucleotide comprising (i) a second repressor binding element, and (ii) an open reading frame encoding a second polypeptide; (c) a third polynucleotide comprising an open reading frame encoding a third polypeptide; (d) a fourth polynucleotide comprising a sequence encoding a first fusion polypeptide comprising (i) a first repressor that binds to the first repressor binding element, and (ii) a first destabilization domain; and (e) a fifth polynucleotide comprising a sequence encoding a second fusion polypeptide comprising (i) a second repressor that binds to the second repressor binding element, and (ii) a second destabilization domain; and wherein binding of the first and second repressors to the first and second repressor binding elements respectively reduce translation of the first and second polypeptides from the first and second polynucleotides, and wherein the first and second destabilization domains respectively degrade the first and second repressors at different rates.

The timer systems disclosed herein delay or stagger RNA expression and have several uses. In one example, staggered expression of the polynucleotides of the timer system can be used to ensure correct antibody pairing that ensures functional antibodies, e.g., a bispecific antibody in which each arm binds to a different antigen. In another example, staggered expression ensures proper virus-like particle (VLP) formation. In another example, delayed expression of RNA can delay payload delivery of immune-oncology targets, for instance in a cancer, expression of a payload can be delayed until monocytes traffic to a tumor. In another example, the timing of antigen presentation and type 1 interferon induction can be delayed to enhance CD8+ T cell response.

### Sequence Optimization and Methods Thereof

In some embodiments, a polynucleotide of the disclosure comprises a sequence-optimized nucleotide sequence encoding a polypeptide disclosed herein, e.g., a polynucleotide encoding a polypeptide (e.g., a therapeutic or prophylactic protein), or a repressor. In some embodiments, the polynucleotide of the disclosure comprises an open reading frame (ORF) encoding a polypeptide or a repressor, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics. In some embodiments, the percentage of uracil or thymine nucleobases in a sequence-optimized nucleotide sequence (e.g., encoding a polypeptide or a repressor, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response in desired cells and/or microenvironments when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine (T) is present instead of uracil (U), and one would substitute T where U appears. For RNA it is recognized that uracil (U) is present instead of thymine (T). One of skill in the art could readily obtain an RNA sequence when the DNA sequence is provided by substituting thymine in the DNA sequence to uracil. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding a polypeptide, or a repressor of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding a polypeptide, or a repressor of the disclosure has a reduced number of uracil triplets (UUU) with respect to the wild-type nucleic acid sequence. In some embodiments, a uracil-modified sequence encoding a polypeptide, or a repressor has a reduced number of uracil pairs (UU) with respect to the number of uracil pairs (UU) in the wild-type nucleic acid sequence. In some embodiments, a uracil-modified sequence encoding polypeptide, or a repressor of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence”, refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding a polypeptide, or a repressor has a % UUwt between below 100%.

In some embodiments, the polynucleotide of the disclosure comprises a uracil-modified sequence encoding an encoding a polypeptide, or a repressor disclosed herein. In some embodiments, the uracil-modified sequence encoding a polypeptide, or a repressor comprises at least one chemically modified nucleobase, e.g., 5-methoxyuracil. In some embodiments, at least 95% of a nucleobase (e.g., uracil) in a uracil-modified sequence encoding a polypeptide, or a repressor of the disclosure are modified nucleobases. In some embodiments, at least 95% of uracil in a uracil-modified sequence encoding a polypeptide, or a repressor is 5-methoxyuracil.

In some embodiments, a polynucleotide of the disclosure (e.g., a polynucleotide comprising a nucleotide sequence encoding a polypeptide, or a repressor (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding a polypeptide, or a repressor. Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., an mRNA) can be administered to a subject in need thereof to facilitate in vivo expression of functionally active encoding a polypeptide, or a repressor.

Additional and exemplary methods of sequence optimization are disclosed in International PCT application WO 2017/201325, filed on 18 May 2017, the entire contents of which are hereby incorporated by reference.

### IVT Polynucleotide Architecture

In some embodiments, the polynucleotide of the present disclosure comprising an mRNA encoding a polypeptide, or a repressor is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded polypeptide, or repressor. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding the polypeptide, or the repressor can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of the polypeptide, or the repressor or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### Regions Having a 5′ Cap

The disclosure also includes a polynucleotide that comprises both a 5′ Cap and a polynucleotide of the present disclosure (e.g., a polynucleotide comprising a nucleotide sequence encoding a polypeptide, or a repressor).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present disclosure (e.g., a polynucleotide comprising a nucleotide sequence encoding a first polypeptide, a polypeptide, or a repressor) incorporate a cap moiety.

In some embodiments, polynucleotides of the present disclosure (e.g., a polynucleotide comprising a nucleotide sequence encoding a polypeptide, or a repressor) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the disclosure (e.g., a polynucleotide comprising a nucleotide sequence encoding a polypeptide, or a repressor), can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the CapI structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly A Tails

In some embodiments, the polynucleotides of the present disclosure (e.g., a polynucleotide comprising a nucleotide sequence encoding a polypeptide, or a repressor) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO: 149).

(SEQ ID NO: 149)
aaaaaaaaaa aaaaaaaaaa aaaaaaaaaa aaaaaaaaaa aaaaaaaaaa aaaaaaaaaa aaaaaaaaaa
aaaaaaaaaa aaaaaaaaaa aaaaaaaaaa

PolyA tails can also be added after the construct is exported from the nucleus.

According to the present disclosure, terminal groups on the poly A tail can be incorporated for stabilization. Polynucleotides of the present disclosure can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present disclosure can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs (Norbury, Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present disclosure. Generally, the length of a poly-A tail, when present, is greater than nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about to about 3,000 nucleotides (e.g., from 30 to 50, from 30 to 100, from 30 to 250, from to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO: 150).

(SEQ ID NO: 150)
aaaaaaaaaa aaaaaaaaaa aaaaaaaaaa aaaaaaaaaa aaaaaaaaaa aaaaaaaaaa aaaaaaaaaa
aaaaaaaaaa aaaaaaaaaa aaaaaaaaaa aaaaaaaaaa aaaaaaaaaa

### Start Codon Region

The disclosure also includes a polynucleotide that comprises both a start codon region and the polynucleotide described herein (e.g., a polynucleotide comprising a nucleotide sequence encoding a polypeptide or a repressor). In some embodiments, the polynucleotides of the present disclosure can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The disclosure also includes a polynucleotide that comprises both a stop codon region and the polynucleotide described herein (e.g., a polynucleotide comprising a nucleotide sequence encoding a polypeptide or a repressor). In some embodiments, the polynucleotides of the present disclosure can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present disclosure include the stop codon TGA in the case or DNA, or the stop codon UGA in the case of RNA, and one additional stop codon. In a further embodiment the addition stop codon can be TAA or UAA. In another embodiment, the polynucleotides of the present disclosure include three consecutive stop codons, four stop codons, or more.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1ψ) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 10% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the systems, or compositions disclosed herein.

In some embodiments of the disclosure, the polynucleotide are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient.

The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

### Delivery Agents

The compositions and systems disclosed herein further comprises a delivery agent. The delivery agent of the present disclosure can include, without limitation, liposomes, lipid nanoparticles, lipidoids, polymers, lipoplexes, polyplexes, microvesicles, exosomes, peptides, proteins, cells transfected with polynucleotides, hyaluronidase, nanoparticle mimics, nanotubes, conjugates, and combinations thereof.

a. Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and one or more polynucleotides disclosed herein, e.g., mRNA, has an increased therapeutic index as compared to a corresponding formulation which comprises a reference lipid (e.g., MC3, KC2, or DLinDMA) and the same one or more polyucleotides.

### (I) Lipid Nanoparticle Compositions

In some embodiments, the nucleic acids of the disclosure are formulated as lipid nanoparticle (LNP) compositions. The LNPs disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below. Lipid nanoparticles typically comprise amino lipid, phospholipid, structural lipid and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entireties.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

(1)(a) Amino Lipids

In some aspects, the amino lipids of a lipid nanoparticle composition disclosed herein may be one or more of compounds of Formula (I):

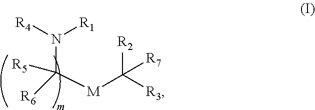

or their N-oxides, or salts or isomers thereof, wherein:
R1 is selected from the group consisting of C5-30 alkyl, C5-20 alkenyl, —R*YR″, —YR″, and —R″M′R′;
R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle;
R4 is selected from the group consisting of hydrogen, a C3-6 carbocycle, —(CH2)nQ, —(CH2)nCHQR,
—CHQR, —CQ(R)2, and unsubstituted C1-6 alkyl, where Q is selected from a carbocycle, heterocycle, —OR, —O(CH2)nN(R)2, —C(O)OR, —OC(O)R, —CX3, —CX2H, —CXH2, —CN, —N(R)2, —C(O)N(R)2, —N(R)C(O)R, —N(R)S(O)2R, —N(R)C(O)N(R)2, —N(R)C(S)N(R)2, —N(R)R8, —N(R)S(O)2R8, —O(CH2)nOR, —N(R)C(═NR9)N(R)2, —N(R)C(═CHR9)N(R)2, —OC(O)N(R)2,
—N(R)C(O)OR, —N(OR)C(O)R, —N(OR)S(O)2R, —N(OR)C(O)OR, —N(OR)C(O)N(R)2, —N(OR)C(S)N(R)2, —N(OR)C(═NR9)N(R)2, —N(OR)C(═CHR9)N(R)2, —C(═NR9)N(R)2, —C(═NR9)R, —C(O)N(R)OR, and —C(R)N(R)2C(O)OR, and each n is independently selected from 1, 2, 3, 4, and 5;
each R5 is independently selected from the group consisting of C1-3 alkyl, C2-3 alkenyl, and H;
each R6 is independently selected from the group consisting of C1-3 alkyl, C2-3 alkenyl, and H;
M and M′ are independently selected
from —C(O)O—, —OC(O)—, —OC(O)-M″-C(O)O—, —C(O)N(R′)—,
—N(R′)C(O)—, —C(O)—, —C(S)—, —C(S)S—, —SC(S)—, —CH(OH)—, —P(O)(OR′)O—, —S(O)2-, —S—S—, an aryl group, and a heteroaryl group, in which M″ is a bond, C1-13 alkyl or C2-13 alkenyl;
R7 is selected from the group consisting of C1-3 alkyl, C2-3 alkenyl, and H;
R8 is selected from the group consisting of C3-6 carbocycle and heterocycle;
R9 is selected from the group consisting of H, CN, NO2, C1-6 alkyl, —OR, —S(O)2R, —S(O)2N(R)2, C2-6 alkenyl, C3-6 carbocycle and heterocycle;
each R is independently selected from the group consisting of C1-3 alkyl, C2-3 alkenyl, and H;
each R′ is independently selected from the group consisting of C1-18 alkyl, C2-18 alkenyl, —R*YR″, —YR″, and H;
each R″ is independently selected from the group consisting of C3-15 alkyl and C3-15 alkenyl;
each R* is independently selected from the group consisting of C1-12 alkyl and C2-12 alkenyl;
each Y is independently a C3-6 carbocycle;
each X is independently selected from the group consisting of F, Cl, Br, and I; and m is selected from 5, 6, 7, 8, 9, 10, 11, 12, and 13; and wherein when R4 is —(CH2)nQ, —(CH2)nCHQR, —CHQR, or —CQ(R)2, then (i) Q is not —N(R)2 when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

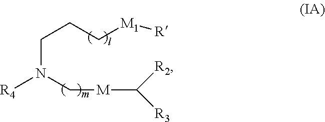

or its N-oxide, or a salt or isomer thereof, wherein 1 is selected from 1, 2, 3, 4, and 5; m is selected from 5, 6, 7, 8, and 9; M1 is a bond or M′; R4 is hydrogen, unsubstituted C1-3 alkyl, or —(CH2)nQ, in which Q is OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, m is 5, 7, or 9. For example, Q is OH, —NHC(S)N(R)2, or —NHC(O)N(R)2. For example, Q is —N(R)C(O)R, or —N(R)S(O)2R.

In certain embodiments, a subset of compounds of Formula (I) includes those of Formula (IB):

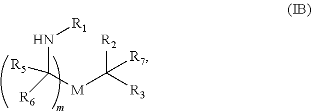

or its N-oxide, or a salt or isomer thereof in which all variables are as defined herein. For example, m is selected from 5, 6, 7, 8, and 9; R4 is hydrogen, unsubstituted C1-3 alkyl, or —(CH2)nQ, in which Q is OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, m is 5, 7, or 9. For example, Q is OH, —NHC(S)N(R)2, or —NHC(O)N(R)2. For example, Q is —N(R)C(O)R, or —N(R)S(O)2R.

In certain embodiments, a subset of compounds of Formula (I) includes those of Formula (II):

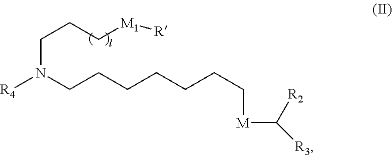

or its N-oxide, or a salt or isomer thereof, wherein 1 is selected from 1, 2, 3, 4, and 5; M1 is a bond or M′; R4 is hydrogen, unsubstituted C1-3 alkyl, or —(CH2)nQ, in which n is 2, 3, or 4, and Q is OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl.

In one embodiment, the compounds of Formula (I) are of Formula (IIa),

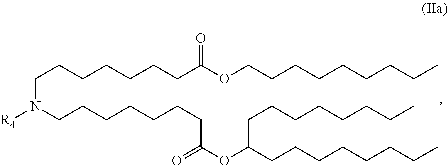

or their N-oxides, or salts or isomers thereof, wherein R4 is as described herein.

In another embodiment, the compounds of Formula (I) are of Formula (IIb),

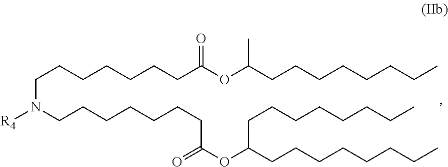

or their N-oxides, or salts or isomers thereof, wherein R4 is as described herein.

In another embodiment, the compounds of Formula (I) are of Formula (IIc) or (IIe):

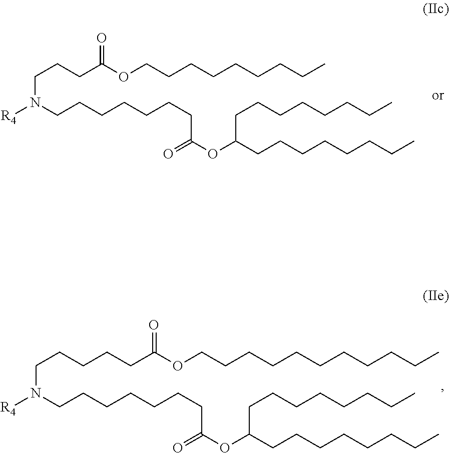

or their N-oxides, or salts or isomers thereof, wherein R4 is as described herein.

In another embodiment, the compounds of Formula (I) are of Formula (IIf).

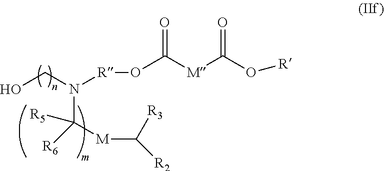

or their N-oxides, or salts or isomers thereof,
wherein M is —C(O)O— or —OC(O)—, M″ is C1-6 alkyl or C2-6 alkenyl, R2 and R3 are independently selected from the group consisting of C5-14 alkyl and C5-14 alkenyl, and n is selected from 2, 3, and 4.

In a further embodiment, the compounds of Formula (I) are of Formula (IId),

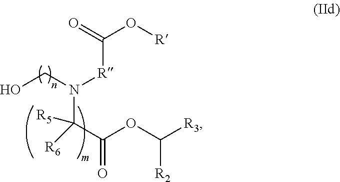

or their N-oxides, or salts or isomers thereof, wherein n is 2, 3, or 4; and m, R′, R″, and R2 through R6 are as described herein. For example, each of R2 and R3 may be independently selected from the group consisting of C5-14 alkyl and C5-14 alkenyl.

In a further embodiment, the compounds of Formula (I) are of Formula (IIg),

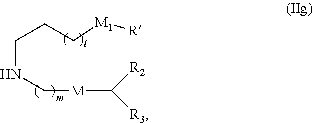

or their N-oxides, or salts or isomers thereof, wherein 1 is selected from 1, 2, 3, 4, and 5; m is selected from 5, 6, 7, 8, and 9; M1 is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, M″ is C1-6 alkyl (e.g., C1-4 alkyl) or C2-6 alkenyl (e.g. C2-4 alkenyl). For example, R2 and R3 are independently selected from the group consisting of C5-14 alkyl and C5-14 alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

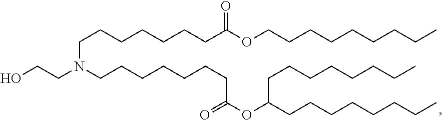 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

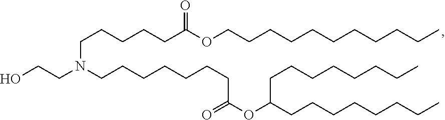 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

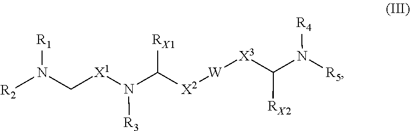

or salts or isomers thereof, wherein

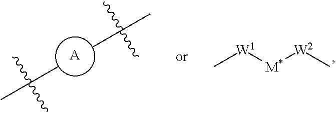

W is
ring A is

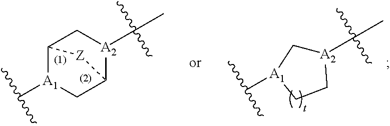

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —S C(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—,
   —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂-12 alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

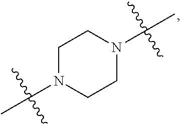 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (IIIa1)-(IIIa8):

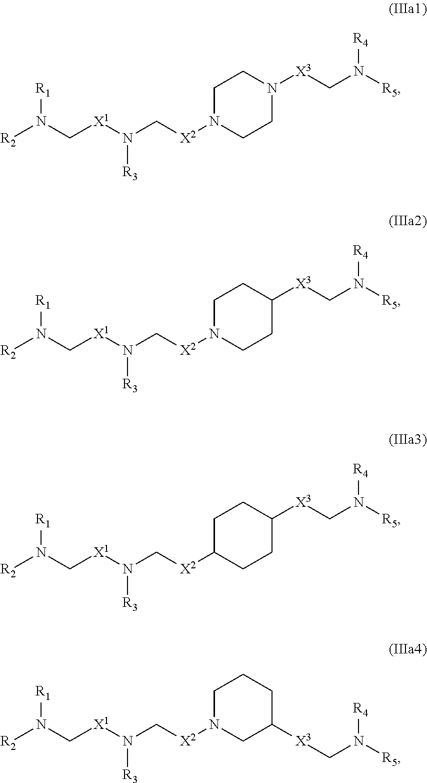

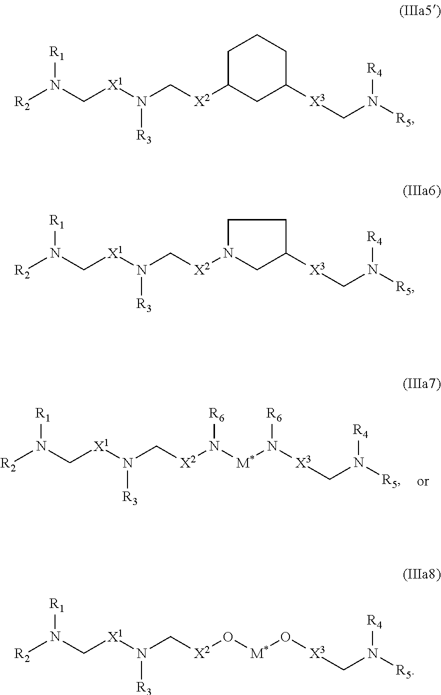

In some embodiments, the amino lipid is

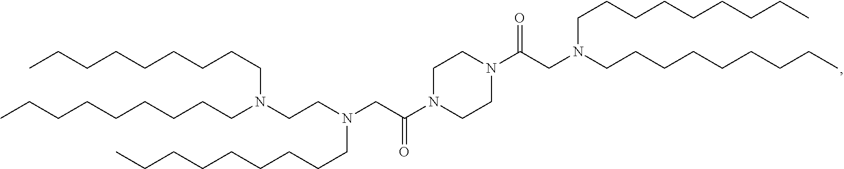 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH.

Thus, a lipid may have a positive or partial positive charge at physiological pH.

### (I)(b) Phospholipids

The lipid composition of a lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present disclosure is a compound of Formula (IV):

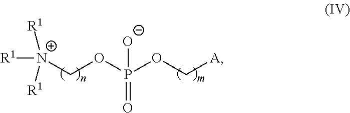

or a salt thereof, wherein:
each R1 is independently optionally substituted alkyl; or optionally two R1 are joined together with the intervening atoms to form optionally substituted monocyclic carbocyclyl or optionally substituted monocyclic heterocyclyl; or optionally three R1 are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
   A is of the formula:

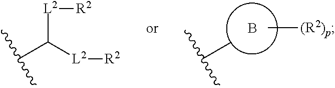

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), —OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

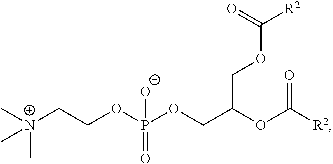

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530, or in International Application PCI/US2018/037922 filed on 15 Jun. 2018, the entire contents of each of which is hereby incorporated by reference in its entirety.

### i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

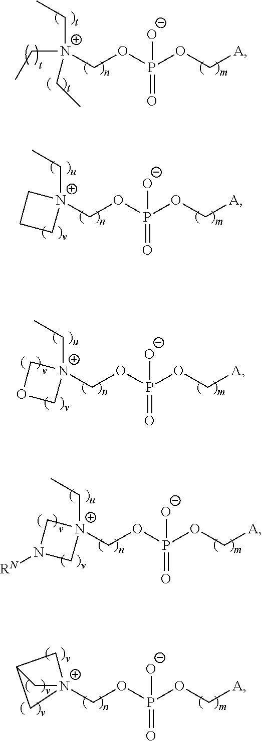

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

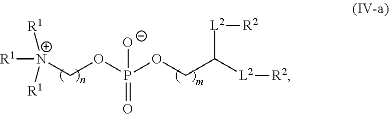

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

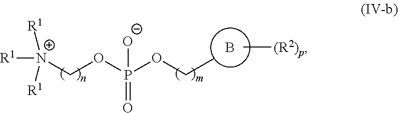

or a salt thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁-30 alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

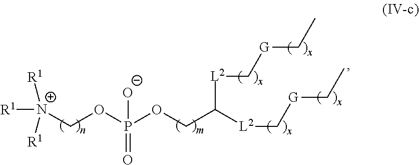

or a salt thereof, wherein:
each x is independently an integer between 0-30, inclusive; and each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O) NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}) NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

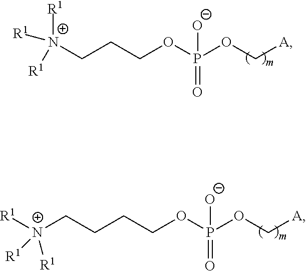

or a salt thereof.
   (iii) Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, an alternative lipid is useful.

In certain embodiments, an alternative lipid is used in place of a phospholipid of the present disclosure. In certain embodiments, an alternative lipid of the invention is oleic acid. In certain embodiments, the alternative lipid is one of the following:

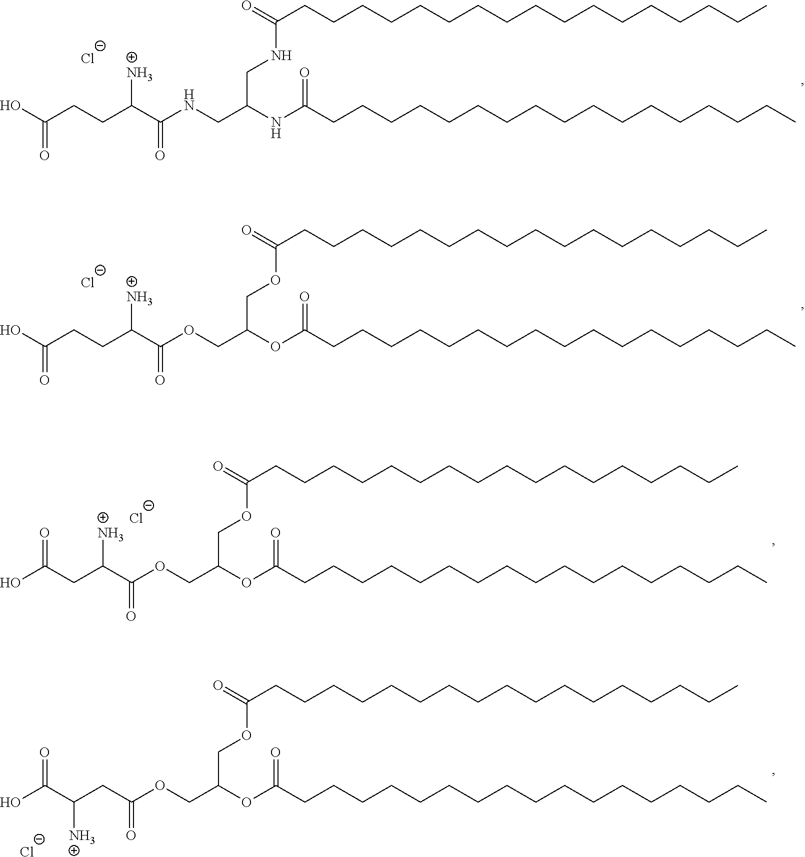

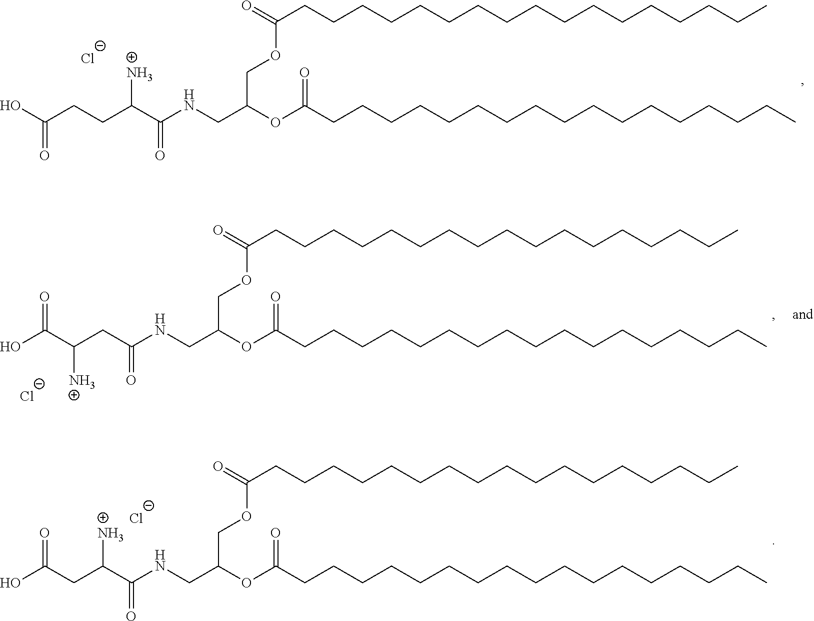 [SMILES: CCCCCCCCCCCCCCCCCC(=O)OCC(CNC(=O)C([NH3+])CCC(=O)O)OC(=O)CCCCCCCCCCCCCCCCC ; [Cl-] ; CCCCCCCCCCCCCCCCCC(=O)OCC(CNC(=O)CC([NH3+])C(=O)O)OC(=O)CCCCCCCCCCCCCCCCC ; [Cl-] ; CCCCCCCCCCCCCCCCCC(=O)OCC(CNC(=O)C([NH3+])CC(=O)O)OC(=O)CCCCCCCCCCCCCCCCC ; [Cl-]]

### (I)(c) Structural Lipids

The lipid composition of a lipid nanoparticle composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### (I)(d) Polyethylene Glycol (PEG)-Lipids

The lipid composition of a lipid nanoparticle composition disclosed herein disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C14 to about C22, preferably from about C14 to about C16. In some embodiments, a PEG moiety, for example an mPEG-NH2, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

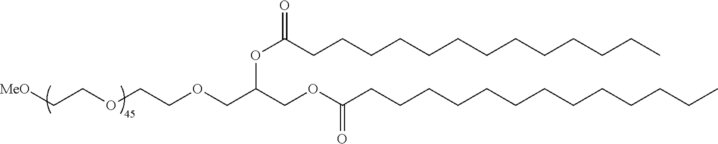

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

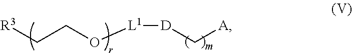

or salts thereof, wherein:
R3 is —ORO;
RO is hydrogen, optionally substituted alkyl, or an oxygen protecting group;
r is an integer between 1 and 100, inclusive;
L1 is optionally substituted C1-10 alkylene, wherein at least one methylene of the optionally substituted C1-10 alkylene is independently replaced with optionally substituted carbocyclylene, optionally substituted heterocyclylene, optionally substituted arylene, optionally substituted heteroarylene, O, N(RN), S, C(O), C(O)N(RN), —NRNC(O), C(O)O, OC(O), OC(O)O, OC(O)N(RN), NRNC(O)O, or NRNC(O)N(RN); D is a moiety obtained by click chemistry or a moiety cleavable under physiological conditions;
m is 0, 1, 2, 3, 4, 5, 6, 7, 8, 9, or 10;
A is of the formula:

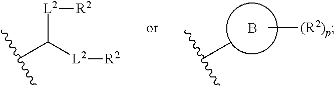

each instance of L2 is independently a bond or optionally substituted C₁₋₆ alkylene, wherein one methylene unit of the optionally substituted C₁₋₆ alkylene is optionally replaced with O, N(RN), S, C(O), C(O)N(RN), NRNC(O), C(O)O, OC(O), OC(O)O, —OC(O)N(RN), NRNC(O)O, or NRNC(O)N(RN);
each instance of R₂ is independently optionally substituted C₁₋₃₀ alkyl, optionally substituted C₁₋₃₀ alkenyl, or optionally substituted C₁₋₃₀ alkynyl; optionally wherein one or more methylene units of R₂ are independently replaced with optionally substituted carbocyclylene, optionally substituted heterocyclylene, optionally substituted arylene, optionally substituted heteroarylene, N(RN), O, S, C(O), C(O)N(RN), —NRNC(O), NRNC(O)N(RN), C(O)O, OC(O), OC(O)O, OC(O)N(RN), NRNC(O)O, —C(O)S, SC(O), C(═NRN), C(═NRN)N(RN), NRNC(═NRN), NRNC(═NRN)N(RN), —C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), —N(RN)S(O)O, S(O)2, N(RN)S(O)2, S(O)2N(RN), N(RN)S(O)2N(RN), OS(O)2N(RN), or N(RN)S(O)2O;
each instance of RN is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R3 is —ORO, and RO is hydrogen). In certain embodiments, the compound of Formula (V) is of Formula (V-OH):

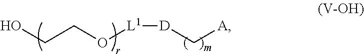

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI-A):

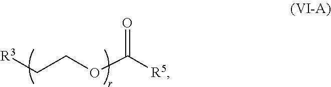

or a salts thereof, wherein:
R3 is —ORO;
RO is hydrogen, optionally substituted alkyl or an oxygen protecting group;
r is an integer between 1 and 100, inclusive;
R5 is optionally substituted C10-40 alkyl, optionally substituted C10-40 alkenyl, or optionally substituted C10-40 alkynyl; and optionally one or more methylene groups of R5 are replaced with optionally substituted carbocyclylene, optionally substituted heterocyclylene, optionally substituted arylene, optionally substituted heteroarylene, —N(RN), O, S, C(O), C(O)N(RN), NRNC(O), NRNC(O)N(RN), C(O)O, OC(O), OC(O)O, OC(O)N(RN), NRNC(O)O, C(O)S, SC(O), C(═NRN), C(═NRN)N(RN), NRNC(═NRN), NRNC(═NRN)N(RN), C(S), C(S)N(RN), NRNC(S), NRNC(S)N(RN), S(O), OS(O), —S(O)O, OS(O)O, OS(O)2, S(O)2O, OS(O)2O, N(RN)S(O), S(O)N(RN), —N(RN)S(O)N(RN), OS(O)N(RN), N(RN)S(O)O, S(O)2, N(RN)S(O)2, S(O)2N(RN), —N(RN)S(O)2N(RN), OS(O)2N(RN), or N(RN)S(O)2O; and each instance of RN is independently hydrogen, optionally substituted alkyl, or a nitrogen protecting group.

In certain embodiments, the compound of Formula (VI) is of Formula (VI-OH):

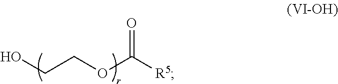

also referred to as (VI-B),
or a salt thereof. In some embodiments, r is 40-50.

In yet other embodiments the compound of Formula (VI-C) is:

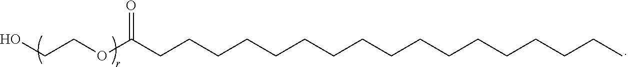

or a salt thereof.

In one embodiment, the compound of Formula (VI-D) is

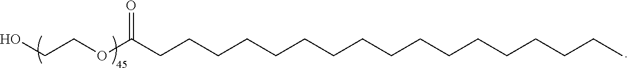

In some aspects, the lipid composition of the pharmaceutical compositions disclosed herein does not comprise a PEG-lipid. In some embodiments, the PEG-lipids may be one or more of the PEG lipids described in U.S. Application No. U.S. Ser. No. 15/674,872.

In some embodiments, a LNP of the disclosure comprises an amino lipid of any of Formula I, II or III, a phospholipid comprising DSPC, a structural lipid, and a PEG lipid comprising PEG-DMG. In some embodiments, a LNP of the disclosure comprises an amino lipid of any of Formula I, II or III, a phospholipid comprising DSPC, a structural lipid, and a PEG lipid comprising a compound having Formula VI. In some embodiments, a LNP of the disclosure comprises an amino lipid of Formula I, II or III, a phospholipid comprising a compound having Formula IV, a structural lipid, and the PEG lipid comprising a compound having Formula V or VI. In some embodiments, a LNP of the disclosure comprises an amino lipid of Formula I, II or III, a phospholipid comprising a compound having Formula IV, a structural lipid, and the PEG lipid comprising a compound having Formula V or VI. In some embodiments, a LNP of the disclosure comprises an amino lipid of Formula I, II or III, a phospholipid having Formula IV, a structural lipid, and a PEG lipid comprising a compound having Formula VI.

In some embodiments, a LNP of the disclosure comprises an N:P ratio of from about 2:1 to about 30:1. In some embodiments, a LNP of the disclosure comprises an N:P ratio of about 6:1. In some embodiments, a LNP of the disclosure comprises an N:P ratio of about 3:1, 4:1, or 5:1. In some embodiments, a LNP of the disclosure comprises a wt/wt ratio of the amino lipid component to the RNA of from about 10:1 to about 100:1. In some embodiments, a LNP of the disclosure comprises a wt/wt ratio of the amino lipid component to the RNA of about 20:1. In some embodiments, a LNP of the disclosure comprises a wt/wt ratio of the amino lipid component to the RNA of about 10:1.

In some embodiments, a LNP of the disclosure has a mean diameter from about nm to about 150 nm. In some embodiments, a LNP of the disclosure has a mean diameter from about 60 nm to about 120 nm.

b. Nanoparticle Compositions

In some embodiments, the pharmaceutical compositions disclosed herein are formulated as lipid nanoparticles (LNP). Accordingly, the present disclosure also provides nanoparticle compositions comprising (I) a lipid composition comprising a delivery agent such as compound as described herein, and (II) (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.

In some embodiments, the present disclosure also provides nanoparticle compositions comprising (I) a lipid composition comprising a delivery agent such as compound as described herein, and (II)(a) a first polynucleotide comprising (i) an open reading frame encoding a first polypeptide, (ii) an effector binding element, and (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element increases translation of the first polypeptide from the first polynucleotide.

In some embodiments, the present disclosure also provides nanoparticle compositions comprising (I) a lipid composition comprising a delivery agent such as compound as described herein, and (II) the polynucleotides of the timer system.

In such nanoparticle compositions described above, the lipid composition disclosed herein can encapsulate the polynucleotide encoding a first polypeptide and, when present, the polynucleotide encoding the second polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like.

The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, an ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a first polypeptide, and optionally in combination with the polynucleotide encoding a second polypeptide, are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 m or shorter (e.g., 1 m, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

c. Liposomes, Lipoplexes, and Lipid Nanoparticles

In some embodiments, the nucleic acids of the disclosure are formulated as liposome compositions, lipoplex compositions, and/or polyplex compositions. Such compositions, and methods are generally known in the art, see for example Itziar Gómez-Aguado I. et al., Nanomaterials 2020, 10, 364; Kowalski P. S. et al., Mol Ther. 2019 Apr. 10; 27(4): 710-728; ur Rehman Z, et al. ACS Nano. 2013 May 28; 7(5):3767-77; and U.S. Patent Application Publication US20160367702, incorporated by reference herein in its entirety.

In some embodiments, the compositions or formulations of the present disclosure comprise a delivery agent, e.g., a liposome, a lipoplexes, a lipid nanoparticle, or any combination thereof. The polynucleotides described herein (e.g., a polynucleotide comprising first polypeptide) can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MVLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the polynucleotides described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the polynucleotides described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the polynucleotides described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the polynucleotides described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (1 iE,20Z,23Z)—N,N-dimethylnonacosa-11,20,2-trien-10-amine, and any combination thereof. Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.
The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.
The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the polynucleotide described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Nati Acad Sci USA. 2007 6; 104:4095-4100; deFougerolles Hum Gene Ther. 2008 19:125-132; all of which are incorporated herein by reference in its entirety).

In some embodiments, the polynucleotides described herein are formulated as a solid lipid nanoparticle (SLN), which can be spherical with an average diameter between to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the polynucleotides described herein can be formulated for controlled release and/or targeted delivery. As used herein, “controlled release” refers to a pharmaceutical composition or compound release profile that conforms to a particular pattern of release to effect a therapeutic outcome. In one embodiment, the polynucleotides can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the polynucleotides described herein can be encapsulated in a therapeutic nanoparticle, referred to herein as “therapeutic nanoparticle polynucleotides.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle polynucleotide can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle polynucleotide can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the polynucleotides described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the polynucleotides can be formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.
In some embodiments, the polynucleotides described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, or less than 975 um.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

d. Lipidoids

In some embodiments, the compositions or formulations of the present disclosure comprise a delivery agent, e.g., a lipidoid. The polynucleotides described herein (e.g., a polynucleotide comprising a nucleotide sequence encoding a first polypeptide) can be formulated with lipidoids. Complexes, micelles, liposomes or particles can be prepared containing these lipidoids and therefore to achieve an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C12-200 (including derivatives and variants), and MD1, can be tested for in vivo activity. The lipidoid “98N12-5” is disclosed by Akinc et al., Mol Ther. 2009 17:872-879. The lipidoid “C12-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the polynucleotides described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to polynucleotides. Lipidoids and polynucleotide formulations comprising lipidoids are described in Intl. Pub. No. WO 2015051214 (herein incorporated by reference in its entirety.

e. Hyaluronidase

In some embodiments, the polynucleotides described herein (e.g., a polynucleotide comprising a nucleotide sequence encoding a first polypeptide) and hyaluronidase for injection (e.g., intramuscular or subcutaneous injection). Hyaluronidase catalyzes the hydrolysis of hyaluronan, which is a constituent of the interstitial barrier. Hyaluronidase lowers the viscosity of hyaluronan, thereby increases tissue permeability (Frost, Expert Opin. Drug Deliv. (2007) 4:427-440). Alternatively, the hyaluronidase can be used to increase the number of cells exposed to the polynucleotides administered intramuscularly, or subcutaneously.

f. Nanoparticle Mimics

In some embodiments, the polynucleotides described herein (e.g., a polynucleotide comprising a nucleotide sequence encoding a first polypeptide) is encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example, the polynucleotides described herein can be encapsulated in a non-viron particle that can mimic the delivery function of a virus (see e.g., Intl. Pub. No. WO2012006376 and U.S. Pub. Nos. US20130171241 and US20130195968, each of which is herein incorporated by reference in its entirety).

g. Self-Assembled Nanoparticles, or Self-Assembled Macromolecules

In some embodiments, the compositions or formulations of the present disclosure comprise the polynucleotides described herein (e.g., a polynucleotide comprising a nucleotide sequence encoding a first polypeptide) in self-assembled nanoparticles, or amphiphilic macromolecules (AMs) for delivery. AMs comprise biocompatible amphiphilic polymers that have an alkylated sugar backbone covalently linked to poly(ethylene glycol). In aqueous solution, the AMs self-assemble to form micelles. Nucleic acid self-assembled nanoparticles are described in Intl. Appl. No. PCT/US2014/027077, and AMs and methods of forming AMs are described in U.S. Pub. No. US20130217753, each of which is herein incorporated by reference in its entirety.

h. Cations and Anions
In some embodiments, the compositions or formulations of the present disclosure comprise the polynucleotides described herein (e.g., a polynucleotide comprising a nucleotide sequence encoding a first polypeptide) and a cation or anion, such as Zn2+, Ca2+, Cu2+, Mg2+ and combinations thereof. Exemplary formulations can include polymers and a polynucleotide complexed with a metal cation as described in, e.g., U.S. Pat. Nos. 6,265,389 and 6,555,525, each of which is herein incorporated by reference in its entirety. In some embodiments, cationic nanoparticles can contain a combination of divalent and monovalent cations. The delivery of polynucleotides in cationic nanoparticles or in one or more depot comprising cationic nanoparticles can improve polynucleotide bioavailability by acting as a long-acting depot and/or reducing the rate of degradation by nucleases.
i. Amino Acid Lipids

In some embodiments, the compositions or formulations of the present disclosure comprise the polynucleotides described herein (e.g., a polynucleotide comprising a nucleotide sequence encoding a first polypeptide) that is formulation with an amino acid lipid. Amino acid lipids are lipophilic compounds comprising an amino acid residue and one or more lipophilic tails. Non-limiting examples of amino acid lipids and methods of making amino acid lipids are described in U.S. Pat. No. 8,501,824. The amino acid lipid formulations can deliver a polynucleotide in releasable form that comprises an amino acid lipid that binds and releases the polynucleotides. As a non-limiting example, the release of the polynucleotides described herein can be provided by an acid-labile linker as described in, e.g., U.S. Pat. Nos. 7,098,032, 6,897,196, 6,426,086, 7,138,382, 5,563,250, and 5,505,931, each of which is herein incorporated by reference in its entirety.

### I. Interpolyelectrolyte Complexes

In some embodiments, the compositions or formulations of the present disclosure comprise the polynucleotides described herein (e.g., a polynucleotide comprising a nucleotide sequence encoding a first polypeptide) in an interpolyelectrolyte complex. Interpolyelectrolyte complexes are formed when charge-dynamic polymers are complexed with one or more anionic molecules. Non-limiting examples of charge-dynamic polymers and interpolyelectrolyte complexes and methods of making interpolyelectrolyte complexes are described in U.S. Pat. No. 8,524,368, herein incorporated by reference in its entirety.
k. Crystalline Polymeric Systems

In some embodiments, the compositions or formulations of the present disclosure comprise the polynucleotides described herein (e.g., a polynucleotide comprising a nucleotide sequence encoding a first polypeptide) in crystalline polymeric systems. Crystalline polymeric systems are polymers with crystalline moieties and/or terminal units comprising crystalline moieties. Exemplary polymers are described in U.S. Pat. No. 8,524,259 (herein incorporated by reference in its entirety).

l. Polymers, Biodegradable Nanoparticles, and Core-Shell Nanoparticles

In some embodiments, the compositions or formulations of the present disclosure comprise the polynucleotides described herein (e.g., a polynucleotide comprising a nucleotide sequence encoding a first polypeptide) and a natural and/or synthetic polymer. The polymers include, but not limited to, polyethenes, polyethylene glycol (PEG), poly(1-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, elastic biodegradable polymer, biodegradable copolymer, biodegradable polyester copolymer, biodegradable polyester copolymer, multiblock copolymers, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyureas, polystyrenes, polyamines, polylysine, poly(ethylene imine), poly(serine ester), poly(L-lactide-co-L-lysine), poly(4-hydroxy-L-proline ester), amine-containing polymers, dextran polymers, dextran polymer derivatives or combinations thereof.

Exemplary polymers include, DYNAMIC POLYCONJUGATE® (Arrowhead Research Corp., Pasadena, CA) formulations from MIRUS® Bio (Madison, WI) and Roche Madison (Madison, WI), PHASERX™ polymer formulations such as, without limitation, SMARTT POLYMER TECHNOLOGY™ (PHASERX®, Seattle, WA), DMRI/DOPE, poloxamer, VAXFECTIN® adjuvant from Vical (San Diego, CA), chitosan, cyclodextrin from Calando Pharmaceuticals (Pasadena, CA), dendrimers and poly(lactic-co-glycolic acid) (PLGA) polymers. RONDEL™ (RNAi/Oligonucleotide Nanoparticle Delivery) polymers (Arrowhead Research Corporation, Pasadena, CA) and pH responsive co-block polymers such as PHASERX® (Seattle, WA).

The polymer formulations allow a sustained or delayed release of the polynucleotide (e.g., following intramuscular or subcutaneous injection). The altered release profile for the polynucleotide can result in, for example, translation of an encoded protein over an extended period of time. The polymer formulation can also be used to increase the stability of the polynucleotide. Sustained release formulations can include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc. Deerfield, IL), PEG-based sealants, and COSEAL® (Baxter International, Inc. Deerfield, IL).

As a non-limiting example modified mRNA can be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers that are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C.

As a non-limiting example, the polynucleotides described herein can be formulated with the polymeric compound of PEG grafted with PLL as described in U.S. Pat. No. 6,177,274. As another non-limiting example, the polynucleotides described herein can be formulated with a block copolymer such as a PLGA-PEG block copolymer (see e.g., U.S. Pub. No. US20120004293 and U.S. Pat. Nos. 8,236,330 and 8,246,968), or a PLGA-PEG-PLGA block copolymer (see e.g., U.S. Pat. No. 6,004,573). Each of the references is herein incorporated by reference in its entirety.

In some embodiments, the polynucleotides described herein can be formulated with at least one amine-containing polymer such as, but not limited to polylysine, polyethylene imine, poly(amidoamine) dendrimers, poly(amine-co-esters) or combinations thereof. Exemplary polyamine polymers and their use as delivery agents are described in, e.g., U.S. Pat. Nos. 8,460,696, 8,236,280, each of which is herein incorporated by reference in its entirety.

In some embodiments, the polynucleotides described herein can be formulated in a biodegradable cationic lipopolymer, a biodegradable polymer, or a biodegradable copolymer, a biodegradable polyester copolymer, a biodegradable polyester polymer, a linear biodegradable copolymer, PAGA, a biodegradable cross-linked cationic multi-block copolymer or combinations thereof as described in, e.g., U.S. Pat. Nos. 6,696,038, 6,517,869, 6,267,987, 6,217,912, 6,652,886, 8,057,821, and 8,444,992; U.S. Pub. Nos. US20030073619, US20040142474, US20100004315, US2012009145 and US20130195920; and Intl Pub. Nos. WO2006063249 and WO2013086322, each of which is herein incorporated by reference in its entirety.

In some embodiments, the polynucleotides described herein can be formulated in or with at least one cyclodextrin polymer as described in U.S. Pub. No. US20130184453. In some embodiments, the polynucleotides described herein can be formulated in or with at least one crosslinked cation-binding polymers as described in Intl. Pub. Nos. WO2013106072, WO2013106073 and WO2013106086. In some embodiments, the polynucleotides described herein can be formulated in or with at least PEGylated albumin polymer as described in U.S. Pub. No. US20130231287. Each of the references is herein incorporated by reference in its entirety.

In some embodiments, the polynucleotides disclosed herein can be formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle for delivery (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in their entireties). As a non-limiting example, the nanoparticle can comprise a plurality of polymers such as, but not limited to hydrophilic-hydrophobic polymers (e.g., PEG-PLGA), hydrophobic polymers (e.g., PEG) and/or hydrophilic polymers (Intl. Pub. No. WO20120225129, herein incorporated by reference in its entirety).

The use of core-shell nanoparticles has additionally focused on a high-throughput approach to synthesize cationic cross-linked nanogel cores and various shells (Siegwart et al., Proc Natl Acad Sci USA. 2011 108:12996-13001; herein incorporated by reference in its entirety). The complexation, delivery, and internalization of the polymeric nanoparticles can be precisely controlled by altering the chemical composition in both the core and shell components of the nanoparticle. For example, the core-shell nanoparticles can efficiently deliver siRNA to mouse hepatocytes after they covalently attach cholesterol to the nanoparticle.

In some embodiments, a hollow lipid core comprising a middle PLGA layer and an outer neutral lipid layer containing PEG can be used to delivery of the polynucleotides as described herein. In some embodiments, the lipid nanoparticles can comprise a core of the polynucleotides disclosed herein and a polymer shell, which is used to protect the polynucleotides in the core. The polymer shell can be any of the polymers described herein and are known in the art. The polymer shell can be used to protect the polynucleotides in the core.

Core-shell nanoparticles for use with the polynucleotides described herein are described in U.S. Pat. No. 8,313,777 or Intl. Pub. No. WO2013124867, each of which is herein incorporated by reference in their entirety.

m. Peptides and Proteins
In some embodiments, the compositions or formulations of the present disclosure comprise the polynucleotides described herein (e.g., a polynucleotide comprising a nucleotide sequence encoding a first polypeptide) that is formulated with peptides and/or proteins to increase transfection of cells by the polynucleotide, and/or to alter the biodistribution of the polynucleotide (e.g., by targeting specific tissues or cell types), and/or increase the translation of encoded protein (e.g., Intl. Pub. Nos. WO2012110636 and WO2013123298. In some embodiments, the peptides can be those described in U.S. Pub. Nos. US20130129726, US20130137644 and US20130164219. Each of the references is herein incorporated by reference in its entirety.
n. Conjugates
In some embodiments, the compositions or formulations of the present disclosure comprise the polynucleotides described herein (e.g., a polynucleotide comprising a nucleotide sequence encoding a first polypeptide) that is covalently linked to a carrier or targeting group, or including two encoding regions that together produce a fusion protein (e.g., bearing a targeting group and therapeutic protein or peptide) as a conjugate. The conjugate can be a peptide that selectively directs the nanoparticle to neurons in a tissue or organism, or assists in crossing the blood-brain barrier.

The conjugates include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid, an oligonucleotide (e.g., an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, the conjugate can function as a carrier for the polynucleotide disclosed herein. The conjugate can comprise a cationic polymer such as, but not limited to, polyamine, polylysine, polyalkylenimine, and polyethylenimine that can be grafted to with poly(ethylene glycol). Exemplary conjugates and their preparations are described in U.S. Pat. No. 6,586,524 and U.S. Pub. No. US20130211249, each of which herein is incorporated by reference in its entirety.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as an endothelial cell or bone cell. Targeting groups can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent fructose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein. As a non-limiting example, the targeting group can be a glutathione receptor (GR)-binding conjugate for targeted delivery across the blood-central nervous system barrier as described in, e.g., U.S. Pub. No. US2013021661012 (herein incorporated by reference in its entirety).

In some embodiments, the conjugate can be a synergistic biomolecule-polymer conjugate, which comprises a long-acting continuous-release system to provide a greater therapeutic efficacy. The synergistic biomolecule-polymer conjugate can be those described in U.S. Pub. No. US20130195799. In some embodiments, the conjugate can be an aptamer conjugate as described in Intl. Pat. Pub. No. WO2012040524. In some embodiments, the conjugate can be an amine containing polymer conjugate as described in U.S. Pat. No. 8,507,653. Each of the references is herein incorporated by reference in its entirety. In some embodiments, the polynucleotides can be conjugated to SMARTT POLYMER TECHNOLOGY® (PHASERX®, Inc. Seattle, WA).

In some embodiments, the polynucleotides described herein are covalently conjugated to a cell penetrating polypeptide, which can also include a signal sequence or a targeting sequence. The conjugates can be designed to have increased stability, and/or increased cell transfection; and/or altered the biodistribution (e.g., targeted to specific tissues or cell types).

In some embodiments, the polynucleotides described herein can be conjugated to an agent to enhance delivery. In some embodiments, the agent can be a monomer or polymer such as a targeting monomer or a polymer having targeting blocks as described in Intl. Pub. No. WO2011062965. In some embodiments, the agent can be a transport agent covalently coupled to a polynucleotide as described in, e.g., U.S. Pat. Nos. 6,835,393 and 7,374,778. In some embodiments, the agent can be a membrane barrier transport enhancing agent such as those described in U.S. Pat. Nos. 7,737,108 and 8,003,129. Each of the references is herein incorporated by reference in its entirety.

### Pharmaceutically Acceptable Excipients

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, MD, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof. A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Production of Lipid Nanoparticle Compositions

### A. Production of Nanoparticle Compositions

In order to investigate safe and efficacious nanoparticle compositions for use in the delivery of polynucleotides of the disclosure to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the polynucleotides of the disclosure and the other has the lipid components.

Lipid compositions are prepared by combining a lipid according to Formulae (I), (IA), (IB), (II), (IIa), (IIb), (IIc), (IId), (IIe), (IIf), (IIg), (III), (IIIa1), (IIIa2), (IIIIa), (IIIa4), (IIIa5), (IIIa6), (IIIa7), or (IIIa8) or a non-cationic helper lipid (such as DOPE, or DSPC obtainable from Avanti Polar Lipids, Alabaster, AL), a PEG lipid (such as 1,2 dimyristoyl sn glycerol methoxypolyethylene glycol, also known as PEG-DMG, obtainable from Avanti Polar Lipids, Alabaster, AL), and a phytosterol (optionally including a structural lipid such as cholesterol) at concentrations of about, e.g., 50 mM in a solvent, e.g., ethanol. Solutions should be refrigerated for storage at, for example, −20° C. Lipids are combined to yield desired molar ratios (see, for example, Table 5 below) and diluted with water and ethanol to a final lipid concentration of e.g., between about 5.5 mM and about 25 mM. Phytosterol* in Table 5 refers to phytosterol or optionally a combination of phytosterol and structural lipid such as beta-phytosterol and cholesterol.

TABLE 5
Exemplary formulations of Lipid Nanoparticle compositions

| Composition | |
|---|---|
| (mol %) | Components |
| 40:20:38.5:1.5 | Ionizable lipid:Phospholipid:Phytosterol*:PEG-DMG |
| 45:15:38.5:1.5 | Ionizable lipid:Phospholipid:Phytosterol*:PEG-DMG |
| 50:10:38.5:1.5 | Ionizable lipid:Phospholipid:Phytosterol*:PEG-DMG |
| 55:5:38.5:1.5 | Ionizable lipid:Phospholipid:Phytosterol*:PEG-DMG |
| 60:5:33.5:1.5 | Ionizable lipid:Phospholipid:Phytosterol*:PEG-DMG |
| 45:20:33.5:1.5 | Ionizable lipid:Phospholipid:Phytosterol*:PEG-DMG |
| 50:20:28.5:1.5 | Ionizable lipid:Phospholipid:Phytosterol*:PEG-DMG |
| 55:20:23.5:1.5 | Ionizable lipid:Phospholipid:Phytosterol*:PEG-DMG |
| 60:20:18.5:1.5 | Ionizable lipidd:Phospholipid:Phytosterol*:PEG-DMG |
| 40:15:43.5:1.5 | Ionizable lipid:Phospholipid:Phytosterol*:PEG-DMG |
| 50:15:33.5:1.5 | Ionizable lipid:Phospholipid:Phytosterol*:PEG-DMG |
| 55:15:28.5:1.5 | Ionizable lipid:Phospholipid:Phytosterol*:PEG-DMG |
| 60:15:23.5:1.5 | Ionizable lipid:Phospholipid:Phytosterol*:PEG-DMG |
| 40:10:48.5:1.5 | Ionizable lipid:Phospholipid:Phytosterol*:PEG-DMG |
| 45:10:43.5:1.5 | Ionizable lipid:Phospholipid:Phytosterol*:PEG-DMG |
| 55:10:33.5:1.5 | Ionizable lipid:Phospholipid:Phytosterol*:PEG-DMG |
| 60:10:28.5:1.5 | Ionizable lipidd:Phospholipid:Phytosterol*:PEG-DMG |
| 40:5:53.5:1.5 | Ionizable lipid:Phospholipid:Phytosterol*:PEG-DMG |
| 45:5:48.5:1.5 | Ionizable lipid:Phospholipid:Phytosterol*:PEG-DMG |
| 50:5:43.5:1.5 | Ionizable lipid:Phospholipid:Phytosterol*:PEG-DMG |
| 40:20:40:0 | Ionizable lipid:Phospholipid:Phytosterol*:PEG-DMG |
| 45:20:35:0 | Ionizable lipid:Phospholipid:Phytosterol*:PEG-DMG |
| 50:20:30:0 | Ionizable lipidd:Phospholipid:Phytosterol*:PEG-DMG |
| 55:20:25:0 | Ionizable lipid:Phospholipid:Phytosterol*:PEG-DMG |
| 60:20:20:0 | Ionizable lipid:Phospholipid:Phytosterol*:PEG-DMG |
| 40:15:45:0 | Ionizable lipid:Phospholipid:Phytosterol*:PEG-DMG |
| 45:15:40:0 | Ionizable lipid:Phospholipid:Phytosterol*:PEG-DMG |
| 50:15:35:0 | Ionizable lipid:Phospholipid:Phytosterol*:PEG-DMG |
| 55:15:30:0 | Ionizable lipidPhospholipid:Phytosterol*:PEG-DMG |
| 60:15:25:0 | Ionizable lipid:Phospholipid:Phytosterol*:PEG-DMG |
| 40:10:50:0 | Ionizable lipid:Phospholipid:Phytosterol*:PEG-DMG |
| 45:10:45:0 | Ionizable lipidPhospholipid:Phytosterol*:PEG-DMG |
| 50:0:48.5:1.5 | Ionizable lipid:Phospholipid:Phytosterol*:PEG-DMG |
| 50:10:40:0 | Ionizable lipid:Phospholipid:Phytosterol*:PEG-DMG |
| 55:10:35:0 | Ionizable lipid:Phospholipid:Phytosterol*:PEG-DMG |
| 60:10:30:0 | Ionizable lipidPhospholipid:Phytosterol*:PEG-DMG |

Nanoparticle compositions including polynucleotide(s) of the disclosure and a lipid component are prepared by combining the lipid solution with a solution including the polynucleotides of the disclosure at lipid component to polynucleotides wt:wt ratios between about 5:1 and about 50:1. The lipid solution is rapidly injected using a NanoAssemblr microfluidic based system at flow rates between about 10 ml/min and about 18 ml/min into the polynucleotides solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa. The first dialysis is carried out at room temperature for 3 hours. The formulations are then dialyzed overnight at 4° C. The resulting nanoparticle suspension is filtered through 0.2 μm sterile filters (Sarstedt, Nümbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a polynucleotide (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of the polynucleotides of the disclosure in the nanoparticle composition can be calculated based on the extinction coefficient of the polynucleotides used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, CA) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilabel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver polynucleotides of the disclosure to targeted cells, different nanoparticle compositions including a particular polynucleotide of the disclosure (for example, an mRNA) are prepared and administered to rodent populations. Mice are intravenously, intramuscularly, subcutaneously, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a polynucleotide of the disclosure in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of polynucleotides. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the polynucleotides by a given nanoparticle composition relative to other nanoparticle compositions or the absence thereof.

### Example 2: Delayed Onset of Expression of Reporter mRNA Expression In Vitro

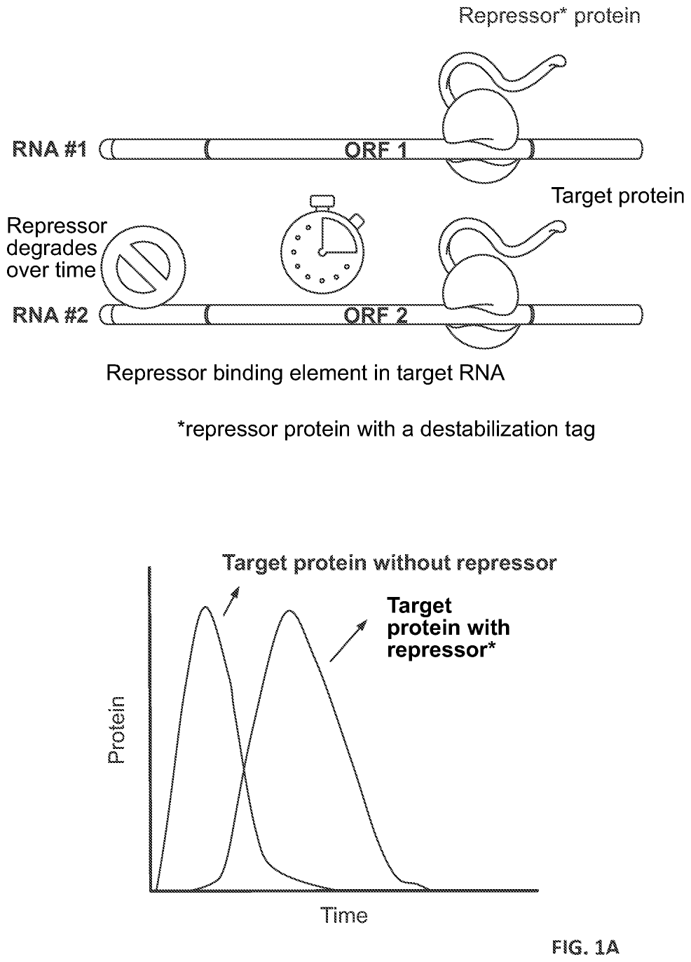

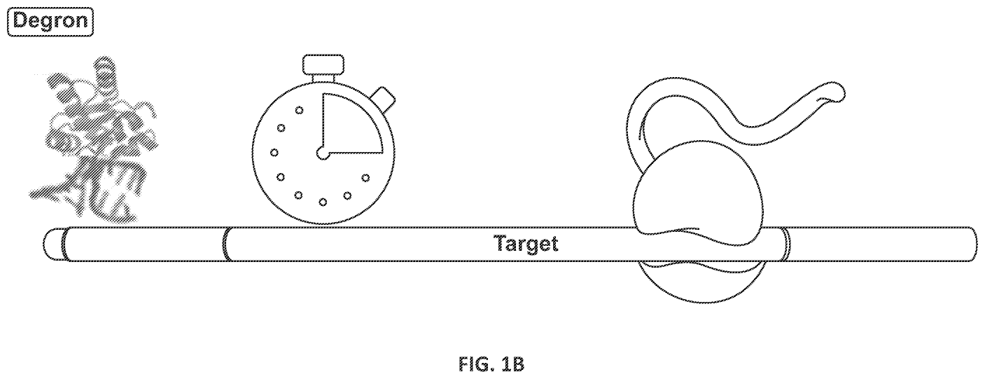

This example describes delayed expression of a target mRNA using the tethered timer-based repression of translation system described in FIGS. 1A-1B. L7Ae recruitment to target RNA has been shown to repress protein expression. The experiments described here demonstrate a proof of concept of a “Timer” system, which exploits the L7Ae repressor protein to achieve delayed onset of expression. Destabilization domains, as described in Chassin et al., Nat Commun 10:2013 (2019), were used to destabilize the L7Ae protein and hence limit the duration of its repression of the target RNA. After set durations as L7Ae protein is presumably degraded, the target RNA is free to start translation.

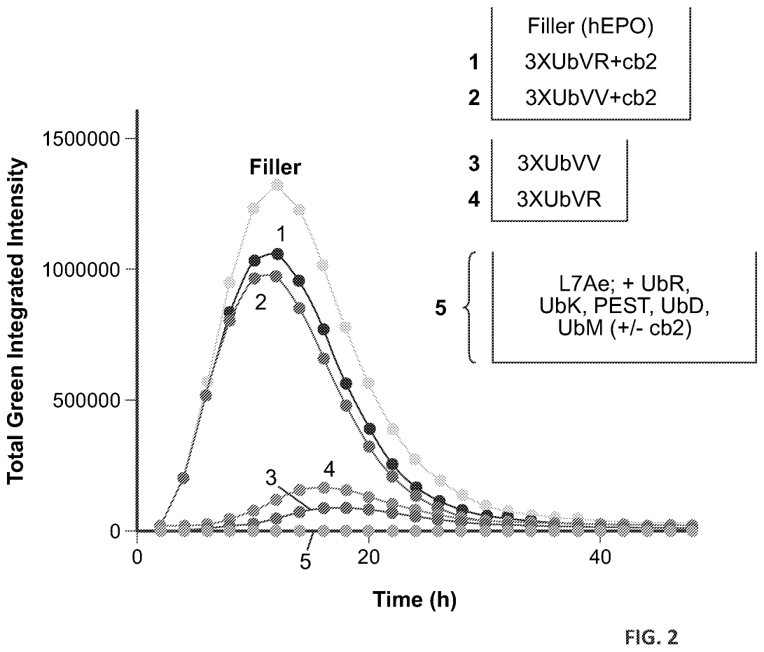

Destabilization domains as described in Chassin et al., Nat Commun 10:2013 (2019) were appended to L7Ae to hasten degradation of the Repressor. The Repressor as is or fused to degradation domains as noted were cotransfected into cells with target RNA containing the Repressor binding element. 3XUbVR and 3XUbVV tagged L7Ae showed delayed onset of deg-eGFP expression (FIG. 2). Other repressor variants with degrons showed complete or no repression for the duration of the experiment. Thus 3×UBVR-L7Ae and 3XUbVV fused L7Ae can act as timers for the target RNAs.

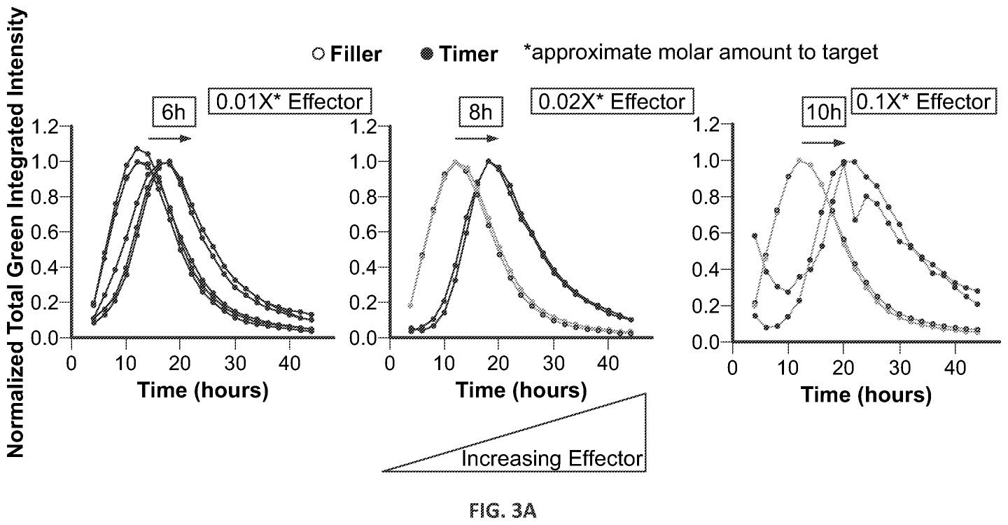

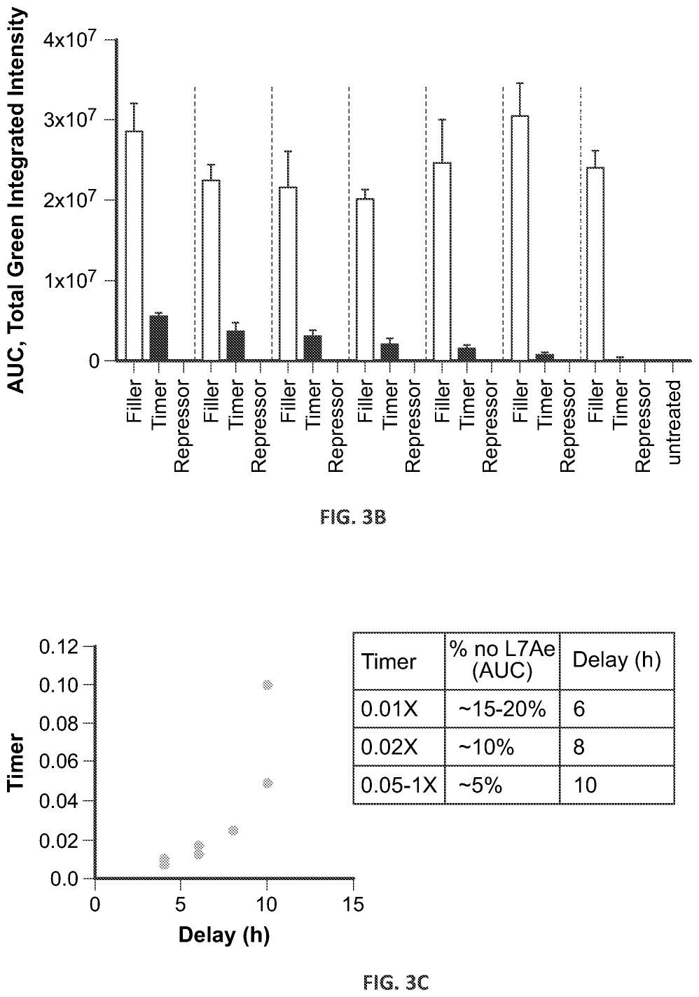

20 ng 5′kt deg-eGFP along with a range of effector concentrations were transfected in HeLa cells and imaged by Incucyte over 48 h. FIG. 3A shows increasing levels of Timer results in increased delay in HeLa cells. FIG. 3B shows deg-eGFP expression levels across different amounts of Timer. FIG. 3C shows delay interval correlates with fraction of Timer in the system. Further, the delay in expression of target RNA is greater with a higher concentration of effector in the system (See FIG. 3A).

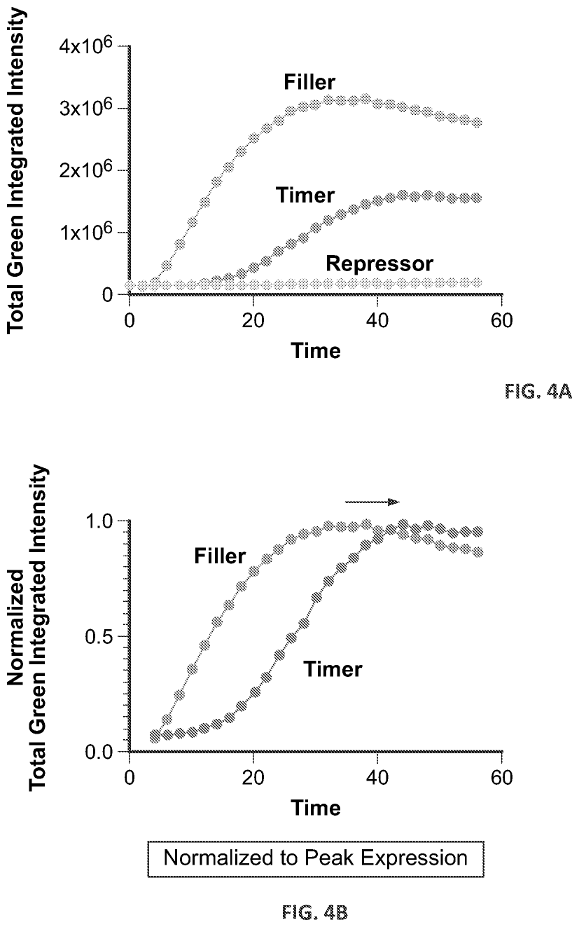

20 ng 5′kt deg-eGFP and effectors at 1:0.1 Target: Effector moles, were co-transfected in primary human hepatocytes and imaged by Incucyte over 60 h. FIGS. 4A-4B show total green integrated intensity (FIG. 4A) and normalized total green integrated intensity (FIG. 4B) over time from cells transfected with degGFP codelivered with Effectors and filler (EPO), timer (3XUbVR-L7Ae), or repressor (L7Ae) constructs.

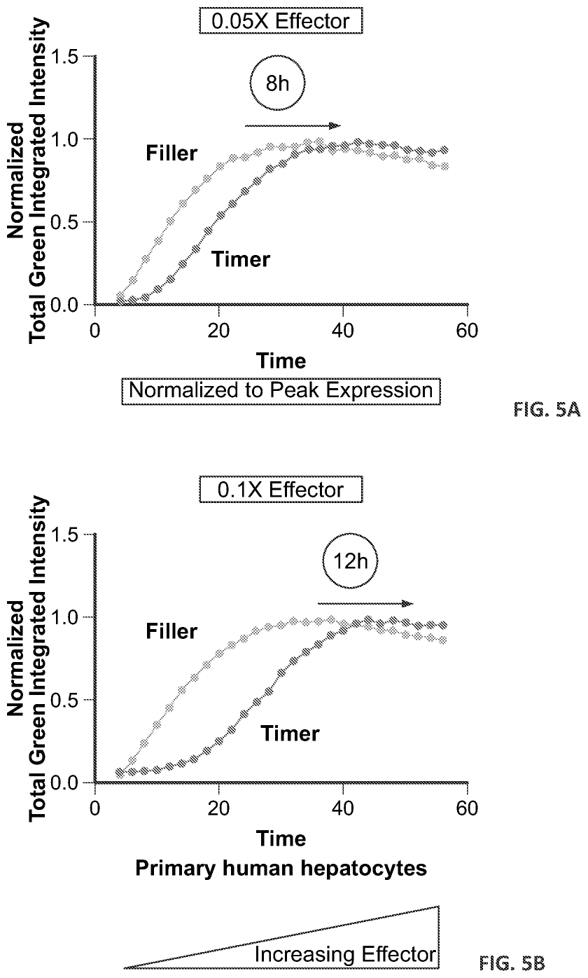

20 ng 5′kt deg-eGFP and effectors at 0.05× Target: Effector moles (FIG. 5A), 0.1× Target: Effector moles (FIG. 5B), and 0.2× Target: Effector moles (FIG. 5C) were co-transfected in primary human hepatocytes and imaged by Incucyte over 60 h. FIGS. 5A-5C show normalized total green integrated intensity over time from cells transfected with degGFP and effectors and filler or timer constructs.

FIGS. 3A-3C, FIGS. 4A-4B, and FIGS. 5A-5C show that destabilized L7Ae (3×UBvR fusion) can confer delayed onset of detectable expression from target RNA by using a tethered timer system.

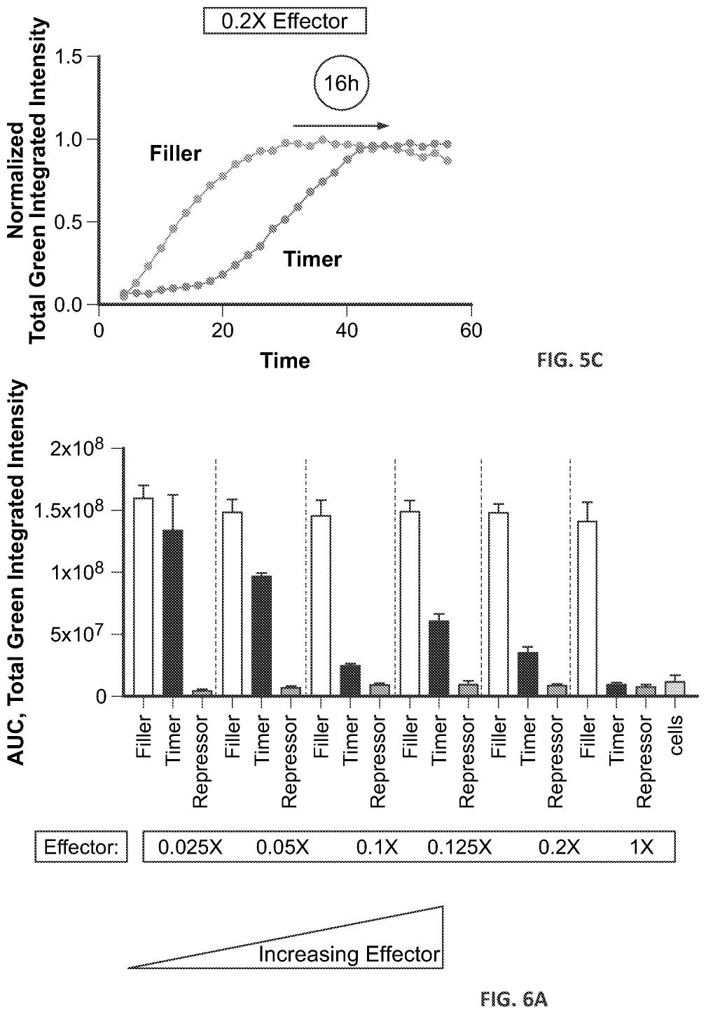

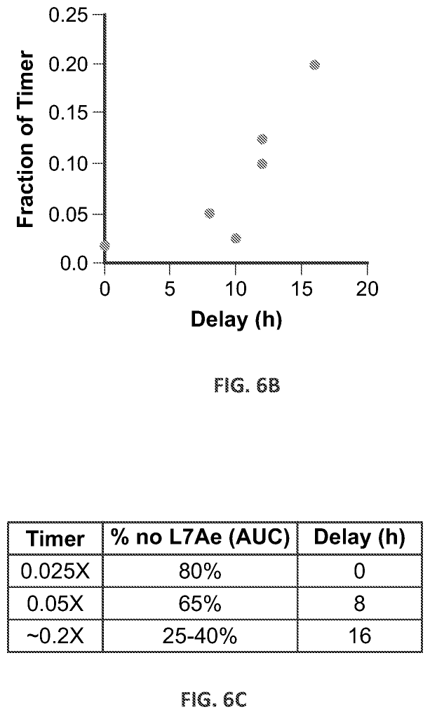

20 ng 5′kt deg-eGFP and effectors at various Target:Effector moles were co-transfected in primary human hepatocytes and imaged by Incucyte over 60 h. FIG. 6A shows the total green integrated intensity (area under curve, AUC) over time, at various Target:Effector ratios, from cells transfected with filler, timer or repressor. FIG. 6B shows fraction of timer vs delay in hours. FIG. 6C shows timer concentration vs. delay in hours, with increased amounts of L7Ae resulting in greater delay.

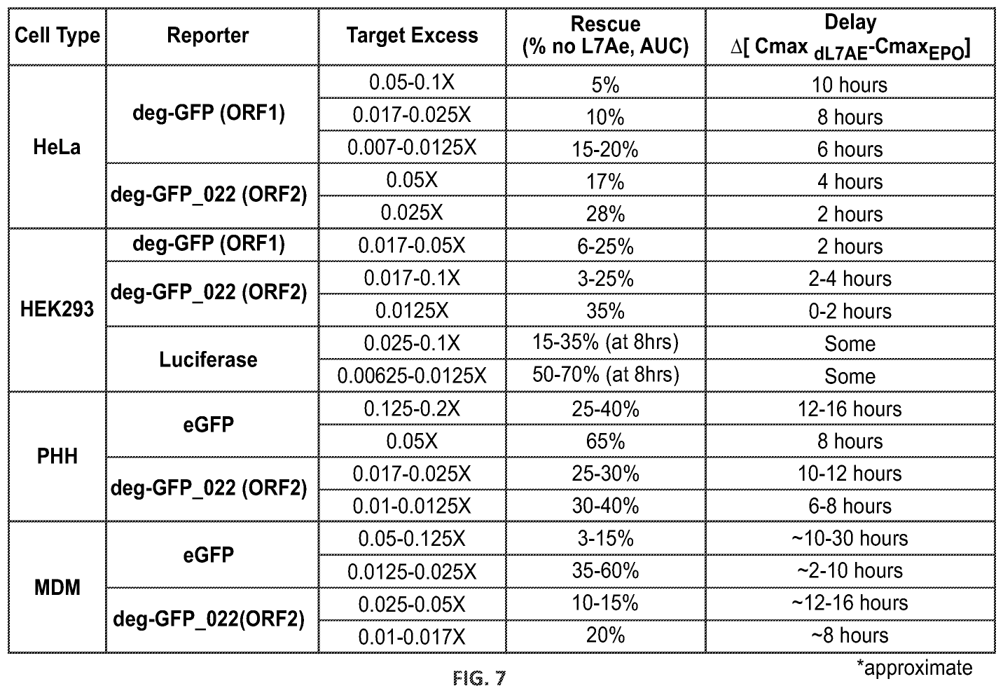

FIG. 7 is a table showing the levels of target protein rescue and delay seen with transfection of the various reporter system in the indicated cell types and at the indicated ratios (target excess).

### Example 3: Expression of Two Target RNAs Staggered By Using a Tethered Timer System

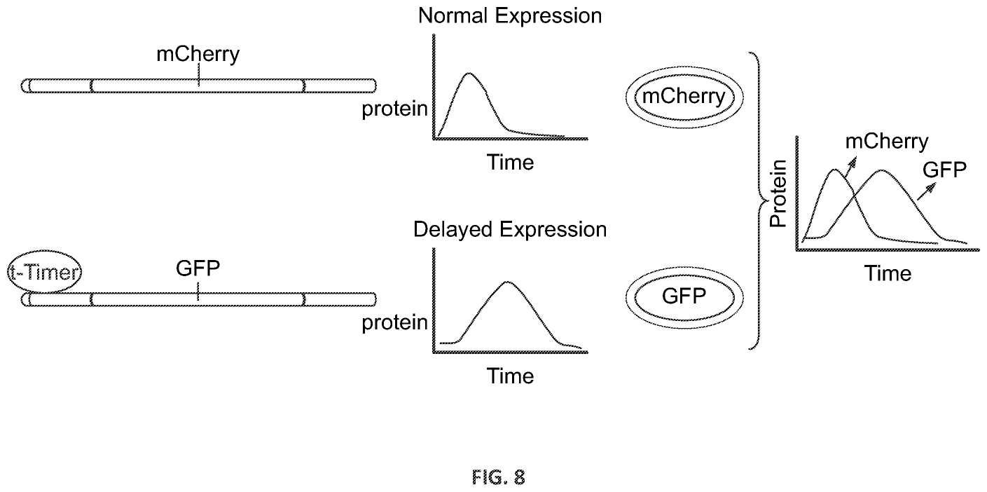

This example describes how to achieve delayed expression of one of two target mRNAs in cells using the tethered timer-based repression of translation system described in FIG. 8.

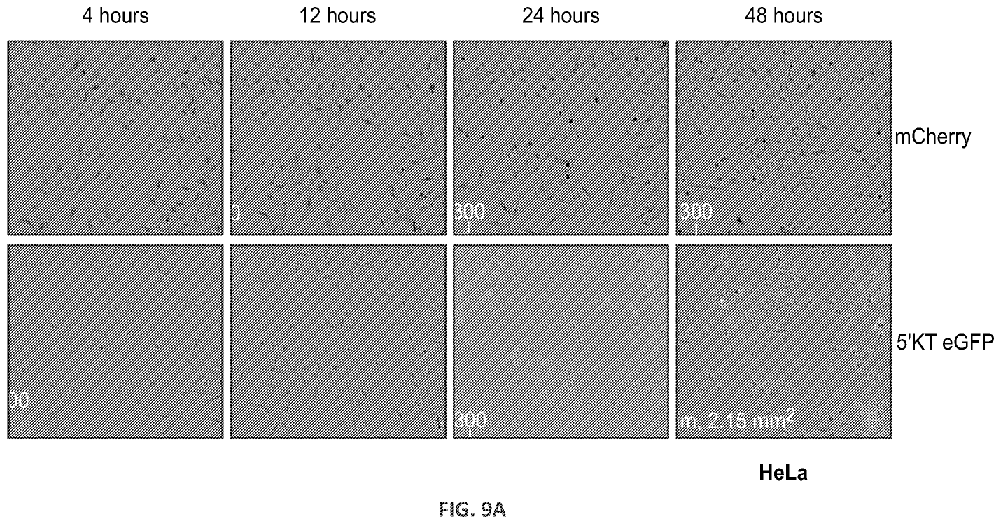

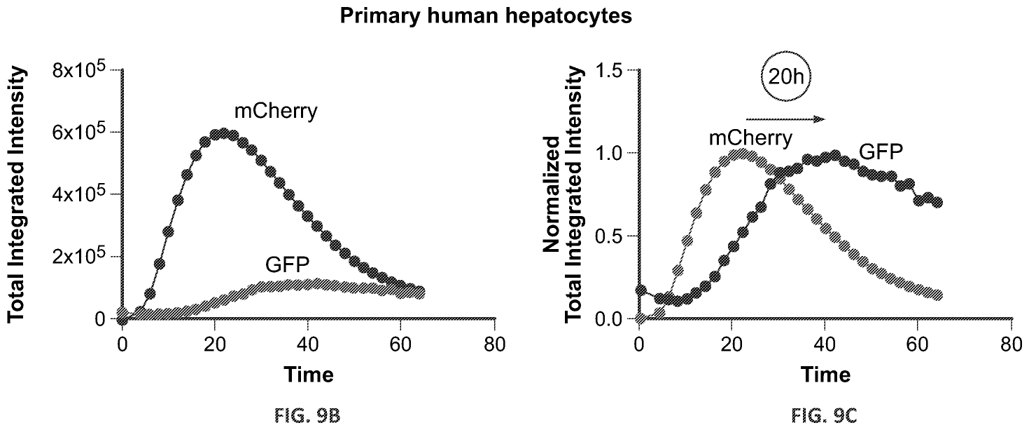

40 ng degmCherry and 10 ng 5′ KT eGFP were co-transfected in HeLa cells with Timer (3XUBVR-L7Ae) and imaged by Incucyte over 48 h. Expression of mCherry and GFP over time is shown in FIG. 9A.

40 ng mCherry, 10 ng 5′kt eGFP, and Timer (1:0.5 Target: Timer moles) were co-transfected in primary human hepatocytes and imaged by Incucyte red and green channels over 64 h. The expression of mCherry and GFP was staggered over time, with mCherry expression commencing at an earlier timepoint than eGFP expression (FIGS. 9B-9C).

### Example 4: mRNA Design Impacts Target Expression Levels

Sequence design of an RNA encoding the timer (e.g., L7Ae_3×UBvR) can be altered to result in different amounts of timer protein with the same amount of target RNAs. This in turn allows one to change the delay interval simply by changing the sequence design features of timer RNA.

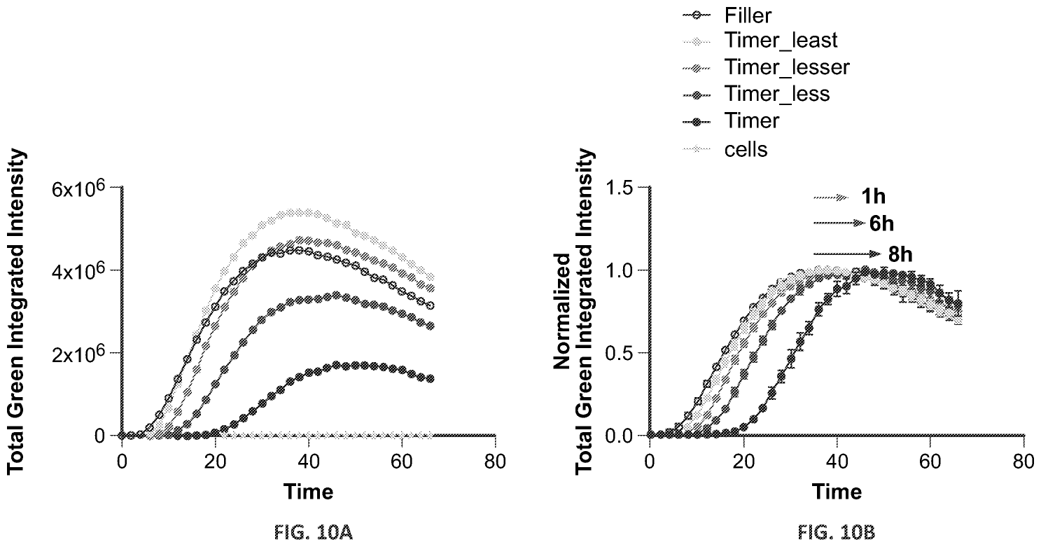

Timer sequence design variants and eGFP target mRNA were co-transfected (1:0.125 Target: Timer moles, 20 ng eGFP) in primary human hepatocytes and imaged by Incucyte over 66 h. The data in FIGS. 10A-10B indicate that the induced delay interval may be controlled by RNA design. L7Ae mRNA designs variants show the ability to modulate delay interval, at the same ratio of Timer, using sequence design variants.

### Example 5: Snu13 Repressor can be Used to Generate a Timer with No Apparent Toxicity

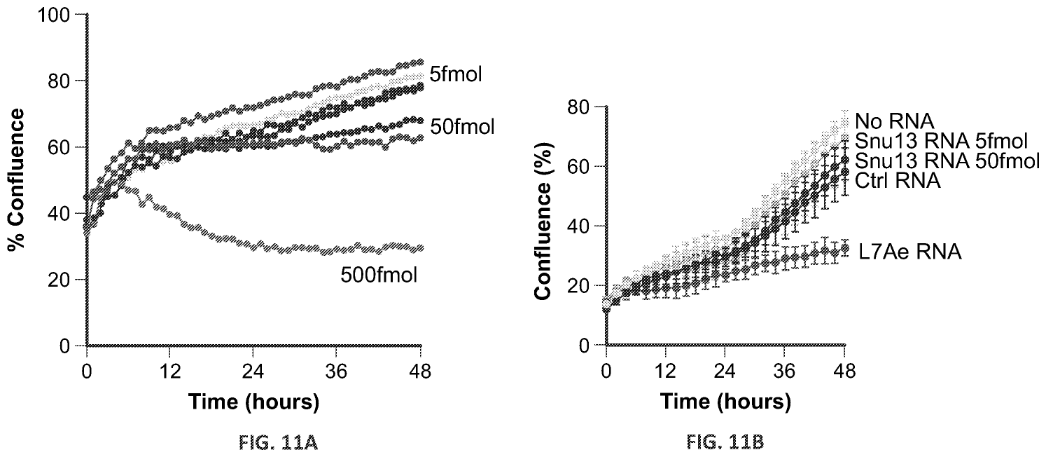

Snu13 RNAs at varying concentrations were tested in cells, along with L7Ae repressor. Snu13 was shown to be a viable alternative to L7Ae as shown in FIGS. 11A-11B.

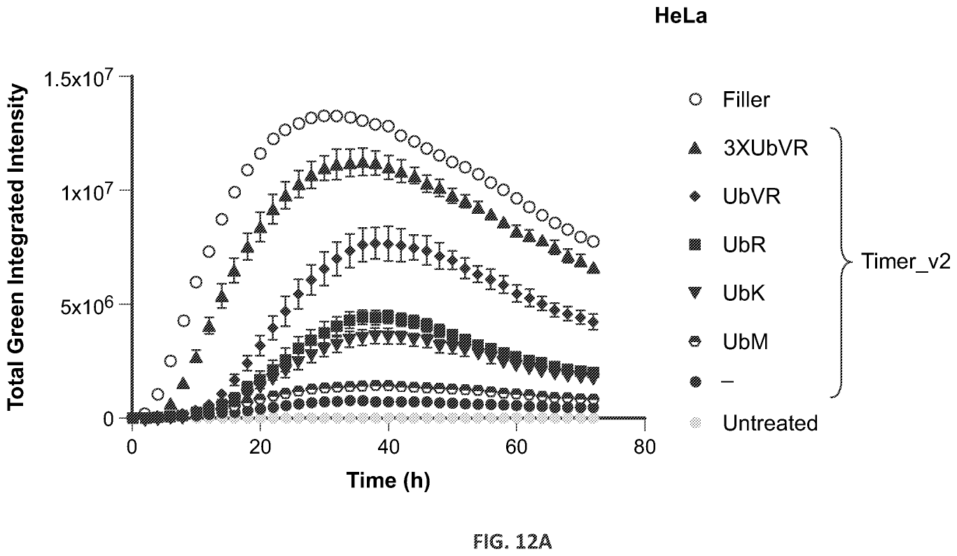

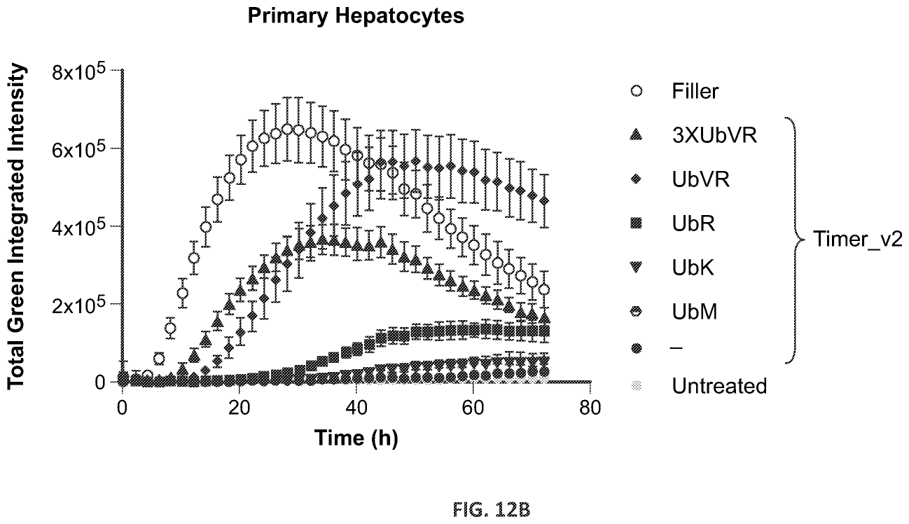

Destabilized Snu13 (with degron tag fusions) was assessed for its ability to delay onset of detectable expression from target RNA in HeLa cells and primary hepatocytes. Timer_v2 variants and a green fluorescent protein encoding target mRNA were co-transfected (10× Timer_v2, 10 ng KT green fluorescent protein) in HeLa cells and imaged by Incucyte over 75 h. Destabilized Snu13 (with degron tag fusions) delayed onset of detectable expression from target RNA in HeLa cells (FIG. 12A). Timer_v2 design variants and a green fluorescent protein target mRNA were co-transfected (5× Timer_v2, 10 ng KT green fluorescent protein) in primary human hepatocytes and imaged by Incucyte over 75 h. Destabilized Snu13 (with degron tag fusions) delayed onset of detectable expression from target RNA in primary hepatocytes (FIG. 12B).

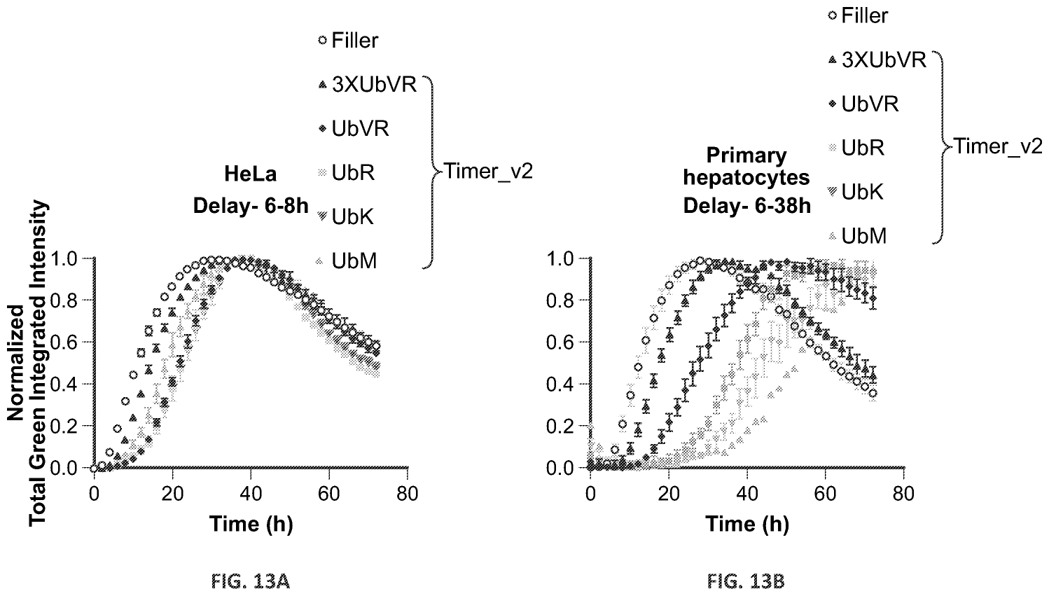

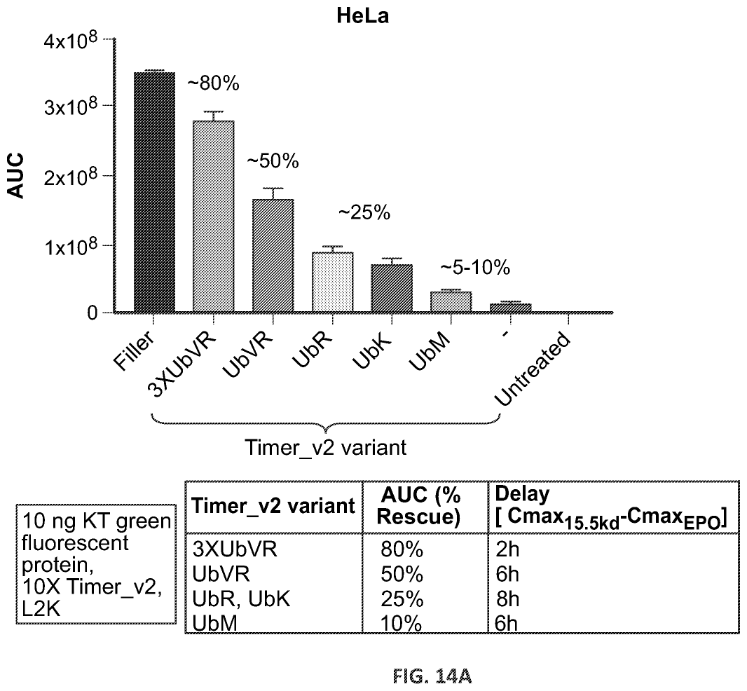

Snu13 (Timer_v2) design variants or a filler (EPO) and a green fluorescent protein target mRNA were co-transfected (10× Timer_v2, 10 ng KT green fluorescent protein) in HeLa cells and imaged by Incucyte over 75 h. FIG. 13A shows the normalized total green integrated intensity over time. Snu13 (Timer_v2) design variants and green fluorescent protein target mRNA were co-transfected (5× Timer_v2, 20 ng KT green fluorescent protein) in primary hepatocytes and imaged by Incucyte over 75 h. FIG. 13B shows the normalized total green integrated intensity over time.

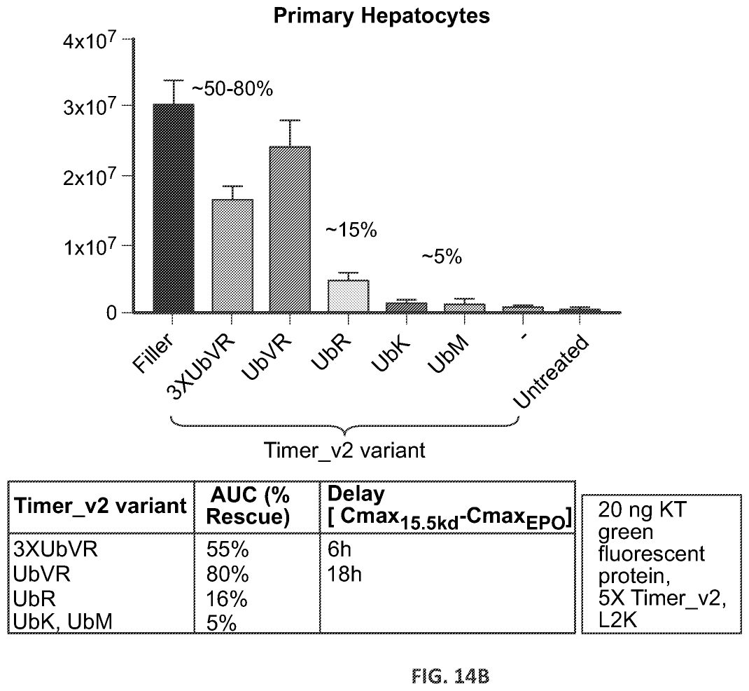

Snu13 (Timer_v2) design variants or a filler (EPO) and green fluorescent protein target mRNA were co-transfected (10× Timer_v2, 10 ng green fluorescent protein) in HeLa cells and imaged by Incucyte over 75 h. FIG. 14A shows the area under the curve indicating % rescue and the delay for various effectors in HeLa cells. Snu13 (Timer_v2) design variants or a filler (EPO) and green fluorescent protein target mRNA were co-transfected (5× Timer_v2, 20 ng KT green fluorescent protein) in primary hepatocytes and imaged by Incucyte over 75 h. FIG. 14B shows the area under the curve indicating % rescue and the delay for various effectors in primary hepatocytes.

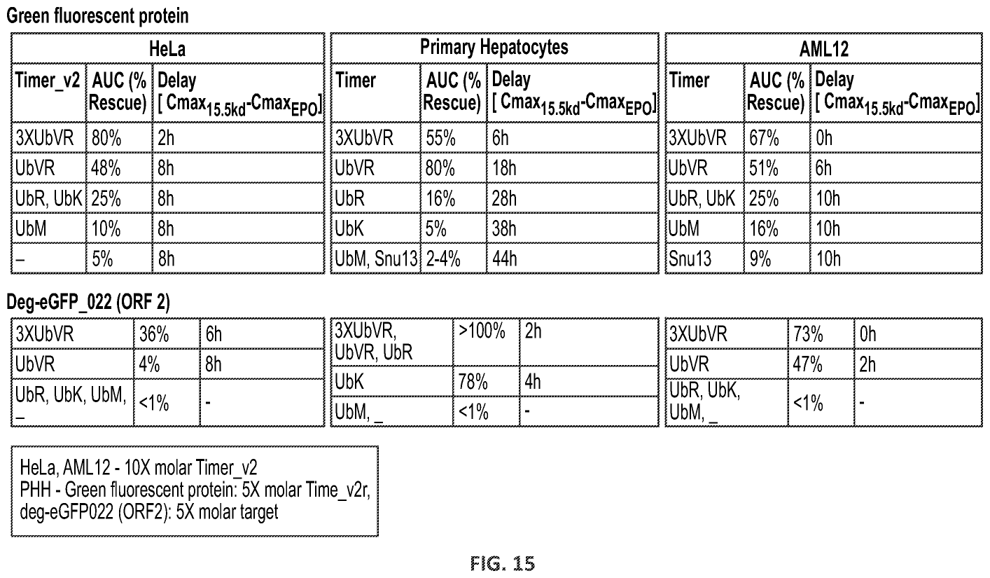

FIG. 15 summarizes reporter RNA translation delay and rescue observed in HeLa cells, primary hepatocytes, and AML12 cells over time.

TABLE 6
Miscellaneous sequences

| SEQ | | |
|---|---|---|
| ID | Sequence | |
| NO | information | Sequence |
| 151 | L7Ae (nt) | ATGTACGTGCGGTTCGAGGTGCCCGAGGACATGCAGAACGAGGCCCTGA |
| | | GCCTGCTGGAGAAGGTGCGGGAGAGCGGCAAGGTGAAGAAGGGCACCA |
| | | ACGAGACCACCAAGGCCGTGGAGCGGGGTCTGGCTAAGCTGGTGTACAT |
| | | CGCCGAGGACGTGGACCCTCCCGAGATCGTGGCCCACCTGCCCCTGCTG |
| | | TGCGAGGAGAAGAACGTGCCCTACATCTACGTGAAGAGTAAGAACGACC |
| | | TTGGCCGGGCCGTGGGCATCGAGGTCCCCTGCGCCAGCGCTGCCATCAT |
| | | CAACGAGGGCGAGCTGCGGAAGGAGCTGGGCAGCCTGGTGGAGAAGAT |
| | | CAAGGGCCTGCAGAAG |
| 152 | Timer_less (nt) | ATGTACGTCAGATTCGAGGTCCCAGAAGATATGCAGAACGAGGCATTAT |
| | | CTCTGCTCGAGAAGGTAAGAGAATCAGGGAAAGTCAAGAAGGGAACCA |
| | | ACGAAACAACTAAAGCAGTTGAACGCGGATTAGCCAAATTGGTGTACAT |
| | | TGCCGAAGACGTTGATCCACCTGAAATTGTGGCGCATTTACCTCTACTTT |
| | | GCGAGGAGAAGAACGTGCCGTATATTTACGTGAAGTCAAAGAACGATCT |
| | | GGGTAGAGCAGTTGGGATCGAAGTACCTTGTGCAAGTGCTGCCATTATC |
| | | AACGAAGGCGAACTCAGGAAAGAACTTGGCTCCTTAGTAGAGAAGATCA |
| | | AAGGGCTGCAGAAAGGTAAGCCTATCCCCAATCCACTACTTGGACTAGA |
| | | TTCTACC |
| 153 | Timer_lesser | ATGTACGTTAGATTTGAAGTACCAGAAGATATGCAGAACGAAGCTTTAT |
| | (nt) | CTTTATTGGAGAAAGTCAGAGAAAGTGGTAAAGTTAAGAAGGGAACAA |
| | Timer_least (nt) | ACGAGACAACTAAAGCTGTTGAGAGGGGATTAGCAAAGTTAGTTTATAT |
| | | TGCTGAAGACGTGGATCCTCCAGAAATTGTTGCACATTTGCCTTTACTTT |
| | | GTGAAGAGAAGAACGTTCCTTATATTTACGTGAAATCTAAGAACGATTT |
| | | AGGTAGAGCTGTTGGCATTGAAGTTCCCTGTGCATCCGCTGCTATAATTA |
| | | ACGAAGGAGAATTGAGAAAGGAACTTGGAAGTTTAGTTGAGAAGATTA |
| | | AAGGATTGCAGAAGGGTAAACCAATACCAAACCCTTTATTAGGTCTTGA |
| | | TTCTACC |
| 154 | PEST (nt) | ATGAGCCACGGCTTTCCTCCTGAGGTGGAGGAGCAGGACGACGGCACCC |
| | | TGCCCATGAGCTGCGCCCAGGAGAGCGGCATGGACCGGCATCCCGCCGC |
| | | CTGTGCCTCTGCCCGGATCAACGTG |
| 155 | UbR (nt) | ATGACCAGCCAGATTTTCGTCAAGACACTGACAGGCAAGACCATCACCC |
| | | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCCGAGCCAGC |
| 156 | UbD (nt) | ATGACCAGCCAGATTTTCGTCAAGACACTGACAGGCAAGACCATCACCC |
| | | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCGACGCCAGC |
| 157 | ubK (nt) | ATGACCAGCCAGATTTTCGTCAAGACACTGACAGGCAAGACCATCACCC |
| | | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCAAGGCCAGC |
| 158 | UbM (nt) | ATGACCAGCCAGATTTTCGTCAAGACACTGACAGGCAAGACCATCACCC |
| | | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCATGGCCAGC |
| 159 | cb2 (nt) | CTGAAGTACAAGCCCCTGTTGAAGATCAGCAAGAACTGCGAGGCCGCCA |
| | | TCCTGCGGGCCAGCAAGACCCGGCTGAACACCATCCGGGCCTACGGCAG |
| | | CACCGTGCCCAAGAGCAAGAGCTTCGAGCAGGACAGCCGGAAGCGGAC |
| | | CCAGAGCTGGACCGCCCTGCGGGTGGGTGCAATCCTGGCCGCCACCAGC |
| | | AGCGTGGCCTACCTGAACTGGCACAACGGCCAGATCGACAACGAGCCCA |
| | | AGCTGGACATGAACAAGCAGAAGATTAGCCCCGCCGAG |
| 160 | 3xUBVV (nt) | ATGACCAGCCAGATTTTCGTCAAGACACTGACAGGCAAGACCATCACCC |
| | | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGGTGGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTAGTGGCTAGCGCCAGTCA |
| | | GATCTTCGTGAAGACCCTCACCGGCAAGACGATTACCTTAGAGGTGGAG |
| | | CCTAGCGATACCATAGAGAACGTGAAGGCTAAGATCCAGGATAAGGAG |
| | | GGCATCCCTCCTGATCAACAACGGTTAATCTTTGCTGGCAAGCAGCTGGA |
| | | GGACGGACGGACCCTGAGCGATTACAATATCCAGAAGGAGAGCACCTTG |
| | | CACCTCGTGCTGCGGCTGAGAGGCGTCGTCGCCAGCGCCAGC |
| 161 | 3xUbVr (nt) | ATGACCAGCCAGATTTTCGTCAAGACACTGACAGGCAAGACCATCACCC |
| | | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGCGAGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTACGGGCTAGCGCCAGTC |
| | | AGATCTTCGTGAAGACCCTCACCGGCAAGACGATTACCTTAGAGGTGGA |
| | | GCCTAGCGATACCATAGAGAACGTGAAGGCTAAGATCCAGGATAAGGA |
| | | GGGCATCCCTCCTGATCAACAACGGTTAATCTTTGCTGGCAAGCAGCTGG |
| | | AGGACGGACGGACCCTGAGCGATTACAATATCCAGAAGGAGAGCACCTT |
| | | GCACCTCGTGCTGCGGCTGAGAGGCGTCCGTGCCAGCGCCAGC |
| 162 | deGGFP | ATGGTGTCCAAGGGTGAGGAATTGTTTACCGGGGTGGTGCCTATTCTCGT |
| | (ORF1) (nt) | CGAACTTGACGGGGATGTGAATGGACACAAGTTTTCGGTATCCGGAGAA |
| | | GGAGAGGGTGACGCCACATACGGAAAGCTTACACTCAAATTCATCTGTA |
| | | CGACGGGGAAACTGCCCGTACCCTGGCCTACGCTCGTAACCACGCTGAC |
| | | TTATGGAGTGCAGTGCTTTAGCAGATACCCCGACCATATGAAGCAGCAC |
| | | GACTTCTTCAAGTCGGCGATGCCCGAGGGGTACGTGCAAGAGAGGACCA |
| | | TTTTCTTCAAAGACGATGGCAATTACAAAACACGCGCAGAAGTCAAGTT |
| | | TGAGGGCGATACTCTGGTCAATCGGATCGAATTGAAGGGAATCGATTTC |
| | | AAAGAAGATGGAAACATCCTTGGCCATAAGCTCGAGTACAACTATAACT |
| | | CGCATAATGTCTATATCATGGCTGACAAGCAGAAAAACGGTATCAAAGT |
| | | CAACTTTAAGATCCGACACAATATTGAGGACGGTTCGGTGCAGCTTGCG |
| | | GACCACTATCAACAGAATACGCCGATTGGGGATGGTCCGGTCCTTTTGCC |
| | | GGATAACCATTATCTCTCAACCCAGTCAGCCCTGAGCAAAGATCCAAAC |
| | | GAGAAGAGGGACCACATGGTCTTGCTCGAATTCGTGACAGCGGCAGGGA |
| | | TCACTCTGGGAATGGACGAGTTGTACAAGAGATCTCGAGATATCAGCCA |
| | | TGGCTTCCCGCCGGCGGTGGCGGCGCAGGATGATGGCACGCTGCCCATG |
| | | TCTTGTGCCCAGGAGAGCGGGATGGACCGTCACCCTGCAGCCTGTGCTTC |
| | | TGCTAGGATCAATGTG |
| 163 | deGGFP22 | ATGGTGAGCAAGGGCGAGGAGCTGTTCACCGGCGTGGTGCCCATCCTGG |
| | (ORF2) (nt) | TGGAGCTGGACGGCGACGTGAACGGCCACAAGTTCAGCGTGTCCGGCGA |
| | | GGGCGAGGGCGACGCCACCTACGGCAAGCTGACCCTGAAGTTCATCTGC |
| | | ACCACCGGGAAGCTGCCCGTGCCCTGGCCCACCCTGGTGACCACCCTGA |
| | | CCTACGGGGTGCAGTGCTTCAGCAGGTACCCCGACCACATGAAGCAGCA |
| | | CGACTTCTTCAAGAGCGCCATGCCCGAGGGCTACGTGCAGGAGCGCACC |
| | | ATCTTCTTCAAGGACGACGGGAACTACAAGACCCGCGCCGAGGTGAAGT |
| | | TCGAGGGGGACACCCTGGTGAACCGGATCGAGCTGAAGGGGATCGACTT |
| | | CAAGGAGGACGGCAACATCCTGGGGCACAAGCTGGAGTACAACTACAA |
| | | CTCCCACAACGTGTACATCATGGCCGACAAGCAGAAGAACGGCATCAAG |
| | | GTGAACTTCAAGATCCGGCACAACATCGAGGACGGCAGCGTGCAGCTGG |
| | | CCGACCACTACCAGCAGAACACCCCCATCGGCGACGGCCCCGTGCTGCT |
| | | GCCCGACAACCACTACCTGAGCACCCAGAGCGCCCTGAGCAAGGACCCC |
| | | AACGAGAAGCGGGACCACATGGTGCTGCTGGAGTTCGTGACCGCCGCCG |
| | | GCATCACCCTGGGCATGGACGAGCTGTACAAGCGCTCCCGGGACATCTC |
| | | CCACGGCTTCCCACCCGCCGTGGCCGCTCAGGACGACGGCACCCTGCCC |
| | | ATGTCCTGCGCTCAGGAGAGCGGGATGGACCGCCACCCCGCCGCCTGCG |
| | | CCTCCGCCCGGATCAACGTG |
| 164 | mCherry (nt) | ATGGTATCCAAGGGGGAGGAGGACAACATGGCGATCATCAAGGAGTTC |
| | | ATGCGATTCAAGGTGCACATGGAAGGTTCGGTCAACGGACACGAATTTG |
| | | AAATCGAAGGAGAGGGTGAAGGAAGGCCCTATGAAGGGACACAGACCG |
| | | CGAAACTCAAGGTCACGAAAGGGGGACCACTTCCTTTCGCCTGGGACAT |
| | | TCTTTCGCCCCAGTTTATGTACGGGTCCAAAGCATATGTGAAGCATCCCG |
| | | CCGATATTCCTGACTATCTGAAACTCAGCTTTCCCGAGGGATTCAAGTGG |
| | | GAGCGGGTCATGAACTTTGAGGACGGGGGTGTAGTCACCGTAACCCAAG |
| | | ACTCAAGCCTCCAAGACGGCGAGTTCATCTACAAGGTCAAACTGCGGGG |
| | | GACTAACTTTCCGTCGGATGGGCCGGTGATGCAGAAGAAAACGATGGGA |
| | | TGGGAAGCGTCATCGGAGAGGATGTACCCAGAAGATGGTGCATTGAAGG |
| | | GGGAGATCAAGCAGAGACTGAAGTTGAAAGATGGGGGACATTATGATG |
| | | CCGAGGTGAAAACGACATACAAAGCGAAAAAGCCGGTGCAGCTTCCCG |
| | | GAGCGTATAATGTGAATATCAAGTTGGATATTACTTCACACAATGAGGA |
| | | CTACACAATTGTCGAACAGTACGAACGCGCTGAGGGTAGACACTCGACG |
| | | GGAGGCATGGACGAGTTGTACAAA |
| 165 | EPO (nt) | ATGGGAGTGCACGAGTGTCCCGCGTGGTTGTGGTTGCTGCTGTCGCTCTT |
| | | GAGCCTCCCACTGGGACTGCCTGTGCTGGGGGCACCACCCAGATTGATC |
| | | TGCGACTCACGGGTACTTGAGAGGTACCTTCTTGAAGCCAAAGAAGCCG |
| | | AAAACATCACAACCGGATGCGCCGAGCACTGCTCCCTCAATGAGAACAT |
| | | TACTGTACCGGATACAAAGGTCAATTTCTATGCATGGAAGAGAATGGAA |
| | | GTAGGACAGCAGGCCGTCGAAGTGTGGCAGGGGCTCGCGCTTTTGTCGG |
| | | AGGCGGTGTTGCGGGGTCAGGCCCTCCTCGTCAACTCATCACAGCCGTG |
| | | GGAGCCCCTCCAACTTCATGTCGATAAAGCGGTGTCGGGGCTCCGCAGC |
| | | TTGACGACGTTGCTTCGGGCTCTGGGCGCACAAAAGGAGGCTATTTCGC |
| | | CGCCTGACGCGGCCTCCGCGGCACCCCTCCGAACGATCACCGCGGACAC |
| | | GTTTAGGAAGCTTTTTAGAGTGTACAGCAATTTCCTCCGCGGAAAGCTGA |
| | | AATTGTATACTGGTGAAGCGTGTAGGACAGGGGATCGC |
| 166 | 3xUbVR_L7Ae_ | ATGACCAGCCAGATTTTCGTCAAGACACTGACAGGCAAGACCATCACCC |
| | cb2(2-95)_V5 | TGGAGGTGGAACCCTCCGACACCATTGAGAACGTCAAGGCCAAGATACA |
| | (nt) | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGCGAGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTACGGGCTAGCGCCAGTC |
| | | AGATCTTCGTGAAGACCCTCACCGGCAAGACGATTACCTTAGAGGTGGA |
| | | GCCTAGCGATACCATAGAGAACGTGAAGGCTAAGATCCAGGATAAGGA |
| | | GGGCATCCCTCCTGATCAACAACGGTTAATCTTTGCTGGCAAGCAGCTGG |
| | | AGGACGGACGGACCCTGAGCGATTACAATATCCAGAAGGAGAGCACCTT |
| | | GCACCTCGTGCTGCGGCTGAGAGGCGTCCGTGCCAGCGCCAGCTACGTG |
| | | CGGTTCGAGGTGCCCGAGGACATGCAGAACGAGGCCCTGAGCCTGCTGG |
| | | AGAAGGTGCGGGAGAGCGGCAAGGTGAAGAAGGGCACCAACGAGACCA |
| | | CCAAGGCCGTGGAGCGGGGTCTGGCTAAGCTGGTGTACATCGCCGAGGA |
| | | CGTGGACCCTCCCGAGATCGTGGCCCACCTGCCCCTGCTGTGCGAGGAG |
| | | AAGAACGTGCCCTACATCTACGTGAAGAGTAAGAACGACCTTGGCCGGG |
| | | CCGTGGGCATCGAGGTCCCCTGCGCCAGCGCTGCCATCATCAACGAGGG |
| | | CGAGCTGCGGAAGGAGCTGGGCAGCCTGGTGGAGAAGATCAAGGGCCT |
| | | GCAGAAGCTGAAGTACAAGCCCCTGTTGAAGATCAGCAAGAACTGCGAG |
| | | GCCGCCATCCTGCGGGCCAGCAAGACCCGGCTGAACACCATCCGGGCCT |
| | | ACGGCAGCACCGTGCCCAAGAGCAAGAGCTTCGAGCAGGACAGCCGGA |
| | | AGCGGACCCAGAGCTGGACCGCCCTGCGGGTGGGTGCAATCCTGGCCGC |
| | | CACCAGCAGCGTGGCCTACCTGAACTGGCACAACGGCCAGATCGACAAC |
| | | GAGCCCAAGCTGGACATGAACAAGCAGAAGATTAGCCCCGCCGAGATCC |
| | | CCAACCCACTGCTGGGCCTGGAC |
| 167 | 3xUbVV_L7Ae_ | ATGACCAGCCAGATTTTCGTCAAGACACTGACAGGCAAGACCATCACCC |
| | cb2(2-95)_V5 | TGGAGGTGGAACCCTCCGACACCATTGAGAACGTCAAGGCCAAGATACA |
| | (nt) | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGGTGGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTAGTGGCTAGCGCCAGTCA |
| | | GATCTTCGTGAAGACCCTCACCGGCAAGACGATTACCTTAGAGGTGGAG |
| | | CCTAGCGATACCATAGAGAACGTGAAGGCTAAGATCCAGGATAAGGAG |
| | | GGCATCCCTCCTGATCAACAACGGTTAATCTTTGCTGGCAAGCAGCTGGA |
| | | GGACGGACGGACCCTGAGCGATTACAATATCCAGAAGGAGAGCACCTTG |
| | | CACCTCGTGCTGCGGCTGAGAGGCGTCGTCGCCAGCGCCAGCTACGTGC |
| | | GGTTCGAGGTGCCCGAGGACATGCAGAACGAGGCCCTGAGCCTGCTGGA |
| | | GAAGGTGCGGGAGAGCGGCAAGGTGAAGAAGGGCACCAACGAGACCAC |
| | | CAAGGCCGTGGAGCGGGGTCTGGCTAAGCTGGTGTACATCGCCGAGGAC |
| | | GTGGACCCTCCCGAGATCGTGGCCCACCTGCCCCTGCTGTGCGAGGAGA |
| | | AGAACGTGCCCTACATCTACGTGAAGAGTAAGAACGACCTTGGCCGGGC |
| | | CGTGGGCATCGAGGTCCCCTGCGCCAGCGCTGCCATCATCAACGAGGGC |
| | | GAGCTGCGGAAGGAGCTGGGCAGCCTGGTGGAGAAGATCAAGGGCCTG |
| | | CAGAAGCTGAAGTACAAGCCCCTGTTGAAGATCAGCAAGAACTGCGAGG |
| | | CCGCCATCCTGCGGGCCAGCAAGACCCGGCTGAACACCATCCGGGCCTA |
| | | CGGCAGCACCGTGCCCAAGAGCAAGAGCTTCGAGCAGGACAGCCGGAA |
| | | GCGGACCCAGAGCTGGACCGCCCTGCGGGTGGGTGCAATCCTGGCCGCC |
| | | ACCAGCAGCGTGGCCTACCTGAACTGGCACAACGGCCAGATCGACAACG |
| | | AGCCCAAGCTGGACATGAACAAGCAGAAGATTAGCCCCGCCGAGATCCC |
| | | CAACCCACTGCTGGGCCTGGAC |
| 168 | UbR_L7Ae_cb2 | ATGACCAGCCAGATTTTCGTCAAGACACTGACAGGCAAGACCATCACCC |
| | (2-95)_V5 (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCCGAGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGCTGAAGTACAAGCCCCTGTTGAAGATCAGCAAGAACTG |
| | | CGAGGCCGCCATCCTGCGGGCCAGCAAGACCCGGCTGAACACCATCCGG |
| | | GCCTACGGCAGCACCGTGCCCAAGAGCAAGAGCTTCGAGCAGGACAGCC |
| | | GGAAGCGGACCCAGAGCTGGACCGCCCTGCGGGTGGGTGCAATCCTGGC |
| | | CGCCACCAGCAGCGTGGCCTACCTGAACTGGCACAACGGCCAGATCGAC |
| | | AACGAGCCCAAGCTGGACATGAACAAGCAGAAGATTAGCCCCGCCGAG |
| | | ATCCCCAACCCACTGCTGGGCCTGGAC |
| 169 | UbK_L7Ae_cb2 | ATGACCAGCCAGATTTTCGTCAAGACACTGACAGGCAAGACCATCACCC |
| | (2-95)_V5 (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCAAGGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGCTGAAGTACAAGCCCCTGTTGAAGATCAGCAAGAACTG |
| | | CGAGGCCGCCATCCTGCGGGCCAGCAAGACCCGGCTGAACACCATCCGG |
| | | GCCTACGGCAGCACCGTGCCCAAGAGCAAGAGCTTCGAGCAGGACAGCC |
| | | GGAAGCGGACCCAGAGCTGGACCGCCCTGCGGGTGGGTGCAATCCTGGC |
| | | CGCCACCAGCAGCGTGGCCTACCTGAACTGGCACAACGGCCAGATCGAC |
| | | AACGAGCCCAAGCTGGACATGAACAAGCAGAAGATTAGCCCCGCCGAG |
| | | ATCCCCAACCCACTGCTGGGCCTGGAC |
| 170 | PEST | ATGAGCCACGGCTTTCCTCCTGAGGTGGAGGAGCAGGACGACGGCACCC |
| | (mODC)_L7Ae_ | TGCCCATGAGCTGCGCCCAGGAGAGCGGCATGGACCGGCATCCCGCCGC |
| | cb2(2-95)_V5 | CTGTGCCTCTGCCCGGATCAACGTGTACGTGCGGTTCGAGGTGCCCGAG |
| | (nt) | GACATGCAGAACGAGGCCCTGAGCCTGCTGGAGAAGGTGCGGGAGAGC |
| | | GGCAAGGTGAAGAAGGGCACCAACGAGACCACCAAGGCCGTGGAGCGG |
| | | GGTCTGGCTAAGCTGGTGTACATCGCCGAGGACGTGGACCCTCCCGAGA |
| | | TCGTGGCCCACCTGCCCCTGCTGTGCGAGGAGAAGAACGTGCCCTACAT |
| | | CTACGTGAAGAGTAAGAACGACCTTGGCCGGGCCGTGGGCATCGAGGTC |
| | | CCCTGCGCCAGCGCTGCCATCATCAACGAGGGCGAGCTGCGGAAGGAGC |
| | | TGGGCAGCCTGGTGGAGAAGATCAAGGGCCTGCAGAAGCTGAAGTACA |
| | | AGCCCCTGTTGAAGATCAGCAAGAACTGCGAGGCCGCCATCCTGCGGGC |
| | | CAGCAAGACCCGGCTGAACACCATCCGGGCCTACGGCAGCACCGTGCCC |
| | | AAGAGCAAGAGCTTCGAGCAGGACAGCCGGAAGCGGACCCAGAGCTGG |
| | | ACCGCCCTGCGGGTGGGTGCAATCCTGGCCGCCACCAGCAGCGTGGCCT |
| | | ACCTGAACTGGCACAACGGCCAGATCGACAACGAGCCCAAGCTGGACAT |
| | | GAACAAGCAGAAGATTAGCCCCGCCGAGATCCCCAACCCACTGCTGGGC |
| | | CTGGAC |
| 171 | UbD_L7Ae_cb2 | ATGACCAGCCAGATTTTCGTCAAGACACTGACAGGCAAGACCATCACCC |
| | (2-95)_V5 (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCGACGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGCTGAAGTACAAGCCCCTGTTGAAGATCAGCAAGAACTG |
| | | CGAGGCCGCCATCCTGCGGGCCAGCAAGACCCGGCTGAACACCATCCGG |
| | | GCCTACGGCAGCACCGTGCCCAAGAGCAAGAGCTTCGAGCAGGACAGCC |
| | | GGAAGCGGACCCAGAGCTGGACCGCCCTGCGGGTGGGTGCAATCCTGGC |
| | | CGCCACCAGCAGCGTGGCCTACCTGAACTGGCACAACGGCCAGATCGAC |
| | | AACGAGCCCAAGCTGGACATGAACAAGCAGAAGATTAGCCCCGCCGAG |
| | | ATCCCCAACCCACTGCTGGGCCTGGAC |
| 172 | UbM_L7Ae_cb2 | ATGACCAGCCAGATTTTCGTCAAGACACTGACAGGCAAGACCATCACCC |
| | (2-95)_V5 (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCATGGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGCTGAAGTACAAGCCCCTGTTGAAGATCAGCAAGAACTG |
| | | CGAGGCCGCCATCCTGCGGGCCAGCAAGACCCGGCTGAACACCATCCGG |
| | | GCCTACGGCAGCACCGTGCCCAAGAGCAAGAGCTTCGAGCAGGACAGCC |
| | | GGAAGCGGACCCAGAGCTGGACCGCCCTGCGGGTGGGTGCAATCCTGGC |
| | | CGCCACCAGCAGCGTGGCCTACCTGAACTGGCACAACGGCCAGATCGAC |
| | | AACGAGCCCAAGCTGGACATGAACAAGCAGAAGATTAGCCCCGCCGAG |
| | | ATCCCCAACCCACTGCTGGGCCTGGAC |
| 173 | 3xUbVR_L7Ae_ | ATGACCAGCCAGATTTTCGTCAAGACACTGACAGGCAAGACCATCACCC |
| | V5 (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGCGAGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTACGGGCTAGCGCCAGTC |
| | | AGATCTTCGTGAAGACCCTCACCGGCAAGACGATTACCTTAGAGGTGGA |
| | | GCCTAGCGATACCATAGAGAACGTGAAGGCTAAGATCCAGGATAAGGA |
| | | GGGCATCCCTCCTGATCAACAACGGTTAATCTTTGCTGGCAAGCAGCTGG |
| | | AGGACGGACGGACCCTGAGCGATTACAATATCCAGAAGGAGAGCACCTT |
| | | GCACCTCGTGCTGCGGCTGAGAGGCGTCCGTGCCAGCGCCAGCTACGTG |
| | | CGGTTCGAGGTGCCCGAGGACATGCAGAACGAGGCCCTGAGCCTGCTGG |
| | | AGAAGGTGCGGGAGAGCGGCAAGGTGAAGAAGGGCACCAACGAGACCA |
| | | CCAAGGCCGTGGAGCGGGGTCTGGCTAAGCTGGTGTACATCGCCGAGGA |
| | | CGTGGACCCTCCCGAGATCGTGGCCCACCTGCCCCTGCTGTGCGAGGAG |
| | | AAGAACGTGCCCTACATCTACGTGAAGAGTAAGAACGACCTTGGCCGGG |
| | | CCGTGGGCATCGAGGTCCCCTGCGCCAGCGCTGCCATCATCAACGAGGG |
| | | CGAGCTGCGGAAGGAGCTGGGCAGCCTGGTGGAGAAGATCAAGGGCCT |
| | | GCAGAAGATCCCCAACCCACTGCTGGGCCTGGAC |
| 174 | 3xUbVV_L7Ae_ | ATGACCAGCCAGATTTTCGTCAAGACACTGACAGGCAAGACCATCACCC |
| | V5 (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGGTGGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTAGTGGCTAGCGCCAGTCA |
| | | GATCTTCGTGAAGACCCTCACCGGCAAGACGATTACCTTAGAGGTGGAG |
| | | CCTAGCGATACCATAGAGAACGTGAAGGCTAAGATCCAGGATAAGGAG |
| | | GGCATCCCTCCTGATCAACAACGGTTAATCTTTGCTGGCAAGCAGCTGGA |
| | | GGACGGACGGACCCTGAGCGATTACAATATCCAGAAGGAGAGCACCTTG |
| | | CACCTCGTGCTGCGGCTGAGAGGCGTCGTCGCCAGCGCCAGCTACGTGC |
| | | GGTTCGAGGTGCCCGAGGACATGCAGAACGAGGCCCTGAGCCTGCTGGA |
| | | GAAGGTGCGGGAGAGCGGCAAGGTGAAGAAGGGCACCAACGAGACCAC |
| | | CAAGGCCGTGGAGCGGGGTCTGGCTAAGCTGGTGTACATCGCCGAGGAC |
| | | GTGGACCCTCCCGAGATCGTGGCCCACCTGCCCCTGCTGTGCGAGGAGA |
| | | AGAACGTGCCCTACATCTACGTGAAGAGTAAGAACGACCTTGGCCGGGC |
| | | CGTGGGCATCGAGGTCCCCTGCGCCAGCGCTGCCATCATCAACGAGGGC |
| | | GAGCTGCGGAAGGAGCTGGGCAGCCTGGTGGAGAAGATCAAGGGCCTG |
| | | CAGAAGATCCCCAACCCACTGCTGGGCCTGGAC |
| 175 | UbR_L7Ae_V5 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCCGAGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGATCCCCAACCCACTGCTGGGCCTGGAC |
| 176 | UbK_L7Ae_V5 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCAAGGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGATCCCCAACCCACTGCTGGGCCTGGAC |
| 177 | PEST | ATGAGCCACGGCTTTCCTCCTGAGGTGGAGGAGCAGGACGACGGCACCC |
| | (mODC)_L7Ae_ | TGCCCATGAGCTGCGCCCAGGAGAGCGGCATGGACCGGCATCCCGCCGC |
| | V5 (nt) | CTGTGCCTCTGCCCGGATCAACGTGTACGTGCGGTTCGAGGTGCCCGAG |
| | | GACATGCAGAACGAGGCCCTGAGCCTGCTGGAGAAGGTGCGGGAGAGC |
| | | GGCAAGGTGAAGAAGGGCACCAACGAGACCACCAAGGCCGTGGAGCGG |
| | | GGTCTGGCTAAGCTGGTGTACATCGCCGAGGACGTGGACCCTCCCGAGA |
| | | TCGTGGCCCACCTGCCCCTGCTGTGCGAGGAGAAGAACGTGCCCTACAT |
| | | CTACGTGAAGAGTAAGAACGACCTTGGCCGGGCCGTGGGCATCGAGGTC |
| | | CCCTGCGCCAGCGCTGCCATCATCAACGAGGGCGAGCTGCGGAAGGAGC |
| | | TGGGCAGCCTGGTGGAGAAGATCAAGGGCCTGCAGAAGATCCCCAACCC |
| | | ACTGCTGGGCCTGGAC |
| 178 | UbD_L7Ae_V5 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCGACGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGATCCCCAACCCACTGCTGGGCCTGGAC |
| 179 | UbM_L7Ae_V5 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCATGGCCAGCTA |
| | | CGTGCGGTTCGAGGTGCCCGAGGACATGCAGAACGAGGCCCTGAGCCTG |
| | | CTGGAGAAGGTGCGGGAGAGCGGCAAGGTGAAGAAGGGCACCAACGAG |
| | | ACCACCAAGGCCGTGGAGCGGGGTCTGGCTAAGCTGGTGTACATCGCCG |
| | | AGGACGTGGACCCTCCCGAGATCGTGGCCCACCTGCCCCTGCTGTGCGA |
| | | GGAGAAGAACGTGCCCTACATCTACGTGAAGAGTAAGAACGACCTTGGC |
| | | CGGGCCGTGGGCATCGAGGTCCCCTGCGCCAGCGCTGCCATCATCAACG |
| | | AGGGCGAGCTGCGGAAGGAGCTGGGCAGCCTGGTGGAGAAGATCAAGG |
| | | GCCTGCAGAAGATCCCCAACCCACTGCTGGGCCTGGAC |
| 180 | 3xUbVR_Snu13 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGCGAGCCAGCGC |
| | | AAGCCAGATCTTTGTTAAGACCCTGACCGGGAAGACAATCACTCTGGAA |
| | | GTGGAGCCCAGCGACACTATCGAGAACGTTAAAGCTAAGATTCAAGACA |
| | | AGGAGGGGATTCCACCTGACCAGCAACGGCTGATTTTCGCCGGCAAACA |
| | | GCTGGAAGACGGCCGGACACTGAGCGACTACAACATTCAGAAAGAGTCC |
| | | ACCCTGCATCTGGTGCTTCGGCTGCGGGGTGTACGGGCTAGCGCCAGTC |
| | | AGATCTTCGTGAAGACCCTCACCGGCAAGACGATTACCTTAGAGGTGGA |
| | | GCCTAGCGATACCATAGAGAACGTGAAGGCTAAGATCCAGGATAAGGA |
| | | GGGCATCCCTCCTGATCAACAACGGTTAATCTTTGCTGGCAAGCAGCTGG |
| | | AGGACGGACGGACCCTGAGCGATTACAATATCCAGAAGGAGAGCACCTT |
| | | GCACCTCGTGCTGCGGCTGAGAGGCGTCCGTGCCAGCGCCAGCACCGAG |
| | | GCCGACGTGAACCCTAAGGCCTACCCTCTGGCCGACGCCCACCTGACCA |
| | | AGAAGCTGCTGGACCTGGTGCAGCAGAGCTGCAACTACAAGCAGCTGAG |
| | | AAAGGGCGCCAACGAGGCCACCAAGACCCTGAACAGAGGCATCAGCGA |
| | | GTTCATCGTGATGGCCGCCGACGCCGAGCCTCTGGAGATCATCCTGCACC |
| | | TGCCTCTGCTGTGCGAGGACAAGAACGTGCCTTACGTGTTCGTGAGAAG |
| | | CAAGCAGGCCCTGGGCAGAGCCTGCGGCGTGAGCAGACCTGTGATCGCC |
| | | TGCAGCGTGACCATCAAGGAGGGCAGCCAGCTGAAGCAGCAGATCCAG |
| | | AGCATCCAGCAGAGCATCGAGAGACTGCTGGTG |
| 181 | UbVR_Snu13 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGTGCGAGCCAGCGC |
| | | AAGCACCGAGGCCGACGTGAACCCTAAGGCCTACCCTCTGGCCGACGCC |
| | | CACCTGACCAAGAAGCTGCTGGACCTGGTGCAGCAGAGCTGCAACTACA |
| | | AGCAGCTGAGAAAGGGCGCCAACGAGGCCACCAAGACCCTGAACAGAG |
| | | GCATCAGCGAGTTCATCGTGATGGCCGCCGACGCCGAGCCTCTGGAGAT |
| | | CATCCTGCACCTGCCTCTGCTGTGCGAGGACAAGAACGTGCCTTACGTGT |
| | | TCGTGAGAAGCAAGCAGGCCCTGGGCAGAGCCTGCGGCGTGAGCAGAC |
| | | CTGTGATCGCCTGCAGCGTGACCATCAAGGAGGGCAGCCAGCTGAAGCA |
| | | GCAGATCCAGAGCATCCAGCAGAGCATCGAGAGACTGCTGGTG |
| 182 | UbR_Snu13 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCCGAGCCAGCAC |
| | | CGAGGCCGACGTGAACCCTAAGGCCTACCCTCTGGCCGACGCCCACCTG |
| | | ACCAAGAAGCTGCTGGACCTGGTGCAGCAGAGCTGCAACTACAAGCAGC |
| | | TGAGAAAGGGCGCCAACGAGGCCACCAAGACCCTGAACAGAGGCATCA |
| | | GCGAGTTCATCGTGATGGCCGCCGACGCCGAGCCTCTGGAGATCATCCT |
| | | GCACCTGCCTCTGCTGTGCGAGGACAAGAACGTGCCTTACGTGTTCGTGA |
| | | GAAGCAAGCAGGCCCTGGGCAGAGCCTGCGGCGTGAGCAGACCTGTGAT |
| | | CGCCTGCAGCGTGACCATCAAGGAGGGCAGCCAGCTGAAGCAGCAGATC |
| | | CAGAGCATCCAGCAGAGCATCGAGAGACTGCTGGTG |
| 183 | UbK_Snu13 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCAAGGCCAGCAC |
| | | CGAGGCCGACGTGAACCCTAAGGCCTACCCTCTGGCCGACGCCCACCTG |
| | | ACCAAGAAGCTGCTGGACCTGGTGCAGCAGAGCTGCAACTACAAGCAGC |
| | | TGAGAAAGGGCGCCAACGAGGCCACCAAGACCCTGAACAGAGGCATCA |
| | | GCGAGTTCATCGTGATGGCCGCCGACGCCGAGCCTCTGGAGATCATCCT |
| | | GCACCTGCCTCTGCTGTGCGAGGACAAGAACGTGCCTTACGTGTTCGTGA |
| | | GAAGCAAGCAGGCCCTGGGCAGAGCCTGCGGCGTGAGCAGACCTGTGAT |
| | | CGCCTGCAGCGTGACCATCAAGGAGGGCAGCCAGCTGAAGCAGCAGATC |
| | | CAGAGCATCCAGCAGAGCATCGAGAGACTGCTGGTG |
| 184 | UbM_Snu13 | ATGACCAGCCAGATTTTCGTCAAGACACTGACAGGCAAGACCATCACCC |
| | (nt) | TGGAGGTGGAACCCTCCGACACCATTGAGAACGTCAAGGCCAAGATACA |
| | | GGACAAGGAAGGCATACCTCCCGATCAGCAGAGGCTCATCTTCGCAGGC |
| | | AAGCAACTCGAGGACGGCAGGACCCTGTCTGACTATAACATCCAGAAAG |
| | | AAAGCACTCTGCACCTGGTTCTGCGGCTCCGCGGTGGCATGGCCAGCAC |
| | | CGAGGCCGACGTGAACCCTAAGGCCTACCCTCTGGCCGACGCCCACCTG |
| | | ACCAAGAAGCTGCTGGACCTGGTGCAGCAGAGCTGCAACTACAAGCAGC |
| | | TGAGAAAGGGCGCCAACGAGGCCACCAAGACCCTGAACAGAGGCATCA |
| | | GCGAGTTCATCGTGATGGCCGCCGACGCCGAGCCTCTGGAGATCATCCT |
| | | GCACCTGCCTCTGCTGTGCGAGGACAAGAACGTGCCTTACGTGTTCGTGA |
| | | GAAGCAAGCAGGCCCTGGGCAGAGCCTGCGGCGTGAGCAGACCTGTGAT |
| | | CGCCTGCAGCGTGACCATCAAGGAGGGCAGCCAGCTGAAGCAGCAGATC |
| | | CAGAGCATCCAGCAGAGCATCGAGAGACTGCTGGTG |
| 72 | Snu13 (nt) | ATGACCGAGGCCGACGTGAACCCTAAGGCCTACCCTCTGGCCGACGCCC |
| | | ACCTGACCAAGAAGCTGCTGGACCTGGTGCAGCAGAGCTGCAACTACAA |
| | | GCAGCTGAGAAAGGGCGCCAACGAGGCCACCAAGACCCTGAACAGAGG |
| | | CATCAGCGAGTTCATCGTGATGGCCGCCGACGCCGAGCCTCTGGAGATC |
| | | ATCCTGCACCTGCCTCTGCTGTGCGAGGACAAGAACGTGCCTTACGTGTT |
| | | CGTGAGAAGCAAGCAGGCCCTGGGCAGAGCCTGCGGCGTGAGCAGACC |
| | | TGTGATCGCCTGCAGCGTGACCATCAAGGAGGGCAGCCAGCTGAAGCAG |
| | | CAGATCCAGAGCATCCAGCAGAGCATCGAGAGACTGCTGGTG |
| 55 | L7Ae | MYVRFEVPEDMQNEALSLLEKVRESGKVKKGTNETTKAVERGLAKLVYIA |
| | | EDVDPPEIVAHLPLLCEEKNVPYIYVKSKNDLGRAVGIEVPCASAAIINEGEL |
| | | RKELGSLVEKIKGLQK |
| 185 | Timer_less | MYVRFEVPEDMQNEALSLLEKVRESGKVKKGTNETTKAVERGLAKLVYIA |
| | Timer_lesser | EDVDPPEIVAHLPLLCEEKNVPYIYVKSKNDLGRAVGIEVPCASAAIINEGEL |
| | Timer_least | RKELGSLVEKIKGLQKGKPIPNPLLGLDST |
| 186 | PEST | MSHGFPPEVEEQDDGTLPMSCAQESGMDRHPAACASARINV |
| 187 | UbR | MTSQIFVKTLTGKTITLEVEPSDTIENVKAKIQDKEGIPPDQQRLIFAGKQLED |
| | | GRTLSDYNIQKESTLHLVLRLRGGRAS |
| 188 | UbD | MTSQIFVKTLTGKTITLEVEPSDTIENVKAKIQDKEGIPPDQQRLIFAGKQLED |
| | | GRTLSDYNIQKESTLHLVLRLRGGDAS |
| 189 | ubK | MTSQIFVKTLTGKTITLEVEPSDTIENVKAKIQDKEGIPPDQQRLIFAGKQLED |
| | | GRTLSDYNIQKESTLHLVLRLRGGKAS |
| 190 | UbM | MTSQIFVKTLTGKTITLEVEPSDTIENVKAKIQDKEGIPPDQQRLIFAGKQLED |
| | | GRTLSDYNIQKESTLHLVLRLRGGMAS |
| 191 | cb2 | LKYKPLLKISKNCEAAILRASKTRLNTIRAYGSTVPKSKSFEQDSRKRTQSWT |
| | | ALRVGAILAATSSVAYLNWHNGQIDNEPKLDMNKQKISPAE |
| 192 | 3xUBVV | MTSQIFVKTLTGKTITLEVEPSDTIENVKAKIQDKEGIPPDQQRLIFAGKQLED |
| | | GRTLSDYNIQKESTLHLVLRLRGVVASASQIFVKTLTGKTITLEVEPSDTIEN |
| | | VKAKIQDKEGIPPDQQRLIFAGKQLEDGRTLSDYNIQKESTLHLVLRLRGVV |
| | | ASASQIFVKTLTGKTITLEVEPSDTIENVKAKIQDKEGIPPDQQRLIFAGKQLE |
| | | DGRTLSDYNIQKESTLHLVLRLRGVVASAS |
| 193 | 3xUbVr | MTSQIFVKTLTGKTITLEVEPSDTIENVKAKIQDKEGIPPDQQRLIFAGKQLED |
| | | GRTLSDYNIQKESTLHLVLRLRGVRASASQIFVKTLTGKTITLEVEPSDTIEN |
| | | VKAKIQDKEGIPPDQQRLIFAGKQLEDGRTLSDYNIQKESTLHLVLRLRGVR |
| | | ASASQIFVKTLTGKTITLEVEPSDTIENVKAKIQDKEGIPPDQQRLIFAGKQLE |
| | | DGRTLSDYNIQKESTLHLVLRLRGVRASAS |
| 194 | deGGFP | MVSKGEELFTGVVPILVELDGDVNGHKFSVSGEGEGDATYGKLTLKFICTT |
| | (ORF1) | GKLPVPWPTLVTTLTYGVQCFSRYPDHMKQHDFFKSAMPEGYVQERTIFFK |
| | deGGFP22 | DDGNYKTRAEVKFEGDTLVNRIELKGIDFKEDGNILGHKLEYNYNSHNVYI |
| | (ORF2) | MADKQKNGIKVNFKIRHNIEDGSVQLADHYQQNTPIGDGPVLLPDNHYLST |
| | | QSALSKDPNEKRDHMVLLEFVTAAGITLGMDELYKRSRDISHGFPPAVAAQ |
| | | DDGTLPMSCAQESGMDRHPAACASARINV |
| 195 | mCherry | MVSKGEEDNMAIIKEFMRFKVHMEGSVNGHEFEIEGEGEGRPYEGTQTAKL |
| | | KVTKGGPLPFAWDILSPQFMYGSKAYVKHPADIPDYLKLSFPEGFKWERVM |
| | | NFEDGGVVTVTQDSSLQDGEFIYKVKLRGTNFPSDGPVMQKKTMGWEASS |
| | | ERMYPEDGALKGEIKQRLKLKDGGHYDAEVKTTYKAKKPVQLPGAYNVNI |
| | | KLDITSHNEDYTIVEQYERAEGRHSTGGMDELYK |
| 196 | EPO | MGVHECPAWLWLLLSLLSLPLGLPVLGAPPRLICDSRVLERYLLEAKEAENI |
| | | TTGCAEHCSLNENITVPDTKVNFYAWKRMEVGQQAVEVWQGLALLSEAVL |
| | | RGQALLVNSSQPWEPLQLHVDKAVSGLRSLTTLLRALGAQKEAISPPDAAS |
| | | AAPLRTITADTFRKLFRVYSNFLRGKLKLYTGEACRTGDR |
| 197 | 3xUbVR_L7Ae_ | MTSQIFVKTLTGKTITLEVEPSDTIENVKAKIQDKEGIPPDQQRLIFAGKQLED |
| | cb2(2-95)_V5 | GRTLSDYNIQKESTLHLVLRLRGVRASASQIFVKTLTGKTITLEVEPSDTIEN |
| | | VKAKIQDKEGIPPDQQRLIFAGKQLEDGRTLSDYNIQKESTLHLVLRLRGVR |
| | | ASASQIFVKTLTGKTITLEVEPSDTIENVKAKIQDKEGIPPDQQRLIFAGKQLE |
| | | DGRTLSDYNIQKESTLHLVLRLRGVRASASYVRFEVPEDMQNEALSLLEKV |
| | | RESGKVKKGTNETTKAVERGLAKLVYIAEDVDPPEIVAHLPLLCEEKNVPYI |
| | | YVKSKNDLGRAVGIEVPCASAAIINEGELRKELGSLVEKIKGLQKLKYKPLL |
| | | KISKNCEAAILRASKTRLNTIRAYGSTVPKSKSFEQDSRKRTQSWTALRVGAI |
| | | LAATSSVAYLNWHNGQIDNEPKLDMNKQKISPAEIPNPLLGLD |
| 198 | 3xUbVV_L7Ae_ | MTSQIFVKTLTGKTITLEVEPSDTIENVKAKIQDKEGIPPDQQRLIFAGKQLED |
| | cb2(2-95)_V5 | GRTLSDYNIQKESTLHLVLRLRGVVASASQIFVKTLTGKTITLEVEPSDTIEN |
| | | VKAKIQDKEGIPPDQQRLIFAGKQLEDGRTLSDYNIQKESTLHLVLRLRGVV |
| | | ASASQIFVKTLTGKTITLEVEPSDTIENVKAKIQDKEGIPPDQQRLIFAGKQLE |
| | | DGRTLSDYNIQKESTLHLVLRLRGVVASASYVRFEVPEDMQNEALSLLEKV |
| | | RESGKVKKGTNETTKAVERGLAKLVYIAEDVDPPEIVAHLPLLCEEKNVPYI |
| | | YVKSKNDLGRAVGIEVPCASAAIINEGELRKELGSLVEKIKGLQKLKYKPLL |
| | | KISKNCEAAILRASKTRLNTIRAYGSTVPKSKSFEQDSRKRTQSWTALRVGAI |
| | | LAATSSVAYLNWHNGQIDNEPKLDMNKQKISPAEIPNPLLGLD |
| 199 | UbR_L7Ae_cb2 | MTSQIFVKTLTGKTITLEVEPSDTIENVKAKIQDKEGIPPDQQRLIFAGKQLED |
| | (2-95)_V5 | GRTLSDYNIQKESTLHLVLRLRGGRASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKLKYKPLLKIS |
| | | KNCEAAILRASKTRLNTIRAYGSTVPKSKSFEQDSRKRTQSWTALRVGAILA |
| | | ATSSVAYLNWHNGQIDNEPKLDMNKQKISPAEIPNPLLGLD |
| 200 | UbK_L7Ae_cb2 | MTSQIFVKTLTGKTITLEVEPSDTIENVKAKIQDKEGIPPDQQRLIFAGKQLED |
| | (2-95)_V5 | GRTLSDYNIQKESTLHLVLRLRGGKASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKLKYKPLLKIS |
| | | KNCEAAILRASKTRLNTIRAYGSTVPKSKSFEQDSRKRTQSWTALRVGAILA |
| | | ATSSVAYLNWHNGQIDNEPKLDMNKQKISPAEIPNPLLGLD |
| 201 | PEST | MSHGFPPEVEEQDDGTLPMSCAQESGMDRHPAACASARINVYVRFEVPED |
| | (mODC)_L7Ae_ | MQNEALSLLEKVRESGKVKKGTNETTKAVERGLAKLVYIAEDVDPPEIVAH |
| | cb2(2-95)_V5 | LPLLCEEKNVPYIYVKSKNDLGRAVGIEVPCASAAIINEGELRKELGSLVEKI |
| | | KGLQKLKYKPLLKISKNCEAAILRASKTRLNTIRAYGSTVPKSKSFEQDSRK |
| | | RTQSWTALRVGAILAATSSVAYLNWHNGQIDNEPKLDMNKQKISPAEIPNP |
| | | LLGLD |
| 202 | UbD_L7Ae_cb2 | MTSQIFVKTLTGKTITLEVEPSDTIENVKAKIQDKEGIPPDQQRLIFAGKQLED |
| | (2-95)_V5 | GRTLSDYNIQKESTLHLVLRLRGGDASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKLKYKPLLKIS |
| | | KNCEAAILRASKTRLNTIRAYGSTVPKSKSFEQDSRKRTQSWTALRVGAILA |
| | | ATSSVAYLNWHNGQIDNEPKLDMNKQKISPAEIPNPLLGLD |
| 203 | UbM_L7Ae_cb2 | MTSQIFVKTLTGKTITLEVEPSDTIENVKAKIQDKEGIPPDQQRLIFAGKQLED |
| | (2-95)_V5 | GRTLSDYNIQKESTLHLVLRLRGGMASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKLKYKPLLKIS |
| | | KNCEAAILRASKTRLNTIRAYGSTVPKSKSFEQDSRKRTQSWTALRVGAILA |
| | | ATSSVAYLNWHNGQIDNEPKLDMNKQKISPAEIPNPLLGLD |
| 204 | 3xUbVR_L7Ae_ | MTSQIFVKTLTGKTITLEVEPSDTIENVKAKIQDKEGIPPDQQRLIFAGKQLED |
| | V5 | GRTLSDYNIQKESTLHLVLRLRGVRASASQIFVKTLTGKTITLEVEPSDTIEN |
| | | VKAKIQDKEGIPPDQQRLIFAGKQLEDGRTLSDYNIQKESTLHLVLRLRGVR |
| | | ASASQIFVKTLTGKTITLEVEPSDTIENVKAKIQDKEGIPPDQQRLIFAGKQLE |
| | | DGRTLSDYNIQKESTLHLVLRLRGVRASASYVRFEVPEDMQNEALSLLEKV |
| | | RESGKVKKGTNETTKAVERGLAKLVYIAEDVDPPEIVAHLPLLCEEKNVPYI |
| | | YVKSKNDLGRAVGIEVPCASAAIINEGELRKELGSLVEKIKGLQKIPNPLLGL |
| | | D |
| 205 | 3xUbVV_L7Ae_ | MTSQIFVKTLTGKTITLEVEPSDTIENVKAKIQDKEGIPPDQQRLIFAGKQLED |
| | V5 | GRTLSDYNIQKESTLHLVLRLRGVVASASQIFVKTLTGKTITLEVEPSDTIEN |
| | | VKAKIQDKEGIPPDQQRLIFAGKQLEDGRTLSDYNIQKESTLHLVLRLRGVV |
| | | ASASQIFVKTLTGKTITLEVEPSDTIENVKAKIQDKEGIPPDQQRLIFAGKQLE |
| | | DGRTLSDYNIQKESTLHLVLRLRGVVASASYVRFEVPEDMQNEALSLLEKV |
| | | RESGKVKKGTNETTKAVERGLAKLVYIAEDVDPPEIVAHLPLLCEEKNVPYI |
| | | YVKSKNDLGRAVGIEVPCASAAIINEGELRKELGSLVEKIKGLQKIPNPLLGL |
| | | D |
| 206 | UbR_L7Ae_V5 | MTSQIFVKTLTGKTITLEVEPSDTIENVKAKIQDKEGIPPDQQRLIFAGKQLED |
| | | GRTLSDYNIQKESTLHLVLRLRGGRASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKIPNPLLGLD |
| 207 | UbK_L7Ae_V5 | MTSQIFVKTLTGKTITLEVEPSDTIENVKAKIQDKEGIPPDQQRLIFAGKQLED |
| | | GRTLSDYNIQKESTLHLVLRLRGGKASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKIPNPLLGLD |
| 208 | PEST | MSHGFPPEVEEQDDGTLPMSCAQESGMDRHPAACASARINVYVRFEVPED |
| | (mODC)_L7Ae_ | MQNEALSLLEKVRESGKVKKGTNETTKAVERGLAKLVYIAEDVDPPEIVAH |
| | V5 | LPLLCEEKNVPYIYVKSKNDLGRAVGIEVPCASAAIINEGELRKELGSLVEKI |
| | | KGLQKIPNPLLGLD |
| 209 | UbD_L7Ae_V5 | MTSQIFVKTLTGKTITLEVEPSDTIENVKAKIQDKEGIPPDQQRLIFAGKQLED |
| | | GRTLSDYNIQKESTLHLVLRLRGGDASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKIPNPLLGLD |
| 210 | UbM_L7Ae_V5 | MTSQIFVKTLTGKTITLEVEPSDTIENVKAKIQDKEGIPPDQQRLIFAGKQLED |
| | | GRTLSDYNIQKESTLHLVLRLRGGMASYVRFEVPEDMQNEALSLLEKVRES |
| | | GKVKKGTNETTKAVERGLAKLVYIAEDVDPPEIVAHLPLLCEEKNVPYIYV |
| | | KSKNDLGRAVGIEVPCASAAIINEGELRKELGSLVEKIKGLQKIPNPLLGLD |
| 211 | 3xUbVR_Snu13 | MTSQIFVKTLTGKTITLEVEPSDTIENVKAKIQDKEGIPPDQQRLIFAGKQLED |
| | | GRTLSDYNIQKESTLHLVLRLRGVRASASQIFVKTLTGKTITLEVEPSDTIEN |
| | | VKAKIQDKEGIPPDQQRLIFAGKQLEDGRTLSDYNIQKESTLHLVLRLRGVR |
| | | ASASQIFVKTLTGKTITLEVEPSDTIENVKAKIQDKEGIPPDQQRLIFAGKQLE |
| | | DGRTLSDYNIQKESTLHLVLRLRGVRASASTEADVNPKAYPLADAHLTKKL |
| | | LDLVQQSCNYKQLRKGANEATKTLNRGISEFIVMAADAEPLEIILHLPLLCE |
| | | DKNVPYVFVRSKQALGRACGVSRPVIACSVTIKEGSQLKQQIQSIQQSIERLL |
| | | V |
| 212 | UbVR_Snu13 | MTSQIFVKTLTGKTITLEVEPSDTIENVKAKIQDKEGIPPDQQRLIFAGKQLED |
| | | GRTLSDYNIQKESTLHLVLRLRGVRASASTEADVNPKAYPLADAHLTKKLL |
| | | DLVQQSCNYKQLRKGANEATKTLNRGISEFIVMAADAEPLEIILHLPLLCED |
| | | KNVPYVFVRSKQALGRACGVSRPVIACSVTIKEGSQLKQQIQSIQQSIERLLV |
| 213 | UbR_Snu13 | MTSQIFVKTLTGKTITLEVEPSDTIENVKAKIQDKEGIPPDQQRLIFAGKQLED |
| | | GRTLSDYNIQKESTLHLVLRLRGGRASTEADVNPKAYPLADAHLTKKLLDL |
| | | VQQSCNYKQLRKGANEATKTLNRGISEFIVMAADAEPLEIILHLPLLCEDKN |
| | | VPYVFVRSKQALGRACGVSRPVIACSVTIKEGSQLKQQIQSIQQSIERLLV |
| 214 | UbK_Snu13 | MTSQIFVKTLTGKTITLEVEPSDTIENVKAKIQDKEGIPPDQQRLIFAGKQLED |
| | | GRTLSDYNIQKESTLHLVLRLRGGKASTEADVNPKAYPLADAHLTKKLLDL |
| | | VQQSCNYKQLRKGANEATKTLNRGISEFIVMAADAEPLEIILHLPLLCEDKN |
| | | VPYVFVRSKQALGRACGVSRPVIACSVTIKEGSQLKQQIQSIQQSIERLLV |
| 215 | UbM_Snu13 | MTSQIFVKTLTGKTITLEVEPSDTIENVKAKIQDKEGIPPDQQRLIFAGKQLED |
| | | GRTLSDYNIQKESTLHLVLRLRGGMASTEADVNPKAYPLADAHLTKKLLDL |
| | | VQQSCNYKQLRKGANEATKTLNRGISEFIVMAADAEPLEIILHLPLLCEDKN |
| | | VPYVFVRSKQALGRACGVSRPVIACSVTIKEGSQLKQQIQSIQQSIERLLV |
| 71 | Snu13 | MTEADVNPKAYPLADAHLTKKLLDLVQQSCNYKQLRKGANEATKTLNRGI |
| | | SEFIVMAADAEPLEIILHLPLLCEDKNVPYVFVRSKQALGRACGVSRPVIACS |
| | | VTIKEGSQLKQQIQSIQQSIERLLV |

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A composition comprising:
(a) a first polynucleotide comprising (i) a repressor binding element, and (ii) an open reading frame encoding a first polypeptide; and
(b) a second polynucleotide comprising a sequence encoding a fusion polypeptide comprising (i) a repressor that binds to the repressor binding element, and (ii) a destabilization domain, wherein binding of the repressor to the repressor binding element reduces translation of the first polypeptide from the first polynucleotide, and wherein the destabilization domain enhances degradation of the repressor.

2. The composition of claim 1, wherein the destabilization domain comprises a ubiquitin tag.

3. The composition of claim 1 or 2, wherein the first polynucleotide is an mRNA and comprises a polyA tail.

4. The composition of claim 1 or 2, wherein the first polynucleotide is a DNA.

5. The composition of any one of claims 1 to 4, wherein the second polynucleotide is an mRNA and comprises a polyA tail.

6. The composition of any one of claims 1 to 4, wherein the second polynucleotide is a DNA.

7. The composition of any one of claims 1 to 4, wherein the second polynucleotide is a circular RNA.

8. The composition of claim 1 or 2, wherein the first and second polynucleotides are DNA and are encoded in a single plasmid.

9. The composition of any one of claims 1-8, wherein the repressor binding element comprises a kink-turn forming sequence.

10. The composition of claim 9, wherein the repressor binding element is selected from the group consisting of PRE, PRE2, MS2, PP7, BoxB, U1A hairpin, and 7SK.

11. The composition of any one of claims 1-10, wherein the repressor is the 50S ribosomal L7Ae protein, the 15.5 kd repressor, Pumilio and FBF (PUF) protein, PUF2 protein, MBP-LacZ, MBP, PCP, Lambda N, U1A, LARP7, Snu13, or variants thereof.

12. The composition of any one of claims 2 to 11, wherein the ubiquitin tag is 3XUbVR, 3XUbVV, UbR, UbK, PEST, UbD, or UbM.

13. The composition of claim 12, wherein the fusion polypeptide further comprises a cytochrome b2 region.

14. The composition of any one of claims 1-13, wherein the first polypeptide is a secreted protein, a membrane-bound protein, or an intercellular protein.

15. The composition of any one of claims 1-14, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

16. The composition of any one of claims 1-15, wherein (a) and (b) are in separate dosage forms packaged together.

17. The composition of any one of claims 1-15, wherein (a) and (b) are in a unit dosage form.

18. A method of expressing a first polypeptide in a cell, the method comprising contacting the cell with the composition of any one of claims 1-17.

19. A method of expressing a first polypeptide in a cell, the method comprising contacting the cell with:
(a) a first polynucleotide comprising (i) a repressor binding element, and (ii) an open reading frame encoding a first polypeptide; and
(b) a second polynucleotide comprising a sequence encoding a fusion polypeptide comprising (i) a repressor that binds to the repressor binding element, and (ii) a destabilization domain,
wherein binding of the repressor to the repressor binding element reduces translation of the first polypeptide from the first polynucleotide, and wherein the destabilization domain enhances degradation of the repressor.

20. A method of expressing a first polypeptide in a subject, the method comprising administering to the subject:
(a) a first polynucleotide comprising (i) a repressor binding element, and (ii) an open reading frame encoding a first polypeptide; and
(b) a second polynucleotide comprising a sequence encoding a fusion polypeptide comprising (i) a repressor that binds to the repressor binding element, and (ii) a destabilization domain,
wherein binding of the repressor to the repressor binding element reduces translation of the first polypeptide from the first polynucleotide, and wherein the destabilization domain enhances degradation of the repressor.

21. The method of any one of claims 18-20, wherein the cell is a liver cell, a muscle cell, immune cell or a neuron.

22. The method of any one of claims 18-21, wherein the first polynucleotide is a DNA or an mRNA and the second polynucleotide is a DNA or an mRNA.

23. A composition comprising
(a) a first polynucleotide comprising (i) a repressor binding element, and (ii) an open reading frame encoding a first polypeptide;
(b) a second polynucleotide comprising an open reading frame encoding a second polypeptide; and
(c) a third polynucleotide comprising a sequence encoding a fusion polypeptide comprising (i) a repressor that binds to the repressor binding element, and (ii) a destabilization domain; and
wherein binding of the repressor to the repressor binding element reduces translation of the first polypeptide from the first polynucleotide, and wherein the destabilization domain enhances degradation of the repressor.

24. A composition comprising
(a) a first polynucleotide comprising (i) a first repressor binding element, and (ii) an open reading frame encoding a first polypeptide;
(b) a second polynucleotide comprising (i) a second repressor binding element, and (ii) an open reading frame encoding a second polypeptide;
(c) a third polynucleotide comprising a sequence encoding a first fusion polypeptide comprising (i) a first repressor that binds to the first repressor binding element, and (ii) a first destabilization domain; and
(d) a fourth polynucleotide comprising a sequence encoding a second fusion polypeptide comprising (i) a second repressor that binds to the second repressor binding element, and (ii) a second destabilization domain; and
wherein binding of the first and second repressors to the first and second repressor binding elements respectively reduce translation of the first and second polypeptides from the first and second polynucleotides, and wherein the first and second destabilization domains respectively degrade the first and second repressors at different rates.

25. A composition comprising
(a) a first polynucleotide comprising (i) a first repressor binding element, and (ii) an open reading frame encoding a first polypeptide;
(b) a second polynucleotide comprising (i) a second repressor binding element, and (ii) an open reading frame encoding a second polypeptide;
(c) a third polynucleotide comprising an open reading frame encoding a third polypeptide;
(d) a fourth polynucleotide comprising a sequence encoding a first fusion polypeptide comprising (i) a first repressor that binds to the first repressor binding element, and (ii) a first destabilization domain; and
(e) a fifth polynucleotide comprising a sequence encoding a second fusion polypeptide comprising (i) a second repressor that binds to the second repressor binding element, and (ii) a second destabilization domain; and
wherein binding of the first and second repressors to the first and second repressor binding elements respectively reduce translation of the first and second polypeptides from the first and second polynucleotides, and wherein the first and second destabilization domains respectively degrade the first and second repressors at different rates.
